(19) United States
(12) Patent Application Publication
(10) Pub. No.: US 2026/0297669 A1
(43) Pub. Date: Oct. 1, 2026

(54) METHODS FOR ANALYZING DNA MOLECULES IN SINGLE CELLS

(71) Applicant: Cellanome, Inc., Foster City, CA (US)

(72) Inventors: Mostafa RONAGHI, Atherton, CA (US); Tarun Kumar KHURANA, Fremont, CA (US); Craig BETTS, San Jose, CA (US)

(21) Appl. No.: 19/633,672

(22) Filed: Mar. 30, 2026

Related U.S. Application Data
(60) Provisional application No. 63/781,203, filed on Mar. 31, 2025.

(51) Int. Cl. C12Q 1/6874; C12Q 1/6841

(52) U.S. Cl. CPC C12Q 1/6874; C12Q 1/6841

(57) ABSTRACT

Provided herein are methods and systems for determining the copy number and locations of nucleic acid sequences within DNA molecules. The methods can utilize nucleic acid capture probes to capture fragments of DNA molecules or nucleic acids that include complements of sequences from the DNA molecule. The nucleic acid capture probes can be extended using the fragments or the nucleic acids as templates. The nucleic acid capture probes can include barcode sequences that can be associated with specific cells or groups of cells.

### CROSS REFERENCE

This application claims the benefit of U.S. Provisional Application No. 63/781,203 filed Mar. 31, 2025, which is incorporated herein by reference in its entirety.

### BACKGROUND

Copy number variants, nucleic acid sequences that vary in integer copy number between genomes, are ubiquitous across domains of life. The integer copy number of genes, alleles, and certain noncoding sequences can acutely impact cell phenotype and behavior. Even within clonal populations, copy number variation can also differ greatly among cells. Accordingly, methods that measure population-level copy number variation can be incapable of identifying heterogeneity within subpopulations or individual cells. There is thus a need for methods that accurately quantitate nucleic acid sequence copy number in individual cells.

### SUMMARY

In one embodiment, the present disclosure provides a method for DNA analysis, the method comprising: (a) hybridizing a first restriction enzyme to a first restriction enzyme binding site of a DNA molecule of a cell; (b) hybridizing a second restriction enzyme to a second restriction enzyme binding site of the DNA molecule, wherein a first handle or a portion of the first handle and a target nucleic acid sequence are disposed between the first restriction enzyme binding site and the second restriction enzyme binding site of the DNA molecule; (c) cleaving the DNA molecule with the first and second restriction enzymes, wherein the cleaving generates a nucleic acid fragment comprising: i) the first handle or a portion of the first handle, ii) the target nucleic acid sequence, and iii) a flanking DNA sequence from the DNA molecule disposed at an end of the nucleic acid fragment; (d) hybridizing the nucleic acid fragment or an amplicon generated from the nucleic acid fragment to a nucleic acid capture probe; (e) extending the nucleic acid capture probe using at least a portion of the nucleic acid fragment or the amplicon as a template, thereby generating an extended nucleic acid capture probe comprising a complement of the target nucleic acid sequence and a complement of the flanking DNA sequence; and (f) sequencing the extended nucleic acid capture probe or a nucleic acid generated from the extended nucleic acid capture probe.

In one aspect, the method further comprises inputting the cell into the fluidic device. In another aspect, the nucleic acid fragment further comprises an additional flanking DNA sequence from the DNA molecule, wherein the flanking DNA sequence and the additional flanking DNA sequence are disposed at opposite ends of the nucleic acid fragment. In a further aspect, the first restriction enzyme binding site is disposed within the first handle. In a particular aspect, the first restriction enzyme binding site is disposed at a first end of the first handle, the target nucleic acid sequence is coupled to a second end of the first handle, and the first end of the first handle is opposite the second end of the first handle.

In some aspects, the nucleic acid fragment further comprises a second handle or a portion of the second handle, and wherein the nucleic acid fragment comprises, in order: i) the flanking DNA sequence, the first handle, the target nucleic acid sequence, and the portion of the second handle; ii) the flanking DNA sequence, the first handle, the target nucleic acid sequence, and the second handle; iii) the first handle, the target nucleic acid sequence, the second handle, and the flanking DNA sequence; iv) the portion of the first handle, the target nucleic acid sequence, the second handle, and the flanking DNA sequence; or v) the flanking DNA sequence, the first handle, the target nucleic acid sequence, the second handle, and an additional flanking DNA sequence from the DNA molecule. In certain aspects, the second handle comprises a reverse complement of the first handle.

In an additional aspect, the DNA molecule is inside of the cell during the cleaving, and the method further comprises releasing the nucleic acid fragment from the cell following the cleaving. In a further aspect, the method further comprises releasing the DNA molecule from the cell prior to the cleaving. In a certain aspect, the releasing comprises lysis. In another aspect, the method further comprises inputting the cell into the fluidic device and synthesizing a chamber that co-encloses the cell with the nucleic acid capture probe.

In particular aspects, the DNA molecule comprises genomic DNA, plasmid DNA, vector DNA, extrachromosomal circular DNA (eccDNA), or a combination thereof. In some aspects, the target nucleic acid sequence comprises a knock-in gene. In certain aspects, the cell comprises a plurality of instances of the target nucleic acid sequence.

In a further aspect, the first handle, the portion of the first handle, a second handle disposed adjacent to the target nucleic acid sequence and comprising the second restriction enzyme binding site, or a portion of the second handle of the nucleic acid fragment hybridizes to the nucleic acid capture probe. In another aspect, the extended nucleic acid capture probe comprises a complement of the first handle, the portion of the first handle, a second handle disposed adjacent to the target nucleic acid sequence and comprising the second restriction enzyme binding site, a portion of the second handle, or a combination thereof. In a particular aspect, the nucleic acid fragment further comprises a unique molecular identifier. In one aspect, the nucleic acid capture probe comprises a spatial barcode associated with a location of the nucleic acid capture probe within the fluidic device

In some aspects, the method further comprises hybridizing the extended capture probe to a barcode primer, extending the extended capture probe using at least a portion of the barcode primer as a template, and extending the barcode primer using at least a portion of the extended capture probe as a template. In one such aspect, the barcode primer comprises a spatial barcode, wherein the extended capture probe is extended using at least a portion of the spatial barcode as a template. In certain aspects, the method further comprises amplifying the nucleic acid fragment, thereby generating the amplicon. In one such aspect, the method further comprises circularizing the nucleic acid fragment prior to the amplifying, and wherein the amplifying comprises amplifying the circularized nucleic acid fragment. In some aspects, the amplicon comprises circular DNA. In particular aspects, the method further comprises linearizing the amplicon prior to the hybridizing of the amplicon to the nucleic acid capture probe in (d). In additional aspects, the linearizing comprises cleaving the amplicon. In further aspects, the amplicon comprises linear DNA. In a certain aspect, the amplicon comprises multiple complements of the nucleic acid fragment. In another aspect, the method further comprises cleaving the amplicon into amplicon fragments, wherein each amplicon fragment contains a single complement of the nucleic acid fragment. In a further aspect, the first restriction enzyme, the second restriction enzyme, or the first and second restriction enzymes are type II restriction enzymes.

In further aspects, the method further comprises determining a copy number of the target nucleic acid sequence of the DNA molecule. In certain aspects, the determining the copy number of the target nucleic acid sequence comprises normalizing sequencing counts using the flanking DNA sequence. In additional aspects, the nucleic acid fragment further comprises a unique molecular identifier sequence, and the determining the copy number of the target nucleic acid sequence comprises normalizing sequencing counts using the unique molecular identifier sequence. In some aspects, the method further comprises determining a location of the target nucleic acid sequence of the DNA molecule using the flanking DNA sequence. In additional aspects, the extending in (e) comprises a DNA polymerase. In further aspects, the first restriction enzyme is identical to the second restriction enzyme, and wherein the first restriction enzyme binding site is identical to the second restriction enzyme binding site. In some aspects, the first restriction enzyme is different than the second restriction enzyme, and wherein the first restriction enzyme binding site is different than the second restriction enzyme binding site.

In some aspects, the nucleic acid capture probe is coupled to a surface of the fluidic device. In another aspect, the method further comprises cleaving the nucleic acid capture probe or the extended nucleic acid capture probe from the surface of the fluidic device.

In another embodiment, the present disclosure provides a method for DNA analysis, the method comprising: hybridizing a circularization probe to a DNA molecule of a cell, wherein: i) a first primer sequence of the circularization probe hybridizes to a first handle on a first strand of the DNA molecule, ii) a second primer sequence of the circularization probe hybridizes to a second handle on the first strand of the DNA molecule, and iii) a target nucleic acid sequence is disposed between the first handle and the second handle within the first strand of the DNA molecule; extending the first primer sequence using at least a portion of the target nucleic acid sequence and the at least one unique molecular identifier sequence as a template, thereby generating a 3′ extension coupled to the first primer sequence; ligating the 3′ extension to the second primer sequence, thereby circularizing the circularization probe; amplifying the circularized circularization probe, thereby generating amplicons of the circularization probe; hybridizing the amplicons to nucleic acid capture probes; extending the nucleic acid capture probes using at least a portion of the amplicons as templates, thereby generating an extended nucleic acid capture probe comprising a complement of the target nucleic acid sequence; and sequencing the extended nucleic acid capture probes or nucleic acids generated from the extended nucleic acid capture probes.

In a first aspect, a unique molecular identifier sequence is disposed between the first handle and the second handle within the first strand of the DNA molecule. In another aspect, the circularization probe comprises a unique molecular identifier. In an additional aspect, the method further comprises inputting the cell into the fluidic device. In a further aspect, the amplicons are linear amplicons. In certain aspects, the amplicons are circular amplicons, and wherein the method further comprises cleaving the circular amplicons, thereby generating linear amplicons. In some aspects, the circularization probe further comprises: i) an additional unique molecular identifier sequence; ii) a restriction enzyme binding site, wherein the cleaving comprises binding a nuclease to the restriction enzyme binding site; iii) a capture sequence, wherein the hybridizing the linear amplicons to nucleic acid capture probes comprises hybridizing the capture sequence or a complement of the capture sequence to the nucleic acid capture probes; iv) one or more barcode sequences; or v) a combination thereof.

In some aspects, the nucleic acid capture probe is coupled to a surface of the fluidic device. In another aspect, the method further comprises cleaving the nucleic acid capture probe or the extended nucleic acid capture probe from the surface of the fluidic device.

An additional embodiment of the present disclosure provides a method for DNA analysis, the method comprising: hybridizing a primer to a sequence of interest of a DNA molecule of a cell, wherein the sequence of interest comprises a target nucleic acid sequence, and wherein the primer comprises a first handle, wherein the DNA molecule comprises a flanking DNA sequence, and wherein the flanking DNA sequence is adjacent to the sequence of interest; extending the primer using: i) the target nucleic acid sequence or a portion of the target nucleic acid sequence as a template, ii) the flanking DNA sequence or a portion of the flanking DNA sequence as a template, or iii) a combination thereof, thereby generating an extended primer; hybridizing a random primer to the extended primer; extending the random primer using at least a portion of the extended primer as a template, thereby generating an extended random primer comprising a complement of the handle; hybridizing the complement of the handle of the extended random primer to a nucleic acid capture probe; extending the nucleic acid capture probe using at least a portion of the extended random primer as a template, thereby generating an extended nucleic acid capture probe; and sequencing the extended nucleic acid capture probe or a nucleic acid generated from the extended nucleic acid capture probe.

In certain aspects, the method further comprises inputting the cell into the fluidic device. In further aspects, the DNA molecule comprises genomic DNA, plasmid DNA, vector DNA, extrachromosomal circular DNA (eccDNA), or a combination thereof. In some aspects, the target nucleic acid sequence comprises a knock-in gene. In additional aspects, the DNA molecule comprises a plurality of instances of the target nucleic acid sequence. In some aspects, the primer hybridizes to the target nucleic acid sequence. In further aspects, the sequence of interest comprises a second handle, wherein the second handle is positioned between the target nucleic acid sequence and the flanking DNA sequence, wherein the primer hybridizes to the second handle.

In a particular aspect, the method further comprises determining a copy number of the sequence of interest of the DNA molecule. In one such aspect, the determining the copy number of the target nucleic acid sequence comprises normalizing sequencing counts using the flanking DNA sequence or sequence counts of a complement of the flanking DNA sequence. In another aspect, the sequence of interest further comprises a unique molecular identifier sequence, wherein the extending comprises extending the primer using the unique molecular identifier sequence as a template, and wherein the determining the copy number of the target nucleic acid sequence comprises normalizing sequencing counts using the unique molecular identifier sequence. In a further aspect, the method further comprises determining a location of the sequence of interest of the DNA molecule using the flanking DNA sequence or a complement of the flanking DNA sequence.

In some aspects, the extending comprises using a DNA polymerase. In additional aspects, the nucleic acid capture probe comprises a spatial barcode associated with the nucleic acid capture probe. In certain aspects, method further comprises hybridizing the extended capture probe to a barcode primer, extending the extended capture probe using at least a portion of the barcode primer as a template, and extending the barcode primer using at least a portion of the extended capture probe as a template. In particular aspects, the barcode primer comprises a spatial barcode.

In some aspects, the nucleic acid capture probe is coupled to a surface of the fluidic device. In another aspect, the method further comprises cleaving the nucleic acid capture probe or the extended nucleic acid capture probe from the surface of the fluidic device.

A further embodiment of the present disclosure provides a method for DNA analysis, the method comprising: hybridizing a primer to a sequence of interest in a DNA molecule of a cell, wherein the sequence of interest comprises a handle and a target nucleic acid sequence, wherein the primer hybridizes to the handle or at a position that is 3′ relative to the handle within the sequence of interest, wherein the DNA molecule comprises a flanking DNA sequence, and wherein the flanking DNA sequence is adjacent to the sequence of interest; extending the primer using the flanking DNA sequence as a template, thereby generating an extended primer comprising a complement of the handle and a complement of the flanking DNA sequence; hybridizing a random primer to the extended primer; extending the random primer using at least a portion of the extended primer, thereby generating an extended random primer comprising the handle and the flanking DNA sequence; hybridizing the handle of the extended random primer to a nucleic acid capture probe; extending the nucleic acid capture probe using at least a portion of the extended random primer as a template, thereby generating an extended nucleic acid capture probe comprising a complement of the flanking DNA sequence; and sequencing the extended nucleic acid capture probe or a nucleic acid generated from the extended nucleic acid capture probe.

In one aspect, the method further comprises inputting the cell into the fluidic device, wherein the cell comprises the DNA molecule. In another aspect, the target nucleic acid sequence comprises a gene. In an additional aspect, the target nucleic acid sequence is 3′ relative to the handle within the sequence of interest. In a further aspect, the handle comprises a poly A sequence, and wherein the handle of the extended random primer hybridizes to a polyT sequence of the nucleic acid capture probe.

In some aspects, the nucleic acid capture probe is coupled to a surface of the fluidic device. In another aspect, the method further comprises cleaving the nucleic acid capture probe or the extended nucleic acid capture probe from the surface of the fluidic device.

In an additional embodiment, the present disclosure provides a fluidic device, comprising: one or more nucleic acid capture probes disposed on a planar surface of the fluidic device, wherein the one or more nucleic acid capture probes comprise one or more capture sequences configured to hybridize to one or more nucleic acid fragments from a DNA molecule from one or more cells or amplicons of the one or more nucleic acid fragments, wherein the one or more nucleic acid fragments comprise: i) a handle or a portion of the handle, ii) a target nucleic acid sequence, and iii) a flanking DNA sequence from the DNA molecule disposed at an end of the nucleic acid fragment.

In some aspects, the one or more nucleic acid fragments further comprise an additional flanking DNA sequence from the DNA molecule, wherein the flanking DNA sequence and the additional flanking DNA sequence are disposed at opposite ends of the nucleic acid fragment. In further aspects, the one or more nucleic acid fragments further comprise an additional handle or a portion of the additional handle, and wherein the nucleic acid fragment comprises, in order: i) the flanking DNA sequence, the handle, the target nucleic acid sequence, and the portion of the additional handle; ii) the flanking DNA sequence, the handle, the target nucleic acid sequence, and the additional handle; iii) the handle, the target nucleic acid sequence, the additional handle, and the flanking DNA sequence; iv) the portion of the handle, the target nucleic acid sequence, the additional handle, and the flanking DNA sequence; or v) the flanking DNA sequence, the handle, the target nucleic acid sequence, the additional handle, and an additional flanking DNA sequence from the DNA molecule.

In an additional aspect, the additional handle comprises a reverse complement of the handle. In another aspect, the fluidic device comprises (i) a first chamber co-enclosing a first nucleic acid capture probe of the one or more nucleic acid capture probes and a first nucleic acid fragment of the one or more nucleic acid fragments and (ii) a second chamber co-enclosing a second nucleic acid capture probe of the one or more nucleic acid capture probes and a second nucleic acid fragment of the one or more nucleic acid fragments, wherein the first nucleic acid fragment is from a first cell of the one or more cells, and wherein the second nucleic acid fragment is from a second cell of the one or more cells. In a further aspect, the first nucleic acid capture probes comprises a first spatial barcode associated with a location of the first nucleic acid capture probe within the fluidic device, and wherein the second nucleic acid capture probe comprises a second spatial barcode associated with a location of the second nucleic acid capture probe within the fluidic device, and wherein the first spatial barcode and the second spatial barcode are different. In a particular aspect, the DNA molecule comprises genomic DNA, plasmid DNA, vector DNA, extrachromosomal circular DNA (eccDNA), or a combination thereof. In another aspect, the target nucleic acid sequence comprises a knock-in gene. In a certain aspect, a cell of the one or more cells comprises a plurality of instances of the target nucleic acid sequence.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic illustration of a portion of a channel disposed in a fluidic device, according to some embodiments.

FIG. 2A is an illustration of a portion of a system as provided herein including an energy source, according to some embodiments.

FIG. 2B is an illustration of a polymer matrix being formed around a biological component in a portion of a system as provided herein, according to some embodiments.

FIG. 2C is an illustration of a method of forming a polymer matrix around a biological component in a portion of the system as provided herein, according to some embodiments.

FIG. 3A is an illustration of a top view of the bottom layer of a flow cell.

FIG. 3B is an illustration of a top view of the spacer layer with a cut-out region suitable for use as part of the flow cell.

FIG. 3C is an illustration of a top view of the top layer of the flow cell where the top layer has an inlet and outlet opening.

FIG. 3D is an illustration of a cross-sectional side view of a top layer and a bottom layer sandwiching a spacer layer to form multiple channels of a flow cell.

FIG. 4A is an illustration of a system with a flow cell and an imaging apparatus that are capable of implementing methods of the present disclosure.

FIG. 4B is an illustration of a flow cell, including a blown-up view of a portion of a channel containing cells disposed in hydrogel chambers.

FIG. 5 is a schematic of a computer system that is configured to perform methods described herein.

FIG. 6A depicts a DNA molecule that includes multiple instances of a target nucleic acid.

FIG. 6B depicts the DNA molecule of FIG. 6A with restriction enzymes coupled to each instance of the target nucleic acid.

FIG. 6C depicts nucleic acid fragments cleaved from the DNA molecule of FIG. 6A, wherein each fragment includes a copy of the target nucleic acid.

FIG. 6D illustrates a circularized copy of the nucleic acid fragment from FIG. 6C.

FIG. 6E illustrates the nucleic acid fragment of FIG. 6C or an amplicon generated from the nucleic acid fragment coupled to a nucleic acid capture probe.

FIG. 6F illustrates the nucleic acid fragment (or the amplicon generated from the nucleic acid fragment) and nucleic acid capture probe from FIG. 6E following extension of the nucleic acid fragment using the nucleic acid capture probe as a template and extension of the nucleic acid capture probe using the nucleic acid fragment as a template.

FIG. 6G illustrates the extended nucleic acid fragment and extended nucleic acid capture probe from FIG. 6F following further extension of the extended nucleic acid capture probe using a template-switch oligonucleotide as a template.

FIG. 6H illustrates the template-switch oligonucleotide following extension using the further extended nucleic acid capture probe from FIG. 6G as a template.

FIG. 7A depicts a DNA molecule that includes multiple instances of a target nucleic acid.

FIG. 7B depicts the DNA molecule of FIG. 7A with first and second restriction enzymes coupled to first and second sequences of the target nucleic acids.

FIG. 7C depicts nucleic acid fragments cleaved from the DNA molecule of FIG. 7A, wherein each fragment includes a copy of the target nucleic acid.

FIG. 7D illustrates a circularized copy of the nucleic acid fragment from FIG. 7C.

FIG. 7E illustrates the nucleic acid fragment of FIG. 7C or an amplicon generated from the nucleic acid fragment coupled to a nucleic acid capture probe.

FIG. 7F illustrates the nucleic acid fragment (or the amplicon generated from the nucleic acid fragment) and nucleic acid capture probe from FIG. 7E following extension of the nucleic acid fragment using the nucleic acid capture probe as a template and extension of the nucleic acid capture probe using the nucleic acid fragment as a template.

FIG. 7G illustrates the extended nucleic acid fragment and extended nucleic acid capture probe from FIG. 7F following further extension of the extended nucleic acid capture probe using a template-switch oligonucleotide as a template.

FIG. 7H illustrates the template-switch oligonucleotide following extension using the further extended nucleic acid capture probe from FIG. 7G as a template.

FIG. 8A depicts a cell and nucleic acid capture probes co-enclosed in a chamber within a fluidic device.

FIG. 8B depicts the fluidic device of FIG. 8A following lysis of the cell, as well as blown up views of genomic DNA released from the cell and an instance of a target nucleic acid within the genomic DNA.

FIG. 8C depicts primer extension using an instance of the target nucleic acid of FIG. 8B as a template followed by random primer extension in which random primers are extended using the extended primers as templates.

FIG. 8D depicts an extended random primer from FIG. 8C coupled to a nucleic acid capture probe.

FIG. 8E depicts the extended random primer and nucleic acid capture probe of FIG. 8D following extension of the nucleic acid capture probe using the extended random primer as a template and further extension of the extended random primer using the nucleic acid capture probe as a template.

FIG. 9A depicts a cell and nucleic acid capture probes co-enclosed in a chamber within a fluidic device.

FIG. 9B depicts the fluidic device of FIG. 8A following lysis of the cell, as well as blown up views of genomic DNA released from the cell and an instance of a target nucleic acid within the genomic DNA, illustrating the handles, unique molecular identifiers, and sequence of interest within the target nucleic acid.

FIG. 9C depicts primer extension using handles and unique molecular identifiers of the target nucleic acid of FIG. 9B as a template followed by random primer extension using the extended primers as templates.

FIG. 9D depicts an extended random primer from FIG. 9C coupled to a nucleic acid capture probe.

FIG. 9E depicts the extended random primer and nucleic acid capture probe of FIG. 9D following extension of the nucleic acid capture probe using the extended random primer as a template and further extension of the extended random primer using the nucleic acid capture probe as a template.

FIG. 10A depicts circularization probes that are coupled to each instance of a target nucleic acid of a DNA molecule, as well as a blown up view of a circularization probe coupled to an instance of the target nucleic acid.

FIG. 10B depicts the circularization probe and instance of the target nucleic acid of FIG. 10A following extension of the circularization probe using the target nucleic acid as a template.

FIG. 10C depicts the extended circularization probe of FIG. 10B, wherein the extended circularization probe has been circularized through ligation.

FIG. 10D depicts the circularization probe of FIG. 10C following linearization and hybridization to a nucleic acid capture probe.

FIG. 10E depicts the circularization probe and nucleic acid capture probe of FIG. 10D following extension of the circularization probe using the nucleic acid capture probe as a template and extension of the nucleic acid capture probe using the circularization probe as a template.

FIG. 10F depicts the extended capture probe of FIG. 10E following further extension using a template switch oligonucleotide as a template.

FIG. 11A depicts circularization probes that are coupled to each instance of a target nucleic acid of a DNA molecule, as well as a blown up view of a circularization probe coupled to an instance of the target nucleic acid.

FIG. 11B depicts the circularization probe and instance of the target nucleic acid of FIG. 11A following extension of the circularization probe using the target nucleic acid as a template.

FIG. 11C depicts the extended circularization probe of FIG. 11B, wherein the extended circularization probe has been circularized through ligation.

FIG. 11D depicts the circularization probe of FIG. 11C following linearization and hybridization to a nucleic acid capture probe.

FIG. 11E depicts the circularization probe and nucleic acid capture probe of FIG. 11D following extension of the circularization probe using the nucleic acid capture probe as a template and extension of the nucleic acid capture probe using the circularization probe as a template.

FIG. 12A illustrates a surface with a capture probe and a barcode primer disposed thereon, in accordance with some embodiments.

FIG. 12B illustrates a messenger RNA (mRNA) molecule with a poly(A) tail hybridized to a poly(T) tail of the capture probe shown in FIG. 12A, in accordance with some embodiments.

FIG. 12C illustrates cDNA generated by extension of a capture probe using an mRNA molecule as a template.

FIG. 12D illustrates a template switching oligonucleotide (TSO) that is hybridized to a 3′ overhang of the cDNA of FIG. 12C and ligated to the mRNA molecule.

FIG. 12E illustrates the complementary deoxyribonucleic acid (cDNA) sequence extended using a template switch oligonucleotide as a template, in accordance with some embodiments.

FIG. 12F illustrates a cDNA sequence with a 3′ sequence that is a complement of a template switch oligonucleotide, in accordance with some embodiments.

FIG. 12G illustrates the nucleic acid of FIG. 12F hybridizing to a portion of a barcode primer, in accordance with some embodiments.

FIG. 12H illustrates a first product strand generated by extending the nucleic acid of FIG. 12F using the barcode primer as a template, and a second product strand generated by extending the barcode primer using the nucleic acid of FIG. 12F as a template, in accordance with some embodiments.

FIG. 12I illustrates the first and second product strands of FIG. 12H following dehybridization.

FIG. 12J illustrates the first product strand hybridizing to an additional barcode primer on the surface, in accordance with some embodiments.

FIG. 12K illustrates an additional second product strand generated by extending the additional barcode primer using the first product strand as a template, in accordance with some embodiments.

FIG. 13A illustrates circularization probes coupled to sequences of interest in a DNA molecule.

FIG. 13B illustrates the circularization probes of FIG. 13A following a circularization step in which ends of the circularization probes are coupled through ligation.

FIG. 14A illustrates first and second oligonucleotide probes coupled to each instance of a sequence of interest in a DNA molecule.

FIG. 14B illustrates the oligonucleotide probes of FIG. 14A following ligation of the first oligonucleotide probes to the second oligonucleotide probes, thereby forming ligation products.

FIG. 14C illustrates a ligation product of FIG. 14B coupled to a nucleic acid capture probe.

FIG. 15A shows a portion of another embodiment of a fluidic device including a sealable aperture.

FIG. 15B shows a method of trapping biological components in a portion of a fluidic device, according to some embodiments.

FIGS. 16A-16C show an example of a method for forming a compartment using a stimulus-responsive polymer. FIG. 16A illustrates a cross-sectional side view of a fluidic device showing the stimulus-responsive polymer disposed on a top layer of the fluidic device in a retracted state. FIG. 16B illustrates a bottom view of the fluidic device showing the stimulus-responsive polymer. FIG. 16C illustrates a cross-sectional side view of a fluidic device showing the stimulus-responsive polymer in an expanded closed state enclosing a biological material along with surface bound discrete sites that include nucleic acid capture probes.

### DETAILED DESCRIPTION

The present disclosure provides methods for determining copy number variation and genomic location for nucleic acid sequences of interest. Copy number variants measured by the present methods may include a number of instances of a gene, a number of instances of a repeat sequence, a number of instances of a noncoding sequence, or a combination thereof of a DNA molecule or genome. A copy number variant measured by the present methods may include a short tandem repeat (e.g., an about 2 to 10 nucleotide sequence), a mini-satellite (e.g., an about 10-60 nucleotide sequence), a long tandem repeat (e.g., a sequence of about 100 nucleotides or greater), or a combination thereof. Copy number variants can be measured within a particular locus, on a particular chromosome, or throughout a full genome. The disclosed methods may similarly be applied to vectors or plasmids carried by a cell.

The presently disclosed methods may determine the copy number and location of target nucleic acids in genomic DNA, plasmid DNA, vector DNA, extrachromosomal circular DNA (eccDNA), viral DNA, as well as combinations thereof. A method may measure the location and/or number of instances of a target sequence within a single DNA molecule or within all DNA present in a cell. In particular cases, the DNA can be genomic DNA. The target nucleic acid sequence may include a knock-in gene, a transposable gene or genetic element, a replicated gene or genetic element, or a similar sequence of a DNA molecule. A DNA molecule can include multiple instances of the target nucleic acid sequence, each instance being adjacent to a different flanking DNA sequence from the DNA molecule. The flanking DNA sequence may be of sufficient length to associate the target nucleic acid sequence with a unique location in the DNA molecule. A DNA molecule may alternatively include no instances of the target nucleic acid sequence, in which case a disclosed method may indicate the absence of the target nucleic acid sequence of the DNA molecule.

The presently disclosed methods extend copy number variation and genomic location measurements to single cells. A method may include compartmentalizing a single cell, for example by synthesizing one or more polymeric chambers to enclose the single cell. The one or more polymeric chambers may be impermeable to the cell and permeable to media, nutrients, and reagents, such that the cell may remain viable while it is enclosed within the chamber. The cell may be subjected to one or more forms of analysis while it is enclosed within the one or more chambers, including brightfield imaging, surface marker identification, soluble factor (e.g., cytokine) secretion detection, and the like. The cell may also be lysed or permeabilized while it is enclosed within the one or more chambers to release one or more cellular nucleic acids for analysis. The one or more chambers may be impermeable to the one or more cellular nucleic acids, and may thereby prevent the one or more cellular nucleic acids from exiting the one or more chambers. The one or more cellular nucleic acids may then be analyzed as described herein.

Examples of applications of the disclosed methods include cancer diagnostics and therapeutic cell (e.g., engineered T cell) analysis. Regarding cancer, many forms of cancer progression are associated with gene copy number alteration, which can involve gain or loss of DNA sections, as well as aneuploidy, which can involve a change in chromosome copy number. Such changes can drive tumor initiation, progression, metastasis, and therapeutic resistance. Examples include amplification of growth genes such as HER2 and EGFR, as well as deletions of cell cycle regulators such as CDKN2A, which can dysregulate cancer cell growth and proliferation. A method disclosed herein may detect gene amplification, deletion, polysomy, monosomy, aneuploidy, loss of heterozygosity, as well as combinations thereof in cancer cells. The cancer cells may be derived from tumor cells, circulating tumor cells, cell-free tumor DNA, bone marrow aspirates, cytology specimens, fresh or frozen tissue, formalin-fixed paraffin-embedded samples, as well as combinations thereof from a subject.

The disclosed methods may analyze samples at a single-cell level, and may thereby enable detection of low frequency advanced stage cancer cells. Cancer heterogeneity limits the accuracy of many forms of cancer diagnostics. Within individual subjects and even within individual tumors, cancer cells often exhibit a range of somatic mutations associated with different degrees of cancer progression and cancer cell behavior. Small subsets of highly mutated cells can be responsible for metastasis and cancer progression in subjects. However, bulk genomic analyses of cancer samples, which often return copy number averages, can miss highly mutated, advanced cancer stage cells, and can thereby mischaracterize cancer stage in subjects. The presently disclosed methods address this issue by analyzing copy number at a single cell level, and returning such information in tandem with additional cellular data such as mRNA expression, surface protein expression, protein secretion, proliferation, and morphology to accurately define cancer cell behavior and state.

As a further example, the disclosed methods can also be applied to therapeutic cell engineering and manufacture. Engineered cellular therapies-including chimeric antigen receptor (CAR) T cells, T-cell receptor (TCR) engineered lymphocytes, NK cells, and macrophages—can be generated by introducing one or more exogenous nucleic acid constructs into cells through transfection or viral transduction, through transposition, and through genome editing systems such as CRISPR-Cas nuclease cleavage followed by recombination. Cell populations generated using such methods can exhibit variation in transgene copy number as well as genomic rearrangements and structural alterations introduced during genome editing or vector integration. However, therapeutic efficacy and safety can depend on the genetic composition of individual engineered cells. Moreover, therapeutic efficacy and safety can depend on the proportions of cells with desired transgene integration sites and/or copy numbers, as well as additional factors that are not elucidated by integration site and copy number analyses alone. Bulk analysis methods, low throughput single cell analysis methods, and single cell analysis methods that cannot connect copy number and integration site measurements to other cellular characteristics can thus be incapable of assessing therapeutic formulation efficacy.

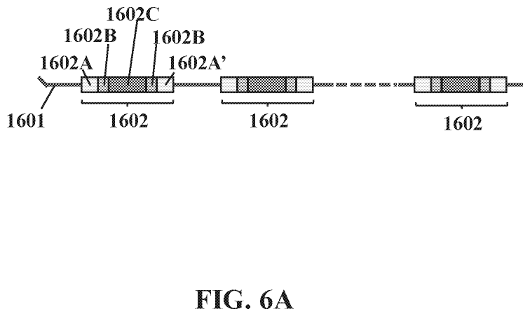

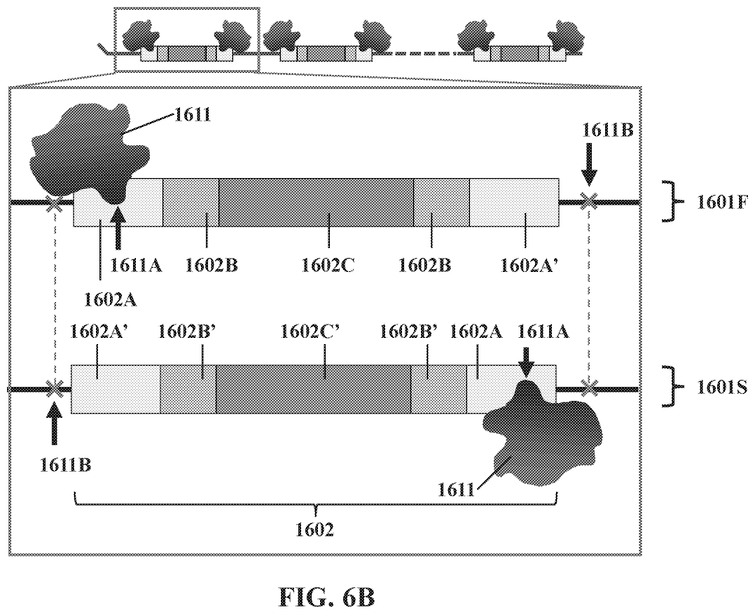

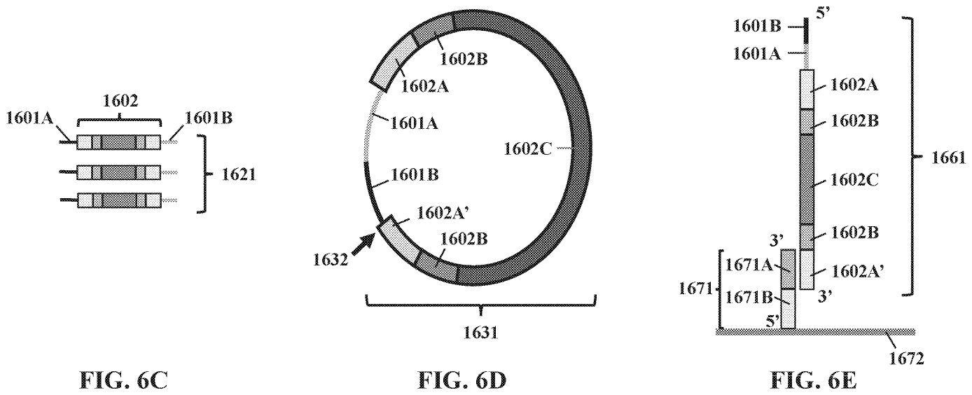

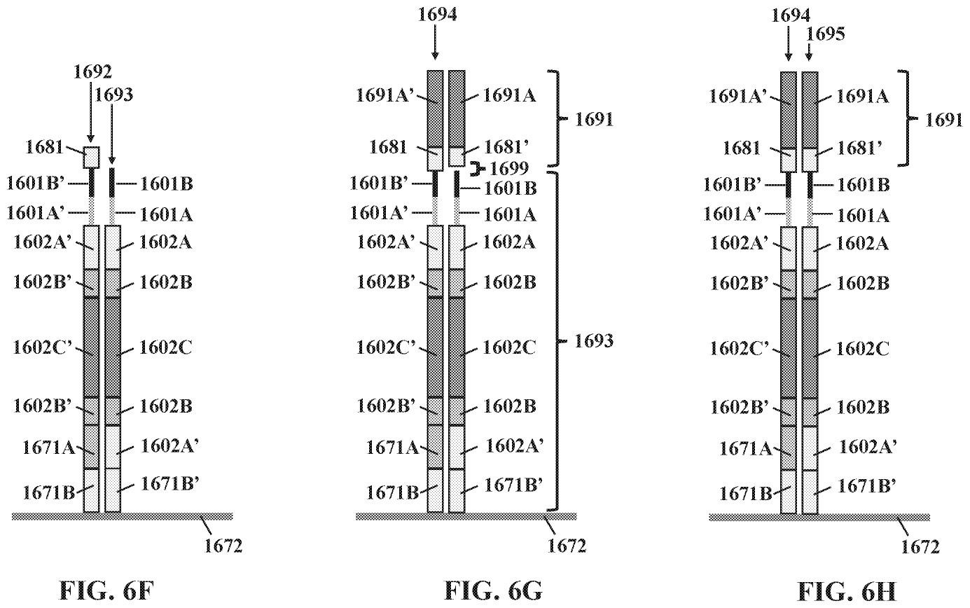

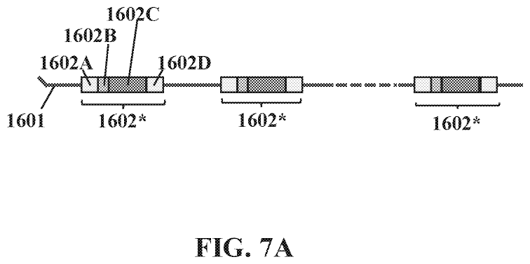

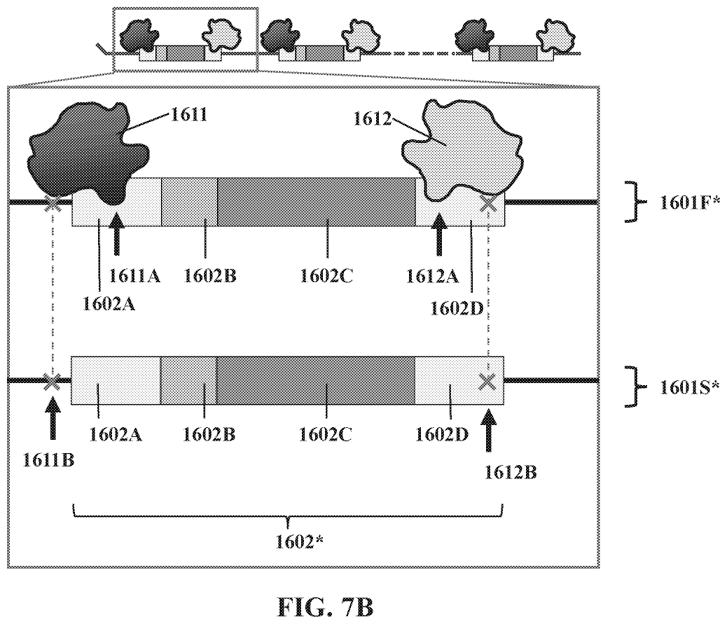

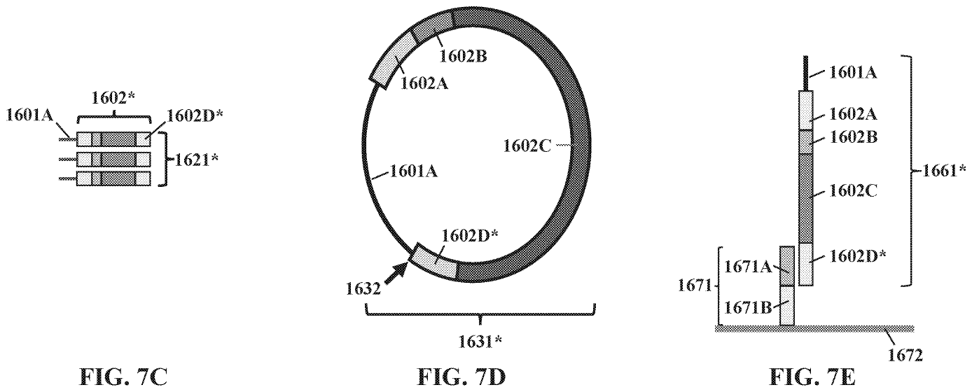

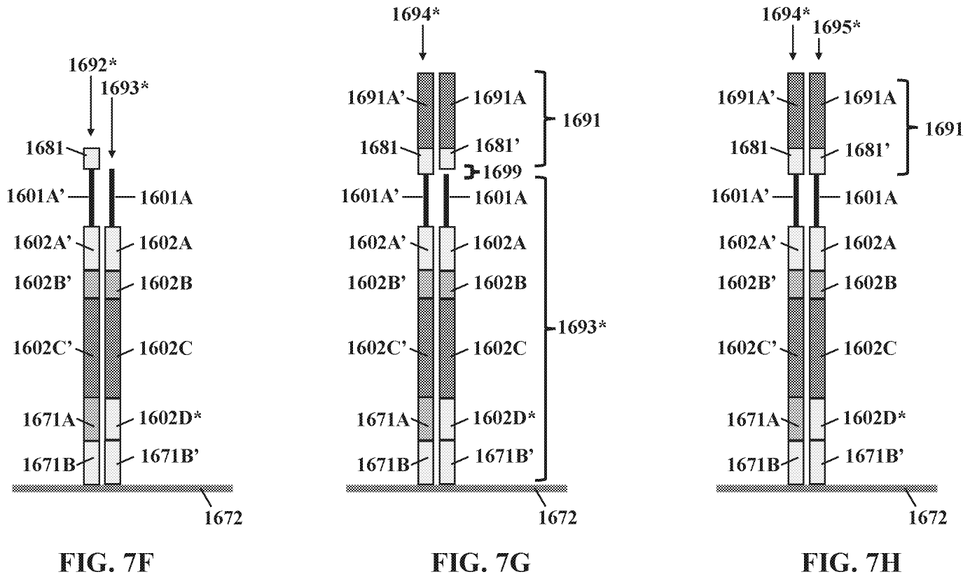

In some cases, a disclosed method measures the number of instances of a sequence in a genome, the location(s) of the sequence in the genome, the number of instances of the sequence in vectors, the location(s) of the sequence in the vectors, the number of instances of the sequence in plasmids, the location(s) of the sequence in the plasmids, the number of instances of the sequence in extrachromosomal circular DNA, the location(s) of the sequence in the extrachromosomal circular DNA, the number of instances of the sequence in viral DNA, the location(s) of the sequence in the viral DNA, or a combination thereof. Examples of such methods are outlined in FIGS. 6A-G and FIGS. 7A-G, which are described elsewhere herein. In some embodiments, a method may include inputting a cell into a fluidic device, wherein the cell comprises a DNA molecule; cleaving the DNA molecule with first and second restriction enzymes, wherein the cleaving generates a nucleic acid fragment that includes: i) the first handle or a portion of the first handle, ii) the target nucleic acid sequence, and iii) a flanking DNA sequence from the DNA molecule disposed at an end of the nucleic acid fragment; hybridizing the nucleic acid fragment or an amplicon generated from the nucleic acid fragment to a nucleic acid capture probe; extending the nucleic acid capture probe using at least a portion of the nucleic acid fragment or the amplicon as a template, thereby generating an extended nucleic acid capture probe comprising a complement of the target nucleic acid sequence and a complement of the flanking DNA sequence; and sequencing the nucleic acid capture probe or a nucleic acid generated from the nucleic acid capture probe.

While the flanking DNA sequence will often be sufficient for normalizing counts of the target nucleic acid sequence, the target nucleic acid sequence can optionally be proximal to one or more unique molecular identifiers that prevent duplicate counts of an individual instance of the target sequence. The one or more unique molecular identifiers may be disposed between the sites cleaved by the first and second restriction enzymes, and accordingly may be present in the nucleic acid fragment generated during cleavage. An example of a DNA molecule with a unique molecular identifier is provided in FIG. 7B, which includes, in order, a first restriction enzyme cleavage site 1611B, a first handle 1602A, a unique molecular identifier 1602B, a target nucleic acid sequence 1602C, a second handle 1602D, and a second restriction enzyme cleavage site 1612B disposed within the second handle. As used herein, the term handle can denote a portion of a nucleic acid that includes one or more of a capturable sequence, a barcode sequence, a restriction enzyme binding site, a sequencing adaptor, a primer binding site, or a combination thereof. The term barcode can refer to a short section of nucleotides that can be used to identify a characteristic of a sample (e.g., a source or location). In some cases, a barcode can also be in the form of a primer.

As is described elsewhere herein for FIG. 7B, the cleaving can include hybridizing the first restriction enzyme 1611 to a first restriction enzyme binding site 1611A of the DNA molecule and hybridizing the second restriction enzyme 1612 to a second restriction enzyme binding site 1612A of the DNA molecule, wherein the first handle 1602A or the portion of the first handle 1602A and the target nucleic acid sequence 1602C are disposed between the first restriction enzyme binding site 1611A and the second restriction enzyme binding site 1612A within the DNA molecule 1601. The first and second restriction enzymes may be identical (for example as depicted in FIG. 6B, in which two copies of the restriction enzyme 1611 are coupled to restriction enzyme binding sites 1611A on opposite end regions of a sequence of interest 1602) or different (for example as depicted in FIG. 7B, in which a first restriction enzyme 1611 is coupled to a first restriction enzyme binding site 1611A at a first end region of a sequence of interest 1602 and a second restriction enzyme 1612 is coupled to a second restriction enzyme binding site 1612A at a second end region of the sequence of interest 1602). As used herein, the term ‘end region’ denotes a terminal portion of a nucleic acid or a nucleic acid sequence. For example, a 1000 base pair sequence of interest may include a first end region that includes the first about 50 base pairs of the sequence of interest, and the sequence of interest may include a second end region that includes the final about 50 base pairs of the sequence of interest. The first end region and second end region are at opposing end regions of the sequence of interest 1602. In some cases, only one of the first and second restriction enzymes generates a flanking DNA sequence (for example as depicted in FIGS. 7B-C, in which the first restriction enzyme 1611 cleaves at a position 1611B outside of a sequence of interest 1602 to generate a flanking DNA sequence 1601A, and a second restriction enzyme 1612 cleaves at a position 1612B inside of the sequence of interest 1602 and does not generate a flanking DNA sequence).

The first restriction enzyme may be a type II restriction endonuclease. Many type II restriction endonucleases cleave nucleic acids at a position outside of their recognition sites. A type II restriction endonuclease may bind to a specific nucleotide recognition sequence and cleave at a position 10, 20, 30, or more nucleotides upstream or downstream of that sequence. Accordingly, a type II restriction endonuclease may bind to a known sequence within a knock-in gene (or similar target nucleic acid) but cleave at a position outside of the knock-in gene to collect a flanking DNA sequence outside of the knock-in gene. The first restriction enzyme and the second restriction enzyme may each be a type II restriction enzyme. Examples of type IIS restriction endonucleases include AcuI, AlwI, BaeI, BbsI, BbsI-HF, BbvI, BccI, BceAI, BcgI, BciVI, BcoDI, BfuAI, BmrI, BpmI, BpuEI, BsaXI, BseRI, BsgI, BsmAI, BsmFI, BsmI, BspCNI, BspMI, BspQI, BsrDI, BsrI, BtgZI, BtsCI, BtsI-v2, IIT, IIT, Earl, EciI, Eco57I, Esp3I, FauI, FokI, HgaI, HphI, HpyAV, MboII, MlyI, MmeI, MnlI, NmeAIII, PaqCI, PleI, SapI, and SfaNI. Further examples of Type IIS restriction endonucleases are provided in Pingoud et al., Nucleic Acids Research, 2014; 42(12):7489-7527 and Pingoud and Jeltsch, Nucleic Acids Research, 2001; 29(18):3705-3727.

The first restriction enzyme and/or the second restriction enzyme may also be a programmable nuclease such as a Csm1, Cas9, Cas12a, cpf1, an engineered argonaute protein, or a combination thereof. The programmable nuclease may include a guide nucleic acid targeted to the target nucleic acid, a barcode sequence, a handle (e.g., 1602A or 1602D in FIG. 7B) or a restriction enzyme binding site (e.g., 1611A or 1612A in FIG. 7B).

The first restriction enzyme binding site may be disposed within the first handle of the DNA molecule. However, the first restriction enzyme may cleave at a separate site outside of the first handle, but within surrounding DNA, to generate the flanking DNA sequence 1601A within a nucleic acid fragment (e.g., 1621 in FIG. 7C). The second restriction enzyme binding site may be disposed within a second handle on an opposite side of the target nucleic acid sequence as the first barcode sequence. The second restriction enzyme may cleave within the second handle or outside of the second handle. In some cases, the second restriction enzyme cleaves within the second handle. In other cases, the second restriction enzyme outside of the second handle. In particular cases, the first restriction enzyme binding site is disposed at or near to a first end of the first handle, the target nucleic acid sequence is adjacent to a second end of the first handle, and the first end of the first handle is opposite the second end of the first handle. As an example, a sequence of interest can include, in order, a first handle, a target nucleic acid sequence, and a second handle, wherein a first restriction enzyme binding site is disposed within the first handle and a second restriction enzyme binding site is disposed within the second handle, and wherein the first and second restriction enzyme binding sites are at or near outer edges of the sequence of interest. The restriction enzymes may each generate a flanking DNA sequence. For example, as depicted in FIGS. 6A-E, the nucleic acid fragment 1621 can include an additional flanking DNA sequence from the DNA molecule, wherein the flanking DNA sequence 1601A and the additional flanking DNA sequence 1601B are disposed at opposite ends of the nucleic acid fragment 1621.

As described above, the nucleic acid fragment generated by the restriction enzymes can include a second barcode or a portion of a second barcode sequence in addition to the first barcode or the portion of the first barcode. In some such cases, the nucleic acid fragment includes, in order, the flanking DNA sequence, the first barcode, the target nucleic acid sequence, and the portion of the second barcode. In other cases, the nucleic acid fragment includes, in order, the flanking DNA sequence, the first barcode, the target nucleic acid sequence, and the second barcode. As a further possibility, the nucleic acid fragment may include, in order, the first barcode, the target nucleic acid sequence, the second barcode, and the flanking DNA. Alternatively, the nucleic acid fragment may include, in order, the portion of the first barcode, the target nucleic acid sequence, the second barcode, and the flanking DNA sequence. As an additional example, the nucleic acid fragment can include, in order, the flanking DNA sequence, the first barcode sequence, the target nucleic acid sequence, the second barcode sequence, and an additional flanking DNA sequence from the DNA molecule.

The DNA molecule may be inside of a cell during the cleaving, in which cases the nucleic acid fragment can be released from the cell following the cleaving. For example, the first and second restriction enzymes may be delivered into the cell in a lipid particle, on a bead (uptaken, for example, through phagocytosis), through membrane poration (while the cell is alive or following fixation), or through another similar method known in the art. The cell may then be lysed to release the nucleic acid fragment following cleavage.

Alternatively, the DNA molecule may be outside of a cell during cleavage. For example, a cell containing the DNA molecule may be lysed before the first and second restriction enzymes couple to and cleave the DNA molecule. Following or during cell lysis (whether the lysis occurs before or after the cleavage), the method may include clearing nucleosomes and chromatin from the DNA molecule. Such clearance may reduce local viscosity and improve reagent access to the DNA molecule. Nucleosome and chromatin clearance may include an enzymatic treatment, for example DNase I or benzonase treatment, to digest DNA and dismantle chromatin structure. Nucleosome and chromatin clearance may also include treatment with a salt (e.g., 150-300 mM NaCl) and/or a mild detergent (e.g., Triton X-100 or NP-40) to solubilize and dissociate proteins from the DNA molecule.

Following cleavage, the nucleic acid fragment may couple to a nucleic acid capture probe. In many cases, the nucleic acid capture probe is coupled to a surface of the fluidic device, such as a top or bottom glass surface of a fluidic device channel. However, the nucleic acid capture probe may also be solubilized or coupled to an alternative substrate such as a bead. The nucleic acid capture probe can include a capture sequence that is configured to couple to a portion of the nucleic acid fragment. For example, the capture sequence can be configured to couple to the first or second handle or a portion of the first or second handle of the nucleic acid fragment. In specific cases, the first or second handle includes a polyA sequence that is configured to hybridize to a polyT sequence of the capture probe. However, the first handle, the portion of the first handle, the second handle, or a portion of the second handle of the nucleic acid fragment may couple to the nucleic acid capture probe.

The nucleic acid capture probe can also include one or more additional sequences such as a barcode or a sequencing adaptor. As depicted in FIG. 6E, the nucleic acid capture probe 1671 may be coupled to a surface 1672 through its 5′ end. The one or more additional sequences 1671B may be disposed between the capture sequence 1671A and the point of nucleic acid capture probe attachment to the surface. Examples of additional sequences that may be present in the nucleic acid capture probe include spatial barcodes, primer binding sites, unique molecular identifiers, restriction enzyme binding sites, suppressive or semi-suppressive PCR handles, sequencing adapters, and combinations thereof.

In particular embodiments disclosed herein, the nucleic acid capture probe is coupled to a surface of the fluidic device, and the nucleic acid capture probe includes a spatial barcode that is associated with a location of the nucleic acid capture probe along the surface of the fluidic device. In an exemplary application of this design, the cell may be compartmentalized. For example, a chamber may be synthesized around the cell and the nucleic acid capture probe, thereby confining the cell to a subspace that includes the nucleic acid capture probe. Alternatively or in addition thereto, the cell may be compartmentalized within a droplet, a well, or a pen. The cell may be incubated and/or analyzed while it is enclosed within the compartment (e.g., the chamber, droplet, well, or pen). For example, the cell's morphology, surface marker expression, secretions, and motility may be monitored as disclosed elsewhere herein. Then, the cell may be lysed, and a nucleic acid fragment containing the target nucleic acid can be cleaved from genomic DNA from the cell. The nucleic acid fragment may be inhibited or prevented from diffusing out of the compartment, and may thus diffuse to and then couple to the nucleic acid capture probe co-enclosed within the chamber. When nucleic acid capture probe and nucleic acid fragment are extended using one another as templates and eluted from the fluidic device, the resultant extended nucleic acids may be associated with the particular cell with which they were co-enclosed based on the spatial barcode on the extended nucleic acid capture probe or the complement of the spatial barcode on the extended nucleic acid fragment.

In general, the nucleic acid capture probe is extended using the nucleic acid fragment as a template and the nucleic acid fragment is extended using the nucleic acid capture probe as a template. However, in some cases (e.g., when the nucleic acid capture probe includes a 3′ modification that prevents its extension) only one of the nucleic acid capture probe and nucleic acid fragment is extended. Examples of extended nucleic acid capture probe 1692 and extended nucleic acid fragment 1693 are provided in FIGS. 6F-G and extended nucleic acid capture probe 1692* and extended nucleic acid fragment 1693* in FIGS. 7F-G, which are described elsewhere herein. In addition to the complements of the target nucleic acid sequence and flanking DNA sequence, the extended nucleic acid capture probe can include a complement of any other sequence present in the nucleic acid fragment, including the first handle, a portion of the first handle, the second handle, a portion of the second barcode, or a combination thereof.

The nucleic acid fragment (e.g., 1621 in FIG. 6C or 1621* in FIG. 7C) may optionally be amplified before it is coupled to the nucleic acid capture probe. The nucleic acid fragment may be circularized prior to the amplification, for example using Gibson assembly ligation by a CircLigase, or ligation of sticky ends generated by the first and second restriction enzymes. When the amplicon is circular, the amplicon may be converted into a linear amplicon through cleavage, and the linear amplicon can hybridize to the nucleic acid capture probe. Alternatively, the amplicon may be linear. For example, a linear amplicon may be generated from the circularized nucleic acid fragment using rolling circle amplification, and copies of the nucleic acid fragment within the amplicon, when present, may optionally be separated through cleavage.

### (ii) Example of Method—FIGS. 6A-H

An example of a DNA analysis method that utilizes cleavage to generate a nucleic acid fragment with a target nucleic acid is depicted in FIGS. 6A-G. FIG. 6A depicts genomic DNA 1601 that includes multiple instances of a sequence of interest 1602. It is worth noting that the method of FIGS. 6A-G may also be performed on other types of nucleic acids such as plasmids, vectors, viral DNA, extrachromosomal circular DNA, and the like. The sequence of interest 1602 may be a knock-in gene cassette that includes a gene (the target nucleic acid sequence) flanked at a first end by a first handle 1602A, flanked at a second end by a complement of the first handle 1602A′, and with optional unique molecular identifiers 1602B. In this example, each instance of the sequence of interest 1602 includes, in order, the first handle 1602A, an optional unique molecular identifier sequence 1602B that is unique to each instance of the sequence of interest 1602, a target nucleic acid sequence 1602C (e.g., a gene, a sequence encoding mRNA, a sequence encoding tRNA, a sequence encoding guide RNA, etc.), an additional optional unique molecular identifier sequence 1602B that is unique to each instance of the sequence of interest 1602, and a complement of the first handle 1602A′. While FIG. 6A depicts the sequence of interest 1602 as having complementary first handles 1602A and 1602A′, and unique molecular identifier sequences 1602B on each side of the target nucleic acid sequence 1602C, in alternative embodiments, the sequence of interest 1602 may include only one unique molecular identifier 1602B, no unique molecular identifier 1602B, different handles on opposite sides of the target nucleic acid sequence 1602C, only one handle (e.g., a first handle 1602A on a first side of the target nucleic acid sequence 1602C and no handle on the opposite side of the target nucleic acid sequence), or a combination thereof. In various embodiments, the handle can include a barcode having a sequence indicating the identity of the target nucleic acid sequence 1602C (e.g., a gene) or the sequence of interest 1602 where the barcode has a lower number of nucleotides than the target nucleic acid sequence 1602C or the sequence of interest 1600, allowing the identification process to be more efficient. In further embodiments, the handle 1602A can include a barcode sequence that encodes information about the target nucleic acid sequence 1602C or the sequence of interest 1602, such as the type of cell in which the target nucleic acid sequence 1602C or the sequence of interest 1602 is contained within.

As illustrated in FIG. 6B, the genomic DNA 1601 can be contacted with a restriction enzyme 1611 that is configured to bind to restriction enzyme binding sites 1611A within the sequence of interest 1602. The genomic DNA 1601 may be double-stranded or single-stranded during the restriction enzyme cleavage step depicted in FIG. 6B. In this example, the genomic DNA 1601 is double-stranded, and the restriction enzyme binding sites 1611A are located within the first handle 1602A of a first strand 1601F and the first handle 1602A of a second strand 1601S, wherein the second strand is complementary to the first strand. The restriction enzyme 1611 performs double-strand cleavage. It is worth noting that one or more restriction enzyme binding sites 1611A can alternatively be located on the same strand, at an end region of the first handle 1602A or outside of the handle, and at an alternative location within sequence of interest 1602. The end region can be an area proximate to the end of the first handle.

The restriction enzyme 1611 can be configured to cleave at a position that is spatially separated from the restriction enzyme binding site 1611A, where the cleavage site 1611B is outside of sequence 1602. The cleavage site 1611B may be a defined distance from the restriction enzyme binding site 1611A. More specifically, the restriction enzyme 1611 can be configured to cleave at a position 1611B that is about 10 to 20, about 10 to 30, about 10 to 40, about 10 to 50, about 10 to 60, about 10 to 80, about 20 to 30, about 20 to 40, about 20 to 50, about 20 to 60, about 20 to 80, about 30 to 40, about 30 to 50, about 30 to 60, about 30 to 80, about 40 to 60, about 40 to 80 nucleotides, or about 80 to 160 nucleotides from the restriction enzyme binding site 1611A, or about 4 to 8, about 4 to 16, about 4 to 24, about 4 to 32, about 4 to 40, about 4 to 60, about 8 to 16, about 8 to 24, about 8 to 32, about 8 to 40, about 8 to 60, about 16 to 24, about 16 to 32, about 16 to 40, about 16 to 60, about 24 to 32, about 24 to 40, about 24 to 60, about 32 to 60 nucleotides, or about 60 to 120 nucleotides beyond an end of the sequence of interest 1602.

As depicted in FIG. 6C, the restriction enzyme may cleave each instance of the sequence of interest 1602 along with flanking genomic DNA sequences 1601A and 1601B, thereby generating a group of nucleic acid fragments 1621. The flanking genomic DNA sequences can be used to map the location of a particular target sequence (e.g., copy number of the gene) within the genomic DNA sequence.

Moving to FIG. 6D, the nucleic acid fragment 1621 can be circularized to generate circularized nucleic acid fragments 1631 that include, in a clockwise direction, the first flanking genomic DNA sequence 1601A, a first handle 1602A, the optional unique molecular identifier 1602B, the target nucleic acid sequence 1602C, an optional unique molecular identifier 1602B, a complement of the first handle 1602A′, and the second flanking genomic DNA sequence 1601B. The circularized nucleic acid fragments 1631 can be double-stranded or single-stranded. The circularization step may include, for example, the use of a T4 DNA ligase, the use of CircLigase, Gibson assembly, ligation of compatible sticky ends, the use of a recombinase, or the use of a terminal deoxynucleotidyl transferase.

The circularized nucleic acid fragments 1631 can be coupled to a primer and amplified. The primer may bind to the first handle 1602A, to a portion of the target nucleic acid sequence 1602C, or to another sequence within the circularized nucleic acid fragments 1631. The amplification may alternatively use a random primer. The amplification may generate linear or circular amplicons. Circular amplicons may be linearized in a cleavage step. The linear amplicons can include a single copy of the circularized nucleic acid fragments 1631 or multiple copies of the circularized nucleic acid fragments 1631. For example, using a method such as rolling circle amplification, multiple copies of the circularized nucleic acid fragments 1631 can be generated along a single amplicon strand and then cleaved (e.g., with a Cas protein or other targeted nuclease) to generate linear amplicons 1661. Linear amplicons that contain single copies of the circularized nucleic acid fragments 1631 may be generated using two primers with opposite directions along the circularized nucleic acid fragments 1631. Primer binding and cleavage sites may be selected so that the first and second flanking DNA sequences 1601A, 1601B are disposed on the same side of a linear amplicon. For example, circular amplicons may be cleaved at an end of a first handle 1602A, indicated as 1632 in FIG. 6D.

As depicted in FIG. 6E, a linear amplicon 1661 may be captured on a nucleic acid capture probe 1671. The nucleic acid capture probe 1671 may optionally be coupled to a surface 1672, such as the surface of a bead or a surface of a channel within a fluidic device. The nucleic acid capture probe 1671 may include a capture sequence 1671A configured to hybridize to a portion the linear amplicon 1661 such as the complement of the first handle 1602A′ or a portion of the complement of the first handle 1602A′. The first handle 1602A may comprise a poly A sequence configured to hybridize to a poly T capture sequence 1671A on the capture probe 1671. The capture probe 1671 may comprise one or more additional sequences 1671B, such as a spatial barcode, a primer binding site, a unique molecular identifier, a restriction enzyme binding site, a suppressive or semi-suppressive PCR handle, or a combination thereof.

The nucleic acid capture probe 1671 can be extended using a portion of the amplicon 1661 as a template and the amplicon can be extended using a portion of the nucleic acid capture probe as a template. The extension may be performed, for example, with a DNA polymerase or a reverse transcriptase. Resultant extended nucleic acids are depicted in FIG. 6F. The extended nucleic acid capture probe 1692 may include, in order, the one or more additional sequences 1671B, the capture sequence 1671A, a complement of the unique molecular identifier sequence 1602B′, a complement of the target nucleic acid sequence 1602C′, a complement of the unique molecular identifier sequence 1602B′, a complement of the handle 1602A′, a complement of the first flanking genomic DNA sequence 1601A′, a complement of the second flanking genomic DNA sequence 1601B′, and optionally an overhang sequence 1681. For example, the extension of the nucleic acid capture probe may be performed with a Taq polymerase that generates an adenosine overhang at a 3′ end of the extended nucleic acid capture probe 1692 or a reverse transcriptase that generates an oligocytosine overhang at a 3′ end of the extended nucleic acid capture probe 1692. The extended amplicon 1693 can include, in order, a complement of the one or more additional sequences 1671B′, the complement of the first handle 1602A′, the unique molecular identifier 1602B, the target nucleic acid sequence 1602C, the unique molecular identifier 1602B, the first handle 1602A, the first flanking DNA sequence 1601A, and the second flanking DNA sequence 1601B.

When the overhang sequence 1681 is present, the extended capture probe may optionally be hybridized with template-switch oligonucleotide 1691 and then extended using at least a portion of template-switch oligonucleotide 1691 as template. The template-switch oligonucleotide 1691 can include a complement of the overhang sequence 1681′ and a template-switch oligonucleotide sequence 1691A. As depicted in FIG. 6G, further extension of the extended nucleic acid capture probe using the template-switch oligonucleotide 1691 as a template may add a complement of the template-switch oligonucleotide sequence 1691A′ to the 3′ end of the further extended nucleic acid capture probe 1694. The template-switch oligonucleotide 1691 is not attached to the extended amplicon 1693, and is separated from the extended amplicon 1693 by a gap 1699. As shown in FIG. 6H, the template-switch oligonucleotide 1691 may also be extended using the further extended nucleic acid capture probe 1694 as a template, thereby generating an extended template-switch oligonucleotide 1695 that includes a copy of the extended nucleic acid capture probe onto the 3′ end of the template-switch oligonucleotide. In particular, the template-switch oligonucleotide may be extended using a reverse transcriptase or DNA polymerase with strand-displacement activity, in which cases the reverse transcriptase or DNA polymerase may unhybridize the extended amplicon 1693 from the extended nucleic acid capture probe 1692 as the template-switch oligonucleotide 1691 is extended using the extended nucleic acid capture probe 1692 as a template. As an alternative to this template-switching step, a sequence (e.g., a primer sequence or an adapter sequence) may instead be ligated to the 3′ end of the extended capture probe and/or extended amplicon.

The extended nucleic acid capture probe 1692, further extended nucleic acid capture probe 1694, the extended amplicon 1693, and/or the extended template-switch oligonucleotide 1691* may then be sequenced. The number of instances of the sequence of interest 1602 can then be calculated as the number of target nucleic acid sequence reads with unique flanking genomic DNA sequences (1601A and/or 1601B), unique molecular identifier sequences (1602B), or a combination thereof. When the flanking genomic DNA sequences (1601A and/or 1601B) cannot be uniquely referenced to a location in a genome, for example when the flanking genomic DNA sequences (1601A and/or 1601B) map to tandem repeats, read counts may be normalized against a unique molecular identifier (1602B). The location(s) of each instance of the sequence of interest 1602 can be determined by referencing the flanking genomic DNA sequences (1601A and/or 1601B) against a full genomic sequence of the organism from which the genomic DNA 1601 was obtained.

(iii) Additional Example of Method—FIGS. 7A-H

A similar method for determining copy number variation and/or the location(s) of a target nucleic acid sequence within a genome is depicted in FIGS. 7A-G. As shown in FIG. 7A, genomic DNA 1601 can include multiple instances of a sequence of interest 1602*. In this example, each instance of the sequence of interest 1602* includes a first handle 1602A, a unique molecular identifier 1602B, a target nucleic acid sequence 1602C, and a second handle 1602D that is different than the first handle 1602A. It is again worth noting that the method of FIGS. 7A-G may also be performed on other types of nucleic acids such as plasmids, vectors, viral DNA, extrachromosomal circular DNA, and the like.

Moving to FIG. 7B, the genomic DNA 1601 can be contacted with a first restriction enzyme 1611 configured to bind to a first restriction enzyme binding site 1611A within the first handle 1602A and a second restriction enzyme 1612 configured to bind to a second restriction enzyme binding site 1612A within the second handle 1602D. In this example, the first restriction enzyme 1611 and second restriction enzyme 1612 couple to the same strand 1601F* of the genomic DNA 1601. The genomic DNA 1601 may optionally be double-stranded, wherein the genomic DNA 1601 includes a second strand 1601S* that is complementary to the first strand 1601F*. The first restriction enzyme 1611 can be configured to cleave at a position 1611B outside of the first handle 1602A and within surrounding genomic DNA 1601. In this example, the second restriction enzyme 1612 cuts at a position 1612B within the second handle 1602D. However, the second restriction enzyme 1612 can alternatively cut at the boundary between the second handle 1602D, and surrounding genomic DNA 1601, or outside of the second handle 1602D and within flanking genomic DNA.

Each instance of the sequence of interest 1602* is cleaved from the genomic DNA 1601 by the first and second restriction enzymes 1611 and 1612, generating a group of nucleic acid fragments 1621*, which are depicted in FIG. 7C. Each nucleic acid fragment 1621* includes, in order, a flanking DNA sequence 1601A, the first handle 1602A, the unique molecular identifier 1602B, the target nucleic acid sequence 1602C, and a portion of the second handle 1602D*.

The nucleic acid fragments may optionally be circularized (as depicted in FIG. 7D), amplified, re-linearized, and fragmented as described for FIG. 6D. However, the nucleic acid fragments 1621* may also be directly bound to nucleic acid capture probes 1671 (as depicted in FIG. 7E) without prior circularization and/or amplification steps.

FIG. 7E illustrates linear amplicons 1661* (or, alternatively, the nucleic acid fragments 1621*) captured on a nucleic acid capture probe 1671. While in this example nucleic acid fragment 1621* is identical to linear amplicon 1661*, the linear amplicon 1661* may be different from the nucleic acid fragment 1621*. For example, the linear amplicon could be generated by cleaving an amplicon at the junction of the first handle 1602A and the unique molecular identifier 1602B to generate a linear amplicon that included, in order, the first handle 1602A, the flanking DNA sequence 1601A, the portion of the second handle 1602D*, and the target nucleic acid sequence 1602C. The nucleic acid capture probe may optionally be coupled to a surface 1672. The nucleic acid capture probe may include a capture sequence 1671A configured to hybridize to at least a portion of the second handle 1602D*. The capture probe may include one or more additional sequences 1671B, such as a spatial barcode, a primer binding site, a unique molecular identifier, a restriction enzyme binding site, a suppressive or semi-suppressive PCR handle, a sequencing handle, or a combination thereof.

In a concerted step, the nucleic acid capture probe 1671 may be extended using the linear amplicon 1661* as a template and the amplicon 1661* can be extended using the nucleic acid capture probe 1671 as a template. The products of this extension step are shown in FIG. 7F. The extended nucleic acid capture probe 1692* can include, in order, the one or more additional sequences 1671B, the capture sequence 1671A, a complement of the target nucleic acid sequence 1602C′, a complement of the unique molecular identifier sequence 1602B′, a complement of the first handle 1602A′, a complement of the first flanking genomic DNA sequence 1601A′, and optionally an overhang sequence 1681. The extended amplicon 1693* can include, in order, a complement of the one or more additional sequences 1671B′, the portion of the second handle 1602D*, the target nucleic acid sequence 1602C, the unique molecular identifier sequence 1602B, the first handle 1602A, and the flanking DNA sequence 1601A.

The extended nucleic acid capture probe 1692* can optionally undergo a further extension using at least a portion of the template switch oligonucleotide 1691 as a template (as shown in FIG. 7G and as described for FIG. 6G) to incorporate a complement of a template-switch oligonucleotide sequence 1691A′ at the 3′ end to form the further extended nucleic acid capture probe 1694*. Following this extension, the template-switch oligonucleotide 1691 is not attached to the extended amplicon 1693*, and is separated from the extended amplicon 1693* by a gap 1699. As shown in FIG. 7H, the template-switch oligonucleotide 1691 may be hybridized to the further extended nucleic acid capture probe 1694* and then extended using the further extended nucleic acid capture probe 1694* as a template, for example with the use of a strand-displacing DNA polymerase to displace the extended amplicon 1693*, thereby generating an extended template-switch oligonucleotide 1695*.

### (iv) Optical Pooled Screening

Referring again to FIGS. 6D and 7D, in some methods, linear amplicons are generated using the circularized nucleic acid fragments 1631, 1631*. The linear amplicons may be multiple times the lengths of the circularized nucleic acid fragments 1631, 1631*, and can include multiple complements of the circularized nucleic acid fragments 1631, 1631*. For example, amplification may utilize a DNA polymerase with strong strand-displacement activity such as Phi29 DNA polymerase. The amplification may include rolling circle amplification. The resultant amplicons may include tens, hundreds, or thousands of complements of the circularized nucleic acid fragments 1631, 1631*. The amplicon may be generated inside of a biological material such as a cell or outside of the biological material (e.g., following lysis). When the process is performed inside of a compartment such as a hydrogel chamber, the amplicon may be confined to the compartment. For example, the amplicon may be larger than pores through walls in a hydrogel chamber, and may therefore be incapable of diffusing through the pores and out of the chamber. Instead of or in addition to generating nucleic acids for sequencing (e.g., instead of or in addition to following the methods of FIGS. 6E-H and 7E-H), the amplicon may be detected by coupling optically detectable probes to the amplicon and detecting the optically detectable probes. The optically detectable probes may include oligonucleotides that hybridize to sequences on the amplicon. The optically detectable probes may thus be specific for a particular amplicon. In some cases, one optical probe may couple to each complement of the sequence of interest. The optically detectable probes may include optically detectable moieties such as dyes or fluorophores. The optically detectable probes may be detected through imaging, for example by locating and counting each amplicon with optically detectable probes coupled thereto or by counting total light intensity or absorbance from a compartment.

### (v) Nucleic Acid Fragment Capture

In further aspects disclosed herein, the nucleic acid fragment 1621, 1621* can be captured on the nucleic acid capture probe 1671. In such cases, a sequence at or near a 3′ end of the nucleic acid fragment (e.g., 1602D* of nucleic acid fragment 1621* in FIG. 7C) can be configured to hybridize to a capture sequence 1671A of a nucleic acid capture probe 1671. In particular cases, for example, the target nucleic acid sequence 1602C or a portion of the target nucleic acid sequence 1602C can be configured to hybridize to a capture sequence 1671A of a nucleic acid capture probe 1671. Prior to capture, the nucleic acid fragment 1621, 1621* can optionally be linearly (e.g., using a single primer targeted to a single known sequence of the nucleic acid fragment 1621, 1621*) or exponentially (e.g., using a pair of primers in which one primer is targeted to the nucleic acid fragment 1621, 1621* and one primer is targeted to a complement of the nucleic acid fragment 1621, 1621*) amplified prior to capture.

Similarly, in some cases, the 5′ and 3′ ends of the nucleic acid fragment 1621, 1621* can be shifted by circularizing the nucleic acid fragment 1621, 1621* and then reopening the resultant circularized nucleic acid fragment 1631, 1631* at a different location. Such a method may dispose a complement or near complement of a capture sequence 1671A of a nucleic acid capture probe 1671 at or near a 3′ end of the circularized and reopened nucleic acid fragment, allowing the fragment to be captured on a nucleic acid capture probe and for a portion of the fragment to serve as a template for nucleic acid capture probe 1671 extension. The circularized and reopened fragment can be linearly or exponentially amplified prior to nucleic acid capture probe 1671 capture.

### Circularization Probes

### (i) General Overview of Method

Further disclosed herein are methods that utilize circularization probes to determine gene copy number variation. A circularization probe may be a linear nucleic acid that is configured to hybridize to two sequences on a single strand of a target nucleic acid. The circularization probe may contain primers at its 3′ and 5′ ends. A first primer at a 3′ end of the circularization probe may hybridize to a first sequence within the target nucleic acid and a second primer at a 5′ end of the circularization probe may hybridize to a second sequence that is upstream of the first sequence along the target nucleic acid. When the target nucleic acid is present within a sample, the first and second primers of the circularization probe may hybridize, respectively, to the first and second sequences in the target nucleic acid. The first primer may optionally be extended up to the second primer using the target nucleic acid as a template. The first and second primers may optionally also be ligated, converting the circularization probe into a circular nucleic acid, allowing non-ligated, linear circularization probes (circularization probes that did not hybridize to the target nucleic acid) to be selectively digested. Remaining circularization probes can be linearized (if circularized), coupled to nucleic acid capture probes, and analyzed as disclosed elsewhere herein.

A method for analyzing a DNA molecule with a circularization probe may include inputting a cell into a fluidic device, wherein the cell comprises the DNA molecule; hybridizing a circularization probe to the DNA molecule, wherein: i) a first primer sequence of the circularization probe hybridizes to a first handle on a first strand of the DNA molecule, ii) a second primer sequence of the circularization probe hybridizes to a second handle on the first strand of the DNA molecule, and iii) a target nucleic acid sequence and at least one unique molecular identifier sequence are disposed between the first handle and the second handle within the first strand of the DNA molecule; extending the first primer sequence using the target nucleic acid sequence and the at least one unique molecular identifier sequence as a template, thereby generating a 3′ extension coupled to the 3′ end of the first primer sequence; hybridizing the extended circularization probe or an amplicon generated therefrom to nucleic acid capture probes coupled to a surface of the fluidic device; extending the nucleic acid capture probe using the circularization probe or amplicon as a template, thereby generating an extended nucleic acid capture probe comprising a complement of the target nucleic acid sequence; and sequencing the nucleic acid capture probes or nucleic acids generated from the nucleic acid capture probes.

The method may further include circularizing the circularization probe, for example by ligating the 3′ extension to the second primer sequence. The ligase may be a T4 DNA ligase or a similar enzyme that selectively ligates broken strands along double stranded DNA. Accordingly, the ligase may only circularize circularization probes that are hybridized to the target nucleic acid, and may be inert to circularization probes that are not bound to the target nucleic acid. Following the ligation, circularization probes that were not hybridized to the target nucleic acid may be linear, and may optionally be removed through exonuclease digestion. The circularized circularization probes can optionally be amplified to generate linear or circular amplicons, which may optionally be cleaved and then coupled to nucleic acid capture probes as disclosed herein. Alternatively, the circularized circularization probes can be cleaved open and then coupled to nucleic acid capture probes. The circularization probe can further include an internal nucleic acid sequence connecting the first and second primers. As examples, the internal nucleic acid sequence may include an additional unique molecular identifier sequence; a restriction enzyme binding site, wherein the cleaving comprises binding a nuclease to the restriction enzyme binding site; a capturable sequence, wherein the hybridizing the linear amplicons to nucleic acid capture probes comprises hybridizing the capture sequence or a complement of the capture sequence to the nucleic acid capture probes; one or more barcode sequences; or a combination thereof. In some cases, the circularization probe internal nucleic acid sequence may include a restriction enzyme binding site downstream (3′) to a capturable sequence. Following ligation, circularized circularization probes may be cleaved at the restriction enzyme binding site, such that the capturable sequence is disposed at 3′ ends of the resulting linearized circularization probes. The capturable sequences of the linearized circularization probes may then hybridize to capture sequences on nucleic acid capture probes, facilitating nucleic acid capture probe extension using the linearized circularization probes as templates and linearized circularization probe extension using the nucleic acid capture probes as templates as disclosed herein.

As with other methods disclosed herein, the circularization probe may be coupled to the DNA molecule and circularized inside of the cell or following release of the DNA molecule from the cell. The DNA molecule may, for example, include genomic DNA, plasmid DNA, vector DNA, extrachromosomal circular DNA (eccDNA), viral DNA, or a combination thereof.

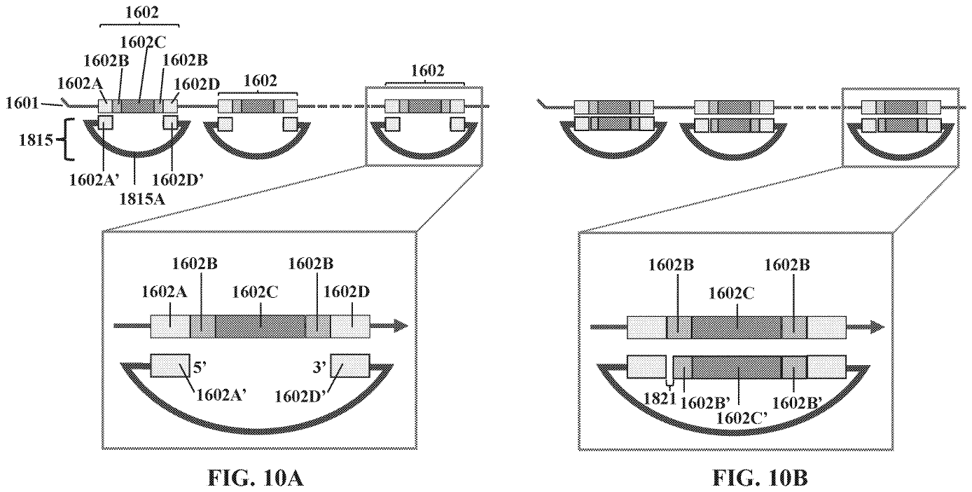

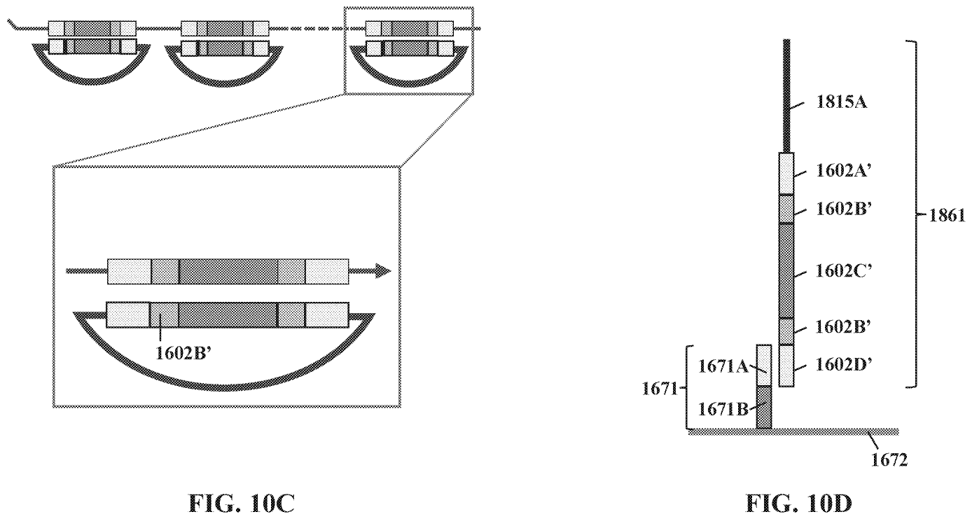

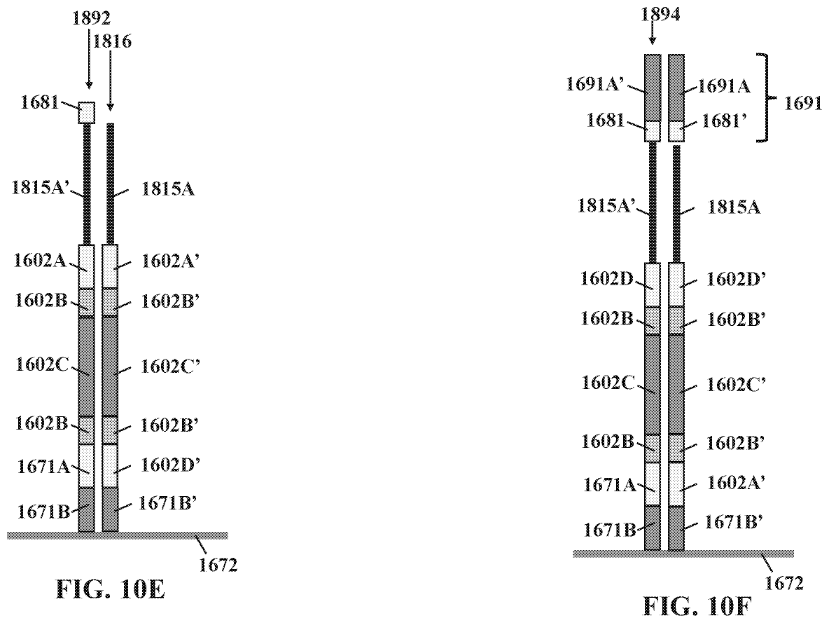

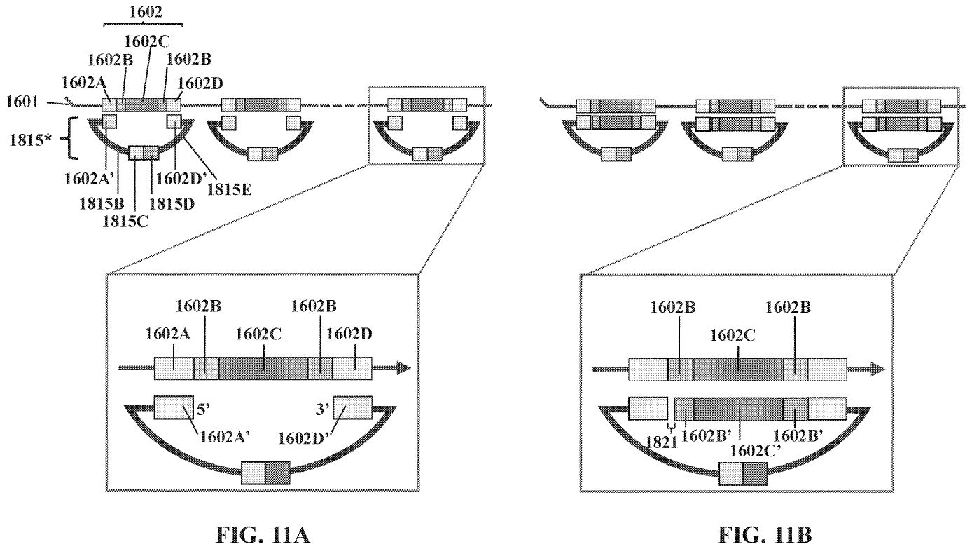

### (ii) Example of Method—FIGS. 10A-F

FIGS. 10A-F illustrate a circularization probe-based method for determining the number of instances of a sequence of interest 1602 in genomic DNA 1601. In this example, each sequence of interest includes, in order, a first handle 1602A, a unique molecular identifier sequence 1602B, a target nucleic acid sequence 1602C, a second unique molecular identifier sequence 1602B, and a second handle 1602D. However, the sequence of interest 1602 can alternatively include a single unique molecular identifier sequence 1602B rather than two unique molecular identifier sequences, as well as additional nucleic acid sequences between the first and second handles. The genomic DNA 1601 can be contacted to circularization probes 1815 that are targeted to the sequence of interest 1602. One circularization probe 1815 may couple to each instance of sequence of interest 1602.

The genomic DNA 1601 may be inside of a cell or released from a cell during circularization probe 1815 delivery and circularization. When the genomic DNA 1601 is inside of a cell, the circularization probes 1815 can be delivered into the cell using a known transfection method such as electroporation, microinjection, DEAE-dextran transfection, optical transfection, laserfection, calcium phosphate transfection, lipoparticle-based delivery, bead-based delivery, or star polymer-based delivery, or a known transduction method such as rAAV or lentiviral delivery. The cell may also be fixed, for example using formaldehyde, glutaraldehyde, methanol, ethanol, acetone, or a combination thereof; and permeabilized, for example using Triton X-100, Tween-20, saponin, methanol, ethanol, acetone, or a combination thereof. Circularization probes 1815 can then be delivered into the permeabilized cell through pores in the surface of the permeabilized cell. Alternatively, the cell can remain viable following circularization probes 1815 delivery and extension within the cell. Following circularization probe 1815 extension, the cell may be lysed or permeabilized to release the extended circularization probes (see FIG. 10B). Cell lysis prior to or following circularization probe 1815 extension can be performed, for example, using an endopeptidase such pepsin or proteinase K, light, electrical current, mechanical stress, a detergent such as polyethylene glycol tert-octylphenyl ether, polysorbate 80, polysorbate 20, N-lauroylsarcosine, sodium dodecyl sulfate, Triton X-100™, Tween-20™, bacteria-mediated lysis, or combinations thereof (e.g., as disclosed in Brown and Audet, J. R. Soc. Interface, 2008; 5:S131-S138; Danaeifar, Biotechnol Bioeng., 2022; 119(11):3007-3021, which is herein incorporated by reference for its disclosure on cell lysis and permeabilization). Chromatin, nucleosomes, and other DNA-binding proteins may be cleared from the genomic DNA 1601 as disclosed elsewhere herein.

As shown in FIG. 10A, circularization probes 1815 can be coupled to sequences of interest 1602 in genomic DNA 1601. In this example, the sequences of interest 1602 include, in order, first handle 1602A, unique molecular identifiers 1602B, target nucleic acid sequence 1602C, unique molecular identifiers 1602B, and second handle 1602D. Each circularization probe 1815 may have a complement to the first handle 1602A′ that hybridizes to the first handle 1602A of the sequence of interest 1602 and a complement to the second handle 1602D′ that hybridizes to the second handle 1602D of the sequence of interest 1602. In various embodiments, the complement to the second handle 1602D′ can function as a primer. The complement to the first and second handles may be coupled to an internal nucleic acid 1815A to form the circular probe 1815. The circular probe 1815 can include one or more unique molecular identifier sequences, handles, primer sequences, nuclease binding sites, capturable sequences (e.g., poly A sequences), and the like.

The circularization probes 1815 can then be extended using the sequences of interest 1602 as a template. As depicted in FIG. 10B, the complement to the second primer 1602D′ may be extended to generate, in order, a complement of the unique molecular identifier 1602B′, a complement of the target nucleic acid sequence 1602C′, and a complement of the unique molecular identifier 1602B′. Gaps 1821 may be present between the terminal portions of the complement of the unique molecular identifier 1602B′ and the complement to the first handle 1602A′ following extension. As shown in FIG. 10C, the gaps 1821 may be ligated closed, thereby circularizing the circularization probes. Following circularization probe closure, single-stranded nucleic acids may be digested to remove circularization probes that were not extended (e.g., using a sequence of interest 1602 as a template) or circularized.

The remaining circularization probes may then be opened, optionally amplified, and coupled to nucleic acid capture probes 1671 as shown in FIG. 10D. In this example, the second primer 1602D′ of the linearized circularization probe 1861 hybridizes to the capture sequences 1671A of the capture probes 1671. The capture probes 1671 also include one or more additional sequences 1671B as described elsewhere herein, and may be affixed to a surface 1672. However, while FIG. 10D depicts linearized circularization probes 1861 that were cleaved between the internal nucleic acid 1815A and second primer 1602D′, in alternate embodiments the capture probe may be cleaved in alternate locations, for example: (1) between the complement of the unique molecular identifier 1602B′ and the first primer 1602A′, in which case the first primers of the linearized circularization probes 1861 may hybridize to the nucleic acid capture probes 1671, or (2) within the internal nucleic acid 1815A, in which case a portion of the internal nucleic acid 1815A (e.g., a poly A sequence disposed within the internal nucleic acid 1815A) may hybridize to the nucleic acid capture probes 1671.

Next, the linearized circularization probes 1861 and nucleic acid capture probes 1671 may be extended to generate the extended linearized circularization probe 1816 and the extended nucleic acid capture probes 1892, respectively, as described for FIG. 10E. In this step, the linearized circularization probes 1861 may be extended using the one or more additional sequences 1671B of the nucleic acid capture probes 1671 as a template, thereby generating extended linearized circularization probes 1816 with complements of the one or more additional sequences 1671B′ adjacent to the first primer sequence 1602D′. The nucleic acid capture probes 1671 are extended using the linearized circularization probes 1861 as templates. The extended nucleic acid capture probes 1892 include, in order, the one or more additional sequences 1671B, the capture sequence 1671A, the unique molecular identifier 1602B, the target nucleic acid sequence 1602C, the unique molecular identifier sequence 1602B, the first handle 1602A, a complement of the internal nucleic acid of the circularization probe 1815A′, and an optional overhang 1681 as described elsewhere herein.

When the overhang 1681 is present, the extended nucleic acid capture probes may be further extended using a template-switch oligonucleotide 1691 as a template. The products of this extension step are illustrated in FIG. 10F. The template-switch oligonucleotide 1691 may include a complement of the overhang 1681′ that hybridizes to the overhang 1681 on the extended nucleic acid capture probe and a template-switch oligonucleotide sequence 1691A. A complement of the template-switch oligonucleotide sequence 1691A′ may be added to the end of the extended nucleic acid capture probe 1892 to form further extended nucleic acid capture probe 1894. Although not shown in FIG. 10F, the template-switch oligonucleotide 1691 may optionally be extended using the extended nucleic acid capture probe 1892 as a template.

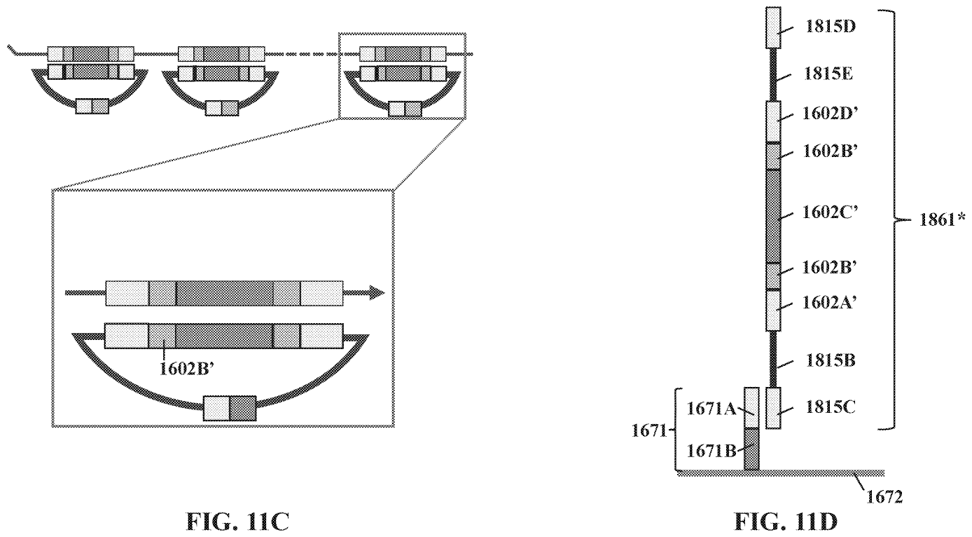

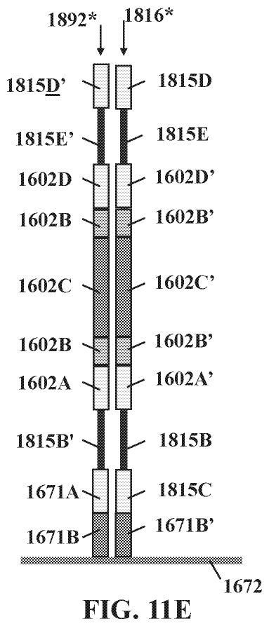

(iii) Additional Example of Method—FIGS. 11A-E

FIGS. 11A-E illustrate an additional method for using a circularization probe 1815* to determine the number of instances of a sequence of interest 1602 in genomic DNA 1601. Circularization probes 1815* may be coupled to the sequences of interest 1602 as described for FIG. 10A, wherein complements of the first handles 1602A′ of the circularization probes hybridize to first handles 1602A of the sequences of interest and complements of the second handles 1602D′ of the circularization probes hybridize to second handles 1602D of the sequences of interest. In this example, the circularization probes include, in order, complement to the first handle 1602A′, first internal sequence 1815B, first internal handle 1815C, second internal handle 1815D, second internal sequence 1815E, and the complement of the second handle 1602D′. As in FIG. 10A, the sequences of interest 1602 include, in order, first handle 1602A, unique molecular identifier 1602B, target nucleic acid 1602C, unique molecular identifiers 1602B, and second handle 1602D. The circularization probes 1815* are extended using the sequences of interest 1602 as templates to form the extended circularization probes as shown in FIG. 11B and ligated to close the loop as shown in FIG. 11C. As discussed elsewhere, in alternative aspects, the 5′ and 3′ ends of the circularization probe 1815* may couple directly adjacently along the sequence of interest 1602 (i.e., to consecutive nucleotides in the sequence of interest 1602) and may be ligated without an intervening extension step.

The extended circularization probes as shown in FIG. 11C are then cleaved open to form linearized circularization probes 1861*. In this example, the extended circularization probes of FIG. 11C are cleaved in between the first and second internal handles 1815C-D. Accordingly, each linearized circularization probe 1861* includes, in order, the first internal handle 1815C, the first internal sequence 1815B, a complement of the sequence of interest (1602A′, 1602B′, 1602C′, 1602B′, 1602D′), the second internal sequence 1815E, and the second internal handle 1815D. As depicted in FIG. 11D, the first internal handles 1815C of the linearized circularization probes 1861* may couple to the capture sequences 1671A of the nucleic acid capture probes 1671.

The linearized circularization probes 1861* and nucleic acid capture probes 1671 may then be extended as described for FIG. 10E. As depicted in FIG. 11E, the extended circularization probes 1816* can include, in order, a complement of the one or more additional sequences 1671B′, the first internal handle 1815C, the first internal sequence 1815B, a complement of the sequence of interest (1602A′, 1602B′, 1602C′, 1602B′, 1602D′), the second internal sequence 1815E, and the second internal handle 1815D. The extended nucleic acid capture probes 1892* can include, in order, the one or more additional sequence 1671B, the capture sequence 1671A, a complement of the first internal sequence 1815B′, the sequence of interest (1602A, 1602B, 1602C, 1602B, 1602D), a complement of the second internal sequence 1815E′, and a complement of the second internal handle 1815D′.

The extended circularization probes 1816* and/or the extended nucleic acid capture probes 1692* can be sequenced. The second internal handle 1815D and one or more additional sequences 1671B may each include a primer binding site or sequencing adaptor to facilitate the sequencing or an amplification step without further extension using a template-switch oligonucleotide as a template or a similar end-functionalization step such as ligation. As the circularization probe 1815* is extended using the sequence of interest 1602 as a template, the method may be used to identify mutations such as single nucleotide polymorphisms in individual instances of the sequence of interest.

A method for analyzing a DNA molecule with a circularization probe may alternatively include ligation of the first primer to the second primer without prior extension of the first primer. In such cases, the first and second primer sequences may hybridize to adjacent positions along the target nucleic acid. Such a method may include inputting a cell into a fluidic device, wherein the cell comprises the DNA molecule; hybridizing a circularization probe to the DNA molecule, wherein first and second primer sequences hybridize to adjacent positions within a target nucleic acid sequence on a strand of the DNA molecule; ligating the first primer sequence to the second primer sequence, thereby circularizing the circularization probe; digesting linear nucleic acids, wherein the digesting does not digest the circularized circularization probe; opening the circularized circularization probe; hybridizing the opened circularization probe to a nucleic acid capture probe coupled to a surface of the fluidic device; extending the nucleic acid capture probe using the opened circularization probe as a template, thereby generating an extended nucleic acid capture probe comprising a complement of the target nucleic acid sequence; and sequencing the nucleic acid capture probe or s nucleic acid generated from the nucleic acid capture probe.

(iv) Couple Circularization Probe Ends Directly Adjacently within Sequence of Interest

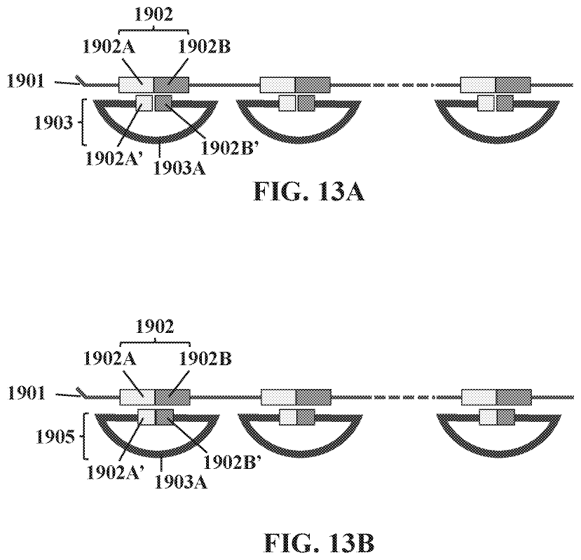

It is worth noting that in alternative aspects to those depicted in FIGS. 10A-C and FIGS. 11A-C, two portions of the circularization probe may couple directly adjacently to one another within the sequence of interest and directly ligated without an intervening extension step. An example of such a scheme is depicted in FIGS. 13A-B. As shown in FIG. 13A, circularization probes 1903 may couple to sequences of interest 1902 in a DNA molecule 1901. In this example, the sequences of interest 1902 include a first portion 1902A directly adjacent to a second portion 1902B. Each circularization probe 1903 may have a complement to the first portion 1902A′ (or a portion thereof) that hybridizes to the first portion 1902A of the sequence of interest 1902 and a complement to the second portion 1902B′ (or a portion thereof) that hybridizes to the second portion 1902B′ of the sequence of interest 1902. The circularization probe 1903 may further include an internal nucleic acid 1903A that connects the complement to the first portion 1902A′ and complement to the second portion 1902B′. The internal nucleic acid 1903A can include one or more barcodes, unique molecular identifiers, primer binding sites, or other sequences that can aid in handling and processing. The complement to the first portion 1902A′ and the complement to the second portion 1902B′ may couple directly adjacently within the sequence of interest 1902, such that terminal nucleotides of the complement to the first portion 1902A′ and the complement to the second portion 1902B′ are hybridized to nucleotides within the sequence of interest 1902 that are connected by a single phosphodiester group.

Next, as depicted in FIG. 13B, the complement to the first portion 1902A′ and the complement to the second portion 1902B′ may be ligated to convert the circularization probes 1903 into a circularized circularization probes 1905. During such a step, only circularization probes 1903 that are coupled to the sequence of interest 1902 may be ligated to form circularized circularization probes 1905. Non-circularized (i.e., non-ligated) circularization probes 1903 may then be digested, and the circularized circularization probes 1905 may be processed and analyzed as described elsewhere herein.

### (v) Tandem Oligonucleotide Probes

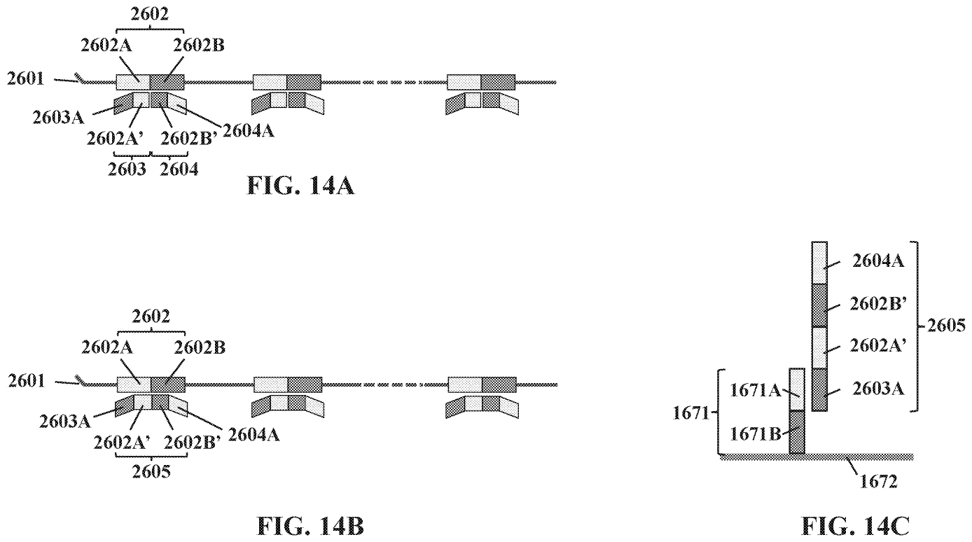

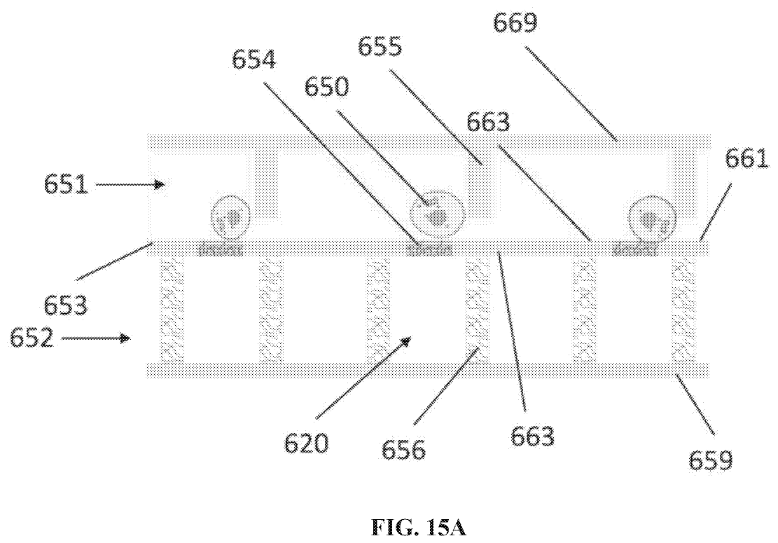

It is worth noting that certain aspects of the present application utilize unconnected oligonucleotide probes. Two or more unconnected oligonucleotide probes can couple to a sequence of interest and undergo ligation to form a ligation product that can be captured and analyzed. An example of such a method is depicted in FIGS. 14A-C. Beginning with FIG. 14A, a DNA molecule 2601 may include a sequence of interest 2602. The sequence of interest 2602 may include a first portion 2602A directly adjacent to a second portion 2602B. A first oligonucleotide probe 2603 may include a complement of the first portion 2602A′ of the sequence of interest 2602 (or a portion thereof), which may couple to the first portion 2602A. A second oligonucleotide probe 2604 may include a complement of the second portion 2602B′ of the sequence of interest 2602 (or a portion thereof), which may couple to the second portion 2602B. The first oligonucleotide probe 2603 may further include a 3′ tail 2603A that does not hybridize to the DNA molecule. The second oligonucleotide probe 2604 may further include a 5′ tail 2604A that does not hybridize to the DNA molecule. The complement of the first portion 2602A′ and the complement of the second portion 2602B′ may be hybridized directly adjacently along the sequence of interest 2602.

As depicted in FIG. 14B, the first oligonucleotide probe 2603 may then be ligated to the second oligonucleotide probe 2604 to form a ligation product 2605. Ligation may only occur when both the first oligonucleotide probe 2603 and the second oligonucleotide probe 2604 are coupled to a common sequence of interest 2602.

Next, as depicted in FIG. 14C, the ligation product 2605 may couple to a nucleic acid capture probe 1671. In this illustrated example, at least a portion of the 3′ tail 2603A of the ligation product may couple to a capture sequence 1671A of the nucleic acid capture probe 1671. The nucleic acid capture probe 1671 and/or ligation product 2605 can be extended and analyzed as described elsewhere herein. The 3′ tail and/or the 5′ tail may include unique molecular identifiers to normalize counts of the sequence of interest 2602.

### Extend Primer Using Sequence of Interest as a Template

### (i) General Overview of Method

Copy number variation and target nucleic acid location may also be determined by extending primers using each instance of the target nucleic acid (or each instance of a sequence of interest containing the target nucleic acid) as a template. The primers may be coupled to instances of a target nucleic acid of a DNA molecule and then extended using the target nucleic acid (or a portion of the target nucleic acid) and a DNA sequence flanking the target nucleic acid of the DNA molecule as a template. The primers may also be coupled to handles in sequences of interest that contain the target nucleic acid.

The primer may include a handle. The handle may facilitate primer amplification, capture, identification, or extension, and may contain a barcode sequence associated with a particular cell, fluidic device channel, or other facet of an assay. An example of such a method may include inputting a cell into a fluidic device, wherein the cell includes the DNA molecule; hybridizing a primer to a sequence of interest of the DNA molecule, wherein the sequence of interest contains a target nucleic acid sequence, and wherein the primer includes a first handle; extending the primer using: i) the target nucleic acid sequence or a portion of the sequence as a template, ii) a flanking DNA sequence as a template, wherein the flanking DNA sequence is positioned 5′ relative to the sequence of interest within the DNA molecule, iii) a second handle within the sequence as a template, or iv) a combination thereof, thereby generating an extended primer; hybridizing a random primer to the extended primer; extending the random primer using the extended primer as a template, thereby generating an extended random primer includes a complement of the handle; hybridizing the handle sequence of the extended random primer to a nucleic acid capture probe, wherein the nucleic acid capture probe is coupled to a surface of the fluidic device; extending the nucleic acid capture probe using the extended random primer as a template, thereby generating an extended nucleic acid capture probe; and sequencing the extended nucleic acid capture probe or a nucleic acid generated from the extended nucleic acid capture probe. In some cases, the method includes determining a copy number of the target nucleic acid sequence of the DNA molecule. In such implementations of the method, sequence counts of the target nucleic acid may be normalized using unique flanking DNA sequences from the extended primers or nucleic acids generated from the extended primers.

The handle may be positioned closer to the 5′ end of the sequence of interest than the target nucleic acid sequence within the sequence of interest (e.g., the relative order of components within the sequence of interest comprises, moving from a 5′ to 3′ direction, the handle followed by the target nucleic acid sequence). When this configuration is present, the primer may hybridize to the handle and then extend away from the target nucleic acid sequence, such that the extended primer does not include a complement of the target nucleic acid sequence. Alternatively, the handle may be positioned closer to the 3′ end of the sequence of interest than to the target nucleic acid sequence within the sequence of interest. When this configuration is present, the primer may hybridize to the handle and then may be extended using the target nucleic acid sequence as a template, such that the extended primer includes a complement of the target nucleic acid sequence. Sequence counts may also be normalized based on unique molecular identifiers. In some cases, the sequence of interest may include a unique molecular identifier sequence, the extending can include extending the primer using the unique molecular identifier sequence as a template. The method may also include determining a location of the target nucleic acid sequence of the DNA molecule using the flanking DNA sequence as disclosed elsewhere herein. The DNA molecule may include, for example, genomic DNA, a plasmid, a vector, viral DNA, extrachromosomal circular DNA, as well as combinations thereof. The DNA molecule may be inside of or outside of the cell during the hybridizing, extending, and/or random primer steps.

The nucleic acid capture probe can include a capture sequence that is configured to hybridize to the extended primer or extended random primer, as well as one or more additional sequences such as a spatial barcodes, a primer binding site, a unique molecular identifier, a restriction enzyme binding site, a suppressive or semi-suppressive PCR handle, a sequencing adaptor, or a combination thereof. In particular embodiments disclosed herein, the nucleic acid capture probe is coupled to a surface of the fluidic device, and the nucleic acid capture probe includes a spatial barcode that is associated with a location of the nucleic acid capture probe along the surface of the fluidic device. In particular, the nucleic acid capture probe may include a spatial barcode as disclosed elsewhere herein.

A method for analyzing a DNA molecule can include hybridizing a primer to a handle that is within or adjacent to a sequence of interest. Such a method may utilize primers that do not include 5′ handles. The method can include inputting a cell into a fluidic device, wherein the cell includes the DNA molecule; hybridizing a primer to a sequence of interest of the DNA molecule, wherein the sequence of interest includes a target nucleic acid sequence, and a handle, and wherein the primer hybridizes to the handle or at a location that is positioned 3′ relative to the handle within the sequence of interest; extending the primer using a flanking DNA sequence as a template, wherein the flanking DNA is 5′ relative to the sequence of interest of the DNA molecule, thereby generating an extended primer comprising a complement of the handle and a complement of the flanking DNA sequence; hybridizing a random primer to the extended primer; extending the random primer using the extended primer as a template, thereby generating an extended random primer comprising the handle and the flanking DNA sequence; hybridizing the handle of the extended random primer to a nucleic acid capture probe coupled to a surface of the fluidic device; extending the nucleic acid capture probe using the extended random primer as a template, thereby generating an extended nucleic acid capture probe comprising a complement of the flanking DNA sequence; and sequencing the extended nucleic acid capture probe or a nucleic acid generated from the extended nucleic acid capture probe. In some cases, the target nucleic acid includes a gene, and the gene is positioned closer to a 3′ end of the sequence of interest than the handle. In other cases, the gene is positioned closer to the 5′ end of the sequence of interest than the handle. In some cases, the gene is disposed between the handle and an additional handle. The handle (or, when present, the additional handle) may include a polyA sequence, such that the handle of the extended random primer may hybridize to a polyT sequence of the nucleic acid capture probe.

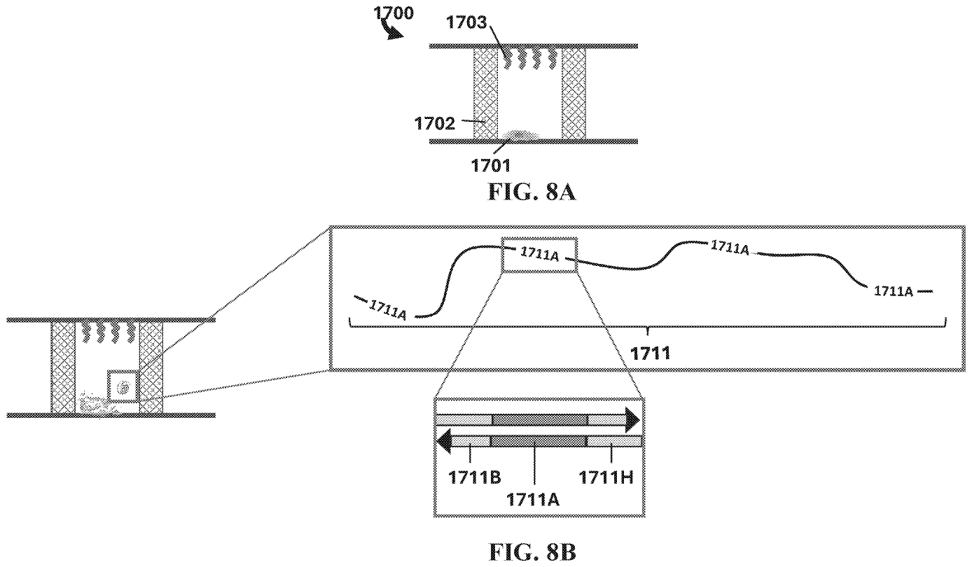

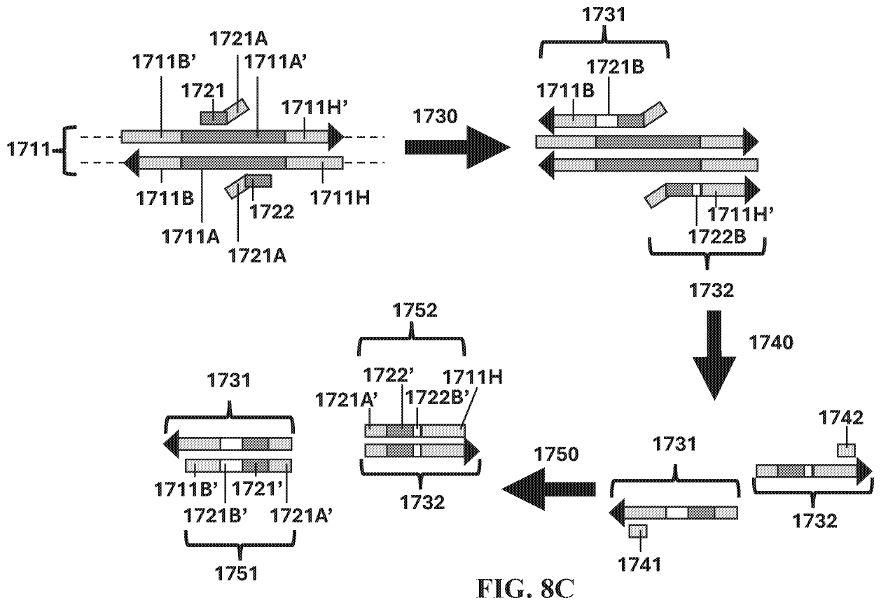

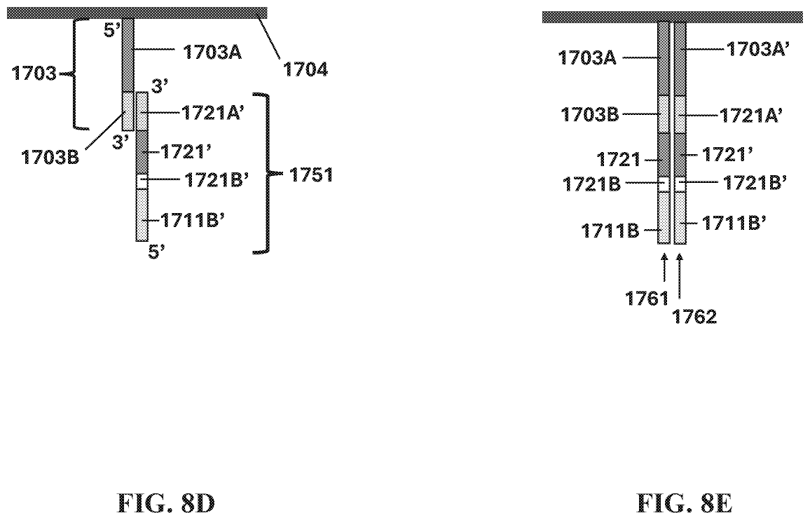

### (ii) Introduction to FIGS. 8A-E and 9A-E

Methods for using a primer to measure target nucleic acid copy number and location are depicted in FIGS. 8A-E and 9A-E. In these methods as illustrated in FIG. 8C, primers 1722, 1721 are coupled to a sequence of interest 1711A, and complement to sequence of interest 1711A′, respectively in genomic DNA 1711. Next, primers 1722, 1721 are extended in step 1730 beyond the ends of the sequences 1711A, 1711A′ to cover portions of flanking genomic DNA 1711B′, 1711H, respectively. The extended primers 1731, 1732 or random primers 1741, 1742 extended using the extended primers as templates, can then be sequenced to associate the flanking genomic DNA 1711B, 1711H sequences, and thus the genomic locations, with each instance of the sequence of interest 1711A. As each instance of the sequence of interest 1711A is likely to be adjacent to unique flanking genomic DNA 1711B, 1711H sequences, the flanking genomic DNA sequences may be used to normalize sequencing reads to determine the copy number of the target nucleic acid. While FIGS. 8A-E and 9A-E depict methods in which separate primers are coupled to forward and reverse strands within a sequence of interest 1711A, the methods may be performed with a single primer that couples to a single DNA strand.

While FIGS. 8A-E and 9A-E depict primer hybridization to DNA subsequent to cell lysis and genomic DNA 1711 release from the cell 1701, these methods may alternatively introduce primers 1721 into live cells 1701 for hybridization and extension using a sequence of interest 1711A′ as a template. In such cases, primers and extension reagents can be delivered into the cells 1701 using known transfection or transduction methods as disclosed herein. The cell 1701 may be lysed or permeabilized subsequent to extension to release extended primers to capture probes 1703 on a surface 1704 of the fluidic device 1700.

(iii) Example of Primer Method—FIGS. 8A-E

FIGS. 8A-E depict a method in which the sequence of interest 1711A is solely comprised of a gene or other sequence of interest, and the primers are directly coupled to the gene or other sequence of interest. As depicted in FIG. 8A, a cell 1701 is co-enclosed in a chamber 1702 with a plurality of nucleic acid capture probes 1703 within a fluidic device 1700. It is worth noting that the method of FIGS. 8A-E may utilize another type of compartment, such as a droplet, well, or pen. Next, as shown in FIG. 8B, the cell 1701 is lysed to release genomic DNA 1711 from the cell 1701. The genomic DNA 1711 includes multiple instances of a sequence of interest (e.g., a knock-in gene) 1711A at various positions throughout the genomic DNA 1711. In this example, each instance of the sequence of interest 1711A is positioned between first and second flanking genomic DNA 1711B, 1711H. The flanking genomic DNA 1711B, 1711H may be native genomic DNA sequences surrounding a site of insertion of the sequence of interest 1711A.

Primers 1721 are then hybridized to the multiple instances of the sequence of interest 1711A. FIG. 8C illustrates a first primer 1721 and a second primer 1722 hybridized to opposing strands of a single instance of the sequence of interest 1711A, followed by primer extension step 1730, coupling step 1740 to random primers 1741, 1742, and extension step 1750 of the random primers. The first and second primers 1721, 1722 are each coupled to capture sequences 1721A. The first and second primers 1721, 1722 may optionally include 5′ handles upstream from where the first and second primers 1721, 1722 couple to the sequence of interest 1711A, 1711A′, respectively. The first primer 1721 is extended in step 1730 using a portion of a complement of the sequence of interest 1711A′ and a portion of a complement of the first flanking DNA sequence 1711B′ as a template. The second primer 1722 is extended in step 1730 using a portion of the sequence of interest 1711A and a portion of the second flanking DNA sequence 1711H as a template. The extended first primer 1731 includes, in order, the capture sequence 1721A, the first primer 1721, a portion of the target nucleic acid sequence 1721B, and a portion of the first flanking DNA sequence 1711B. The extended second primer 1732 includes, in order, the capture sequence 1721A, the second primer 1722, an additional portion of the target nucleic acid sequence 1722B, and a complement of a portion of the second flanking DNA sequence 1711H′. The chamber may optionally be washed at a stringency (e.g. osmolarity, pH, temperature, etc.) that is sufficient to destabilize and remove non-extended primers but not so stringent as to melt extended primers from the sequence of interest 1711A. Such a wash step can remove excess primers 1721, 1722 coupled to capture probes 1703, and can thus allow an excess of primers to be input into the fluidic device 1700.

The first and second extended primers 1731, 1732 are then coupled in step 1740 to random primers 1741, 1742, and the random primers are extended in step 1750 using the first and second extended primers as templates. A first extended random primer 1751, generated by extension using the first extended primer 1731 as a template, includes, in order, a complement of the first flanking DNA sequence 1711B′, a complement of the portion of the target nucleic acid sequence 1721B′, a complement of the first primer 1721′, and a complement of the capture sequence 1721A′. A second extended random primer 1752, generated by extension using the extended second primer 1732 as a template, includes, in order, the second flanking DNA sequence 1711H, the additional portion of the target nucleic acid sequence 1722B′, a complement of the second primer 1722′, and a complement of the capture sequence 1721A′.

The extended random primers 1751, 1752 are then coupled to nucleic acid capture probes 1703. FIGS. 8D-E illustrate capture and extension of the first extended random primer 1751. However, the second extended random primer 1752 may be captured and extended in a similar fashion. As depicted in FIG. 8D, the nucleic acid capture probe 1703 can be coupled to a surface 1704 of the fluidic device 1700. The nucleic acid capture probe 1703 can include the capture sequence 1703B and one or more additional sequences 1703A such as a spatial barcode, a primer binding site, a unique molecular identifier, a restriction enzyme binding site, a suppressive or semi-suppressive PCR handle, a sequencing handle, or a combination thereof. The capture sequence 1703B of the nucleic acid capture probe 1703 can hybridize to the complement on the capture sequence 1721A′ of the first extended random primer 1751.

The nucleic acid capture probe 1703 and first extended random primer 1751 can then be extended using each other as templates. As depicted in FIG. 8E, the extended nucleic acid capture probe 1761 can include, in order, the one or more additional sequences 1703A, the capture sequence 1703B, the first primer 1721, the portion of the target nucleic acid sequence 1721B, and the first flanking DNA sequence 1711B. The further extended first random primer 1762 can include, in order, a complement of the one or more additional sequences 1703A′, a complement of the capture sequence 1721A′, a complement of the first primer 1721′, a complement of the portion of the target nucleic acid sequence 1721B′, and a complement of the first flanking DNA sequence 1711B′. Although not shown in FIG. 8E, the method may further include generating an overhang on the extended nucleic acid capture probe and further extending the extended nucleic acid capture probe using a template-switch oligonucleotide as a template as disclosed elsewhere herein. The extended nucleic acid capture probe and/or the further extended first random primer may be sequenced.

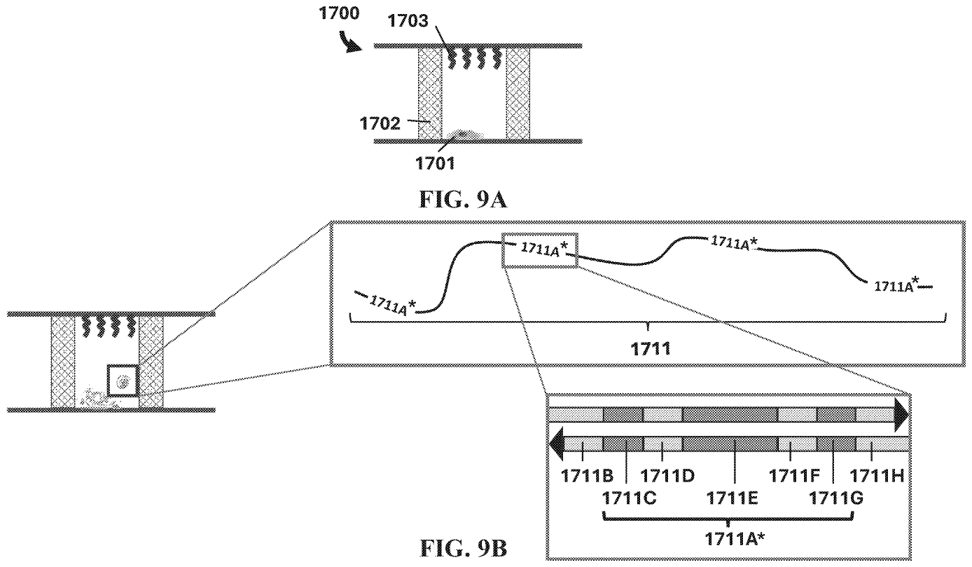

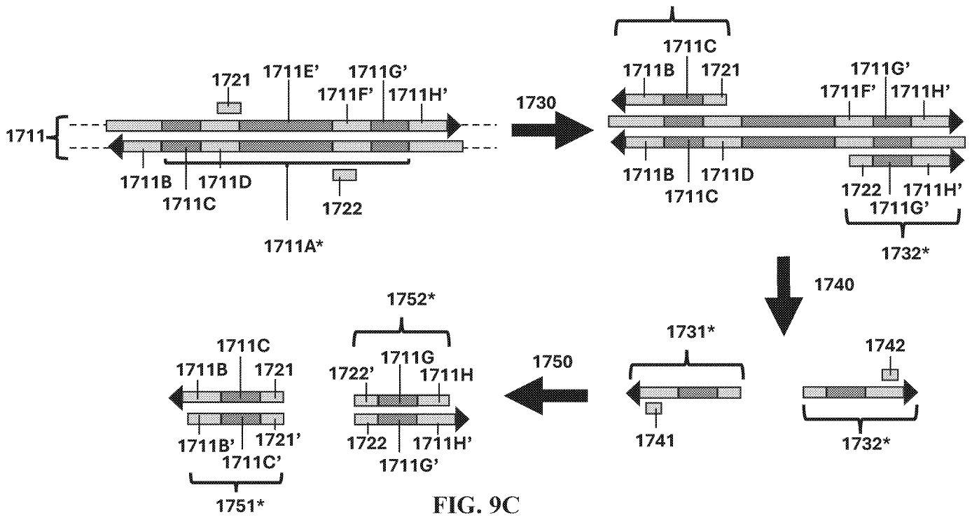

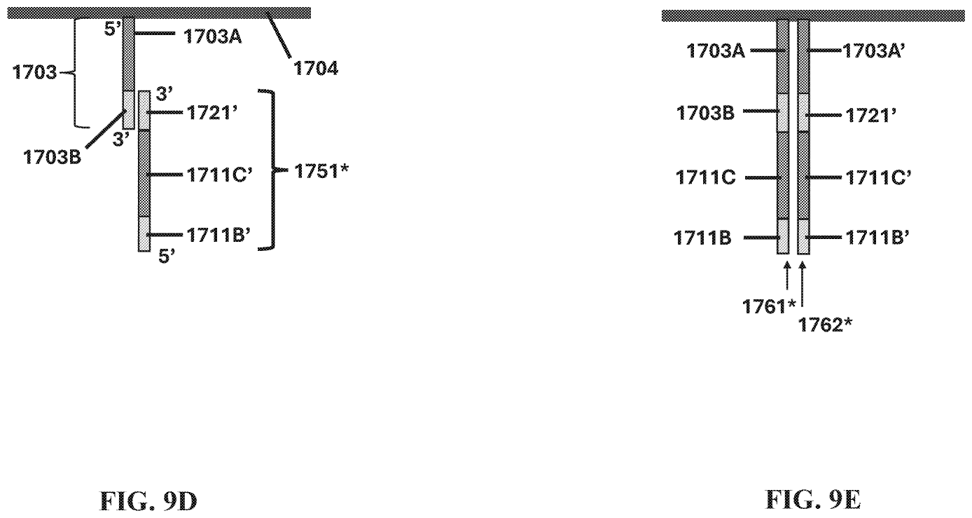

### (iv) Additional Example of Primer Method—FIGS. 9A-E

FIGS. 9A-E, illustrate an additional method for measuring the copy number and locations of a target nucleic acid sequence within a genome. In this method, the sequences of interest 1711A* are constructs that include a first handle 1711D, a second handle 1711F, and unique molecular identifiers 1711C, 1711G. Primers 1721, 1722 are hybridized to the complement of the first handle 1711D′ and the second handle 1711F, respectively, within the sequence of interest 1711A*. While the sequence of interest 1711A* in FIGS. 8A-E may, for example, be paralogs that are naturally present within a genome, the sequence of interest 1711A* in FIGS. 9A-E may be an artificial knock-in gene with engineered handles 1711D, 1711F.

As shown in FIG. 9A, in a first step, a cell 1701 is input into a fluidic device 1700 and then co-enclosed with a plurality of nucleic acid capture probes 1703 in a chamber 1702. Next, as shown in FIG. 9B, genomic DNA 1711 is released from the cell 1701 through lysis. The genomic DNA 1711 includes multiple instances of a sequence of interest 1711A. Each instance of the sequence of interest 1711A* includes, in order, a first unique molecular identifier 1711C, a first handle 1711D, a target nucleic acid 1711E (e.g., a gene disposed within the sequence of interest 1711A*), a second handle 1711F, and a second unique molecular identifier 1711G. The sequence of interest 1711A* is disposed between a first flanking DNA sequence 1711B and a second flanking DNA sequence 1711H.

As shown in FIG. 9C, primers 1721, 1722 may be hybridized to the complement of the first handle 1711D′ and the second handle 1711F. The primers may then be extended in step 1730 and coupled in step 1740 to random primers, and the random primers 1741, 1742 can be extended in step 1750. Specifically, a first primer 1721 may hybridize to a complement of the first handle 1711D′, while a second primer 1722 may hybridize to the second handle 1711F. Following extension step 1730, the extended first primer 1731* may include, in order, the first primer 1721, the first unique molecular identifier 1711C, and the first flanking DNA sequence 1711B. The extended second primer 1732* may include, in order, the second primer 1722, a complement of the second unique molecular identifier 1711G′, and a complement of the second flanking DNA sequence 1711H′. The extended first and second primers 1731*, 1732* are then coupled in step 1740 to random primers 1741, 1742, respectively, and the random primers are extended in step 1750 using the extended first and second primers as templates. A first extended random primer 1751*, generated by extension using the extended first primer 1731* as a template, includes, in order, a complement of the first flanking DNA sequence 1711B′, a complement of the first unique molecular identifier 1711C′, and a complement of the first primer 1721′. A second extended random primer 1752*, generated by extension using the extended second primer 1732* as a template, includes, in order, the second flanking DNA sequence 1711H, the second unique molecular identifier 1711G, and a complement of the second primer 1722′.

The extended random primers 1751*, 1752* can then be coupled to nucleic acid capture probes 1703. FIG. 9D illustrates the first extended random primer 1751 coupled to a nucleic acid capture probe 1703. The nucleic acid capture probe includes a capture sequence 1703B that is configured to couple to portions of the first and second extended random primers 1751*, 1752*, and one or more additional sequences 1703A. Although the second extended random primer 1752* is not shown in FIG. 9D or 9E, the methods depicted in FIGS. 9D and 9E may be performed on the second extended random primer 1752* as described for the first extended random primer 1751*. Whereas FIG. 9D shows the complement of the first primer 1721′ of the first extended random primer coupled to the capture sequence 1703B of the nucleic acid capture probe 1703, the complement of the second primer 1722′ of the second extended random primer 1752* can also couple to the capture sequence 1703B of the nucleic acid capture probe 1703.

Returning to FIG. 9D, the capture probe contains a capture sequence 1703B that hybridizes to the complement of the first primer 1721′ of the first random primer 1751*. As an example of such a motif, the complement of the primer 1721′ can include a poly A sequence and the capture sequence 1703B on the nucleic acid capture probes 1703 can include polyT sequences. The nucleic acid capture probes 1703 can also include one or more additional sequences 1703A as described elsewhere herein, and may be coupled to a surface 1704 of the fluidic device 1700.

The extended first random primer 1751* and nucleic acid capture probe 1703 can then be extended, and the resulting extended nucleic acid capture probe 1761* can optionally be further extended using a template switch oligonucleotide as disclosed elsewhere herein. As illustrated in FIG. 9E, the extended nucleic acid capture probe 1761*, generated by extension using the extended first random primer 1751* as a template, includes, in order, the one or more additional sequences 1703A, the capture sequence 1703B, the unique molecular identifier 1711C, and the first flanking DNA sequence 1711B. The further extended first random primer 1762*, now further extended using the nucleic acid capture probe 1703 as a template, includes, in order, a complement of the one or more additional sequences 1703A′, a complement of the first primer 1721′, a complement of the first unique molecular identifier 1711C′, and a complement of the first flanking DNA sequence 1711B′. The extended nucleic acid capture probe and/or further extended random primer may then be sequenced. The number of instances of the sequence of interest 1711A in the genomic DNA 1711 can then be calculated as the number of sequence reads with unique flanking genomic DNA complements (1711B′), unique molecular identifiers, or a combination thereof. The location of each instance of the target nucleic acid sequence can be determined by referencing the flanking genomic DNA complements 1711B′ against a full genomic sequence of the organism from which the genomic DNA 1711 was obtained. Capture and extension may be performed on the extended second random primer 1752* in an analogous manner as shown and described for the extended first random primer 1751* in FIGS. 9D-E.

### 5′ Sequencing

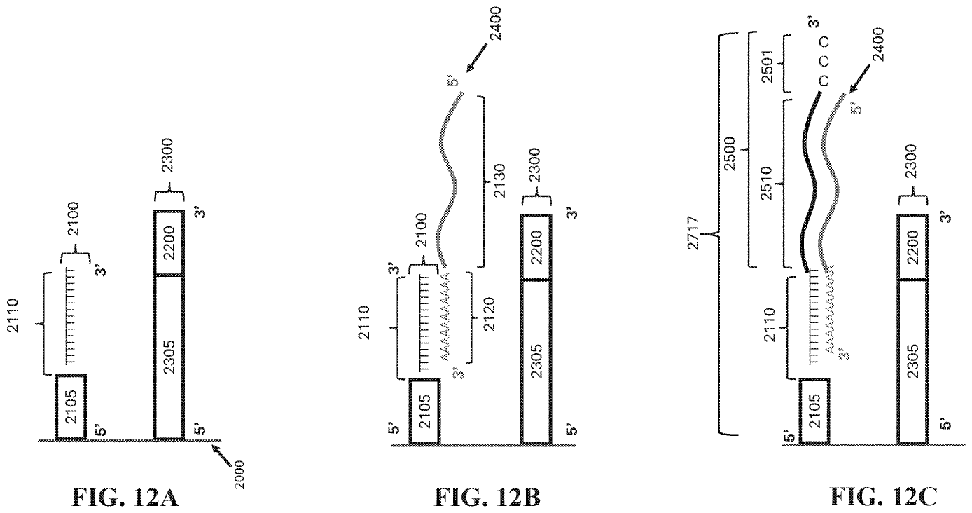

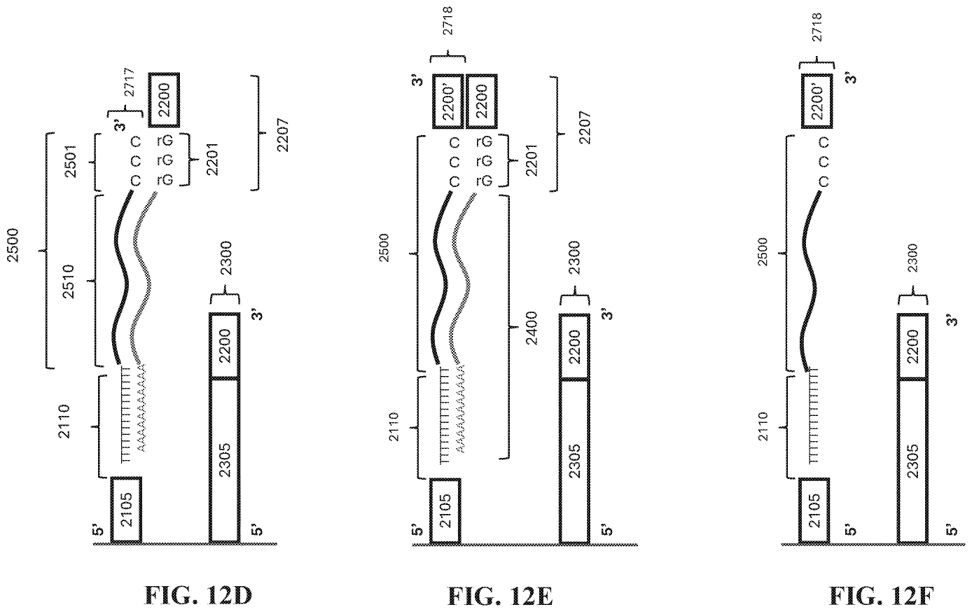

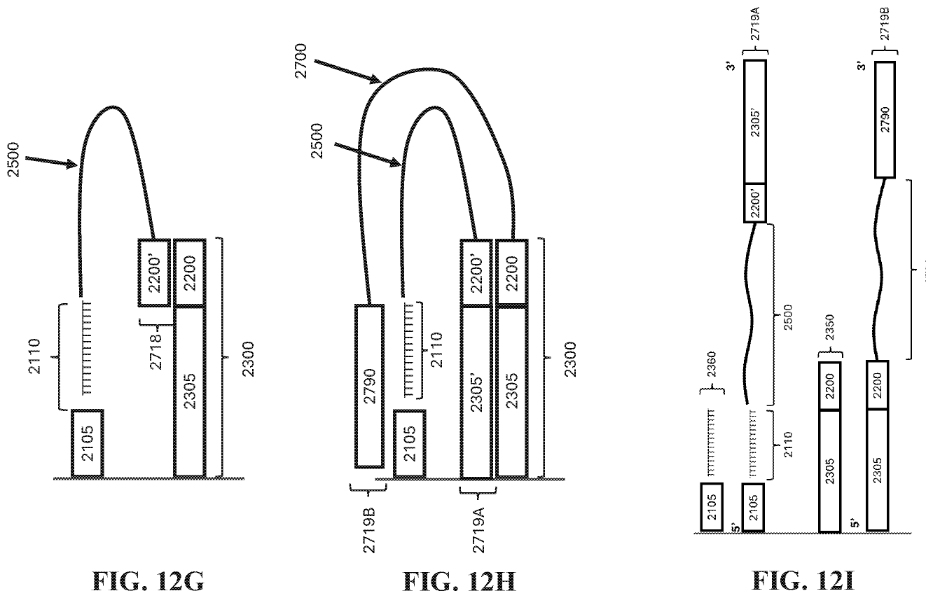

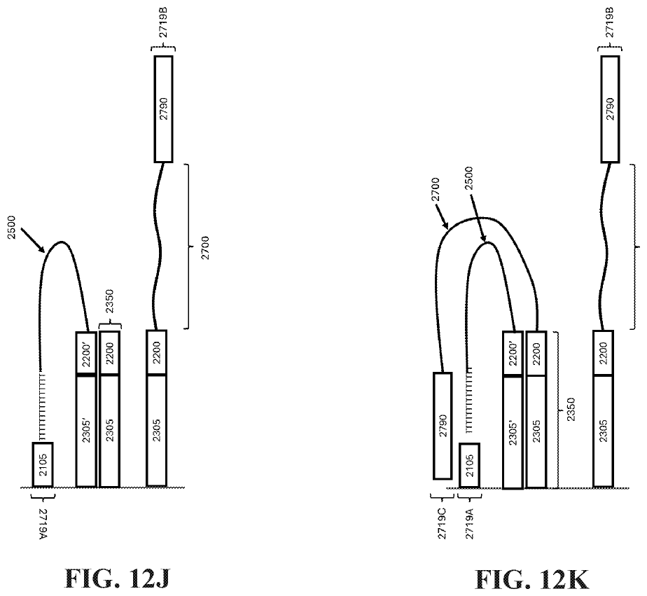

Further disclosed herein are methods for incorporating sequences onto the 3′ ends of extended nucleic acid capture probes and onto the 5′ ends of target nucleic acids and sequences of interest. FIGS. 12A-K cover an example of these methods. While FIGS. 12A-K are described in detail elsewhere herein, the reference numbers from these figures are included in the foregoing paragraph for guidance. The nucleic acid capture probes 2100 (e.g., see FIG. 12A) disclosed herein may be coupled to a surface 2000 of a substrate or fluidic device by their 5′ ends. Under this orientation, 3′ ends of the nucleic acid capture probes 2100 may undergo extension using a target nucleic acid 2400 (e.g., nucleic acid fragments, extended primers, linearized circularization probes, or amplicons; see FIG. 12B) as a template. The extended nucleic acid capture probes 2717 may then include, from 5′ to 3′, the nucleic acid capture probe followed by an extension sequence 2500 (i.e., a complement of the target nucleic acid or a complement of a portion of the target nucleic acid; see FIG. 12B). Certain next-generation sequencing methods are read length limited, and are therefore incapable of reading portions of the sequences from the nucleic acid capture probe at the 3′ end of an extension sequence 2500 (corresponding to a 5′ end of the target nucleic acid 2400). When the nucleic acid capture probes include barcode sequences (barcode sequences may be present within 2105 in FIG. 12A; examples of barcode sequences include spatial barcode sequences), the barcode sequences may be absent from sequencing reads that cover the 5′ end of the target nucleic acid 2400.

To address this limitation, and to extend the present methods to 5′ target nucleic acid sequence analysis on short read next-generation sequencing platforms, the present disclosure provides barcode primers 2300 (e.g., see FIG. 12A) for 5′ barcoding. Following nucleic acid capture probe 2100 extension, 3′ ends of extended nucleic acid capture probes (e.g., see 3′ end 2200′ of further extended nucleic acid capture probe 2718 in FIGS. 12F and 12G) can hybridize to barcode primers 2300 on the same substrate or surface. The barcode primers 2300 can include additional barcode sequences 2305. The extended nucleic acid capture probes 2718 can be further extended using the barcode primers 2300 as templates, encoding complements of the additional barcode sequences 2305 from the barcode primers 2300 onto the further extended nucleic acid capture probes 2718. The barcode primers 2300 may also optionally extend using the further extended nucleic acid capture probes 2718 as templates. The resultant extended nucleic acids (e.g., see 2719A and 2719B in FIG. 12I) can thus include barcodes at either end or at both ends to enable sequencing from the 5′ or 3′ end of a target nucleic acid.

Sequences may be added to 3′ ends of extended nucleic acid capture probes 2717 to facilitate hybridization to barcode primers. The barcode primers 2300 present in a fluidic device may share common terminal capture sequences 2200. The sequences 2200′ added to the 3′ extensions 2500 on extended nucleic acid capture probes 2717 may include complements of the terminal capture sequences 2200 of the barcode primers 2300. As an example, the 3′ extensions 2500 on extended nucleic acid capture probes 2717 may be further extended using template-switch oligonucleotides 2207 as templates, wherein the template-switch oligonucleotides include copies of the terminal capture sequences 2200 of the barcode primers 2300, thereby adding complements of the barcode primer 2300 terminal capture sequences 2200 to the 3′ ends of the further extended nucleic acid capture probes 2718.

FIGS. 12A-K illustrate a method for generating a 3′ barcode on an extended nucleic acid capture probe (corresponding to the 5′ end of the target nucleic acid that served as a template for nucleic acid capture probe extension). As shown in FIG. 12A, a surface can include a nucleic acid capture probe 2100 and a barcode primer 2300 disposed thereon. The nucleic acid capture probe 2100 can include one or more additional sequences 2105, such as a primer binding site, a barcode, a unique molecular identifier, a sequencing adapter, or a combination thereof. Similar to the nucleic acid capture probe 2100, the barcode primer 2300 can include a capture sequence 2200, and may optionally further include one or more additional sequences 2305, such as a primer binding site, a barcode, a unique molecular identifier, a sequencing adaptor, or a combination thereof.

A target nucleic acid molecule such as a nucleic acid fragment, an extended primer, a linearized circularization probe, or an amplicon can couple to the nucleic acid capture probe 2100. FIG. 12B illustrates a target nucleic acid molecule 2400 with a poly(A) tail 2120 hybridized to a poly(T) tail 2110 of the nucleic acid capture probe 2100. The target nucleic acid molecule also includes a sequence of interest 2130 such as a knock-in gene, a barcode, or a combination thereof.

As shown in FIG. 12C, the nucleic acid capture probe 2100 can then be extended (e.g., reverse transcribed) using the sequence of interest 2130 as a template, producing a 3′ extension 2500 coupled to the 3′ end of the nucleic acid capture probe 2100. The 3′ extension 2500 can include a sequence 2510, wherein the sequence 2510 is complementary to the target nucleic acid molecule 2130. The 3′ extension 2500 may be formed by extending and growing at the 3′ end of the extending molecule and terminating in an overhang 2501, such as an oligocytosine sequence generated during reverse transcription.

The 3′ extension 2500 can then be further extended to include a complement of the barcode primer capture sequence 2200. The one or more additional sequences 2105, poly(T) tail 2110, a sequence 2510 (complementary to the target nucleic acid molecule 2130), and overhang 2501 in aggregate can be referred to as an extended nucleic acid capture probe 2717. For example, as depicted in FIG. 12D, an oligoguanosine motif 2201 of a template-switch oligonucleotide (TSO) 2207 can be hybridized to the overhang 2501. The 3′ extension 2500 can be further extended using the template-switch oligonucleotide 2207 as a template. The TSO 2207 may include a copy of the barcode primer capture sequence 2200 or a portion of the barcode primer capture sequence 2200. An example of a product generated by this further extension is illustrated in FIG. 12E, which shows the further extended nucleic acid capture probe 2718 that includes the 3′ extension 2500 and a complement of the barcode primer capture sequence 2200′. FIG. 12F illustrates the further extended nucleic acid capture probe 2718 with the 3′ extension 2500 and the complement of the barcode primer capture sequence 2200′ after the captured target nucleic acid molecule 2400 and TSO 2207 have been decoupled from the extended nucleic acid capture probe 2718, in accordance with some embodiments.

Moving to FIG. 12G, the complement of the barcode primer capture sequence 2200′ on the further extended nucleic acid capture probe 2718 can hybridize to the barcode primer capture sequence 2200. In a subsequent concerted step, the further extended nucleic acid capture probe 2718 can be extended using the barcode primer 2300 as a template and the barcode primer 2300 can be extended using the further extended nucleic acid capture probe 2718 as a template. As illustrated in FIG. 12H, following this extension step, the yet further extended nucleic acid capture probe 2719A can include, in order, the one or more additional sequences 2105, the poly(T) sequence 2110, the 3′ extension 2500, the complement of the barcode primer capture sequence 2200′, and a complement of the one or more additional sequences of the barcode primer 2305′. The barcode primer 2300 can also be extended using the further extended nucleic acid capture probe 2718 as a template. The extended barcode primer 2719B can include, in order, the one or more additional sequence 2305, the capture sequence 2200, a sequence 2700 that is complementary to 3′ extension 2500 of the further extended nucleic acid capture probe 2718, and a sequence 2790 that is complementary to the nucleic acid capture probe 2100 (shown as 2105 and 2110 in FIG. 12H).

FIG. 12I illustrates the yet further extended nucleic acid capture probe 2719A, the extended barcode primer 2719B, an additional nucleic acid capture probe 2360, and an additional barcode primer 2350 on the surface. The additional nucleic acid capture probe 2360 and additional barcode primer 2350 can be identical to the nucleic acid capture probe 2100 and the barcode primer 2300, respectively.

The yet further extended nucleic acid capture probe 2719A and the extended barcoded primer 2719B can participate in turnaround steps by respectively serving as extension templates for additional barcode primers 2350 or additional nucleic acid capture probes 2360. As shown in FIG. 12J, the complement of the barcode primer capture sequence 2200′ of the yet further extended nucleic acid capture probe 2719A can hybridize to the capture sequence 2200 of the additional barcode primer 2350. This hybridization step or a melting and rehybridization step (FIG. 12J) followed by subsequent extension (FIG. 12K) may be referred to as a “turnaround.” Although not shown in FIGS. 12J-K, during a turnaround step, an additional nucleic acid capture probe 2360 may hybridize to and then extend using an extended barcode primer 2719B as a template. A method may utilize any number of turnaround steps, including about 1 to 5, about 1 to 10, about 1 to 15, about 1 to 20, about 1 to 25, about 1 to 30, about 1 to 50, about 5 to 10, about 5 to 15, about 5 to 20, about 5 to 25, about 5 to 30, about 5 to 50, about 10 to 15, about 10 to 20, about 10 to 25, about 10 to 30, about 10 to 50, about 15 to 20, about 15 to 25, about 15 to 30, about 15 to 50, about 20 to 25, about 20 to 30, about 20 to 50, or about 25 to 50.

As illustrated in FIG. 12K, the additional barcode primer 2350 can be extended using the yet further extended nucleic acid capture probe 2719A as a template, thereby generating an additional extended barcode primer 2719C that is identical to the extended barcode primer 2719B.

### Compartments

The term “compartment,” as used herein, generally, refers to a space or volume that may be suitable to contain one or more species or conduct one or more reactions. The compartment may isolate a space or volume from another space or volume. The compartment may comprise one or more other (inner) compartments. In some aspects, it should be noted that the space or volume of a compartment can be an open space or open volume that is occupied by air or liquid. A compartment can have an open space or open volume that is defined by a wall. In various examples, the open space or open volume is not filled with a polymeric material. The wall can be made of a polymer that has an outer surface and inner surface where the inner surface forms at least a part of the boundary of the open space or open volume and the outer surface forms at least a part of the boundary to the area outside of the compartment.

In some aspects, a compartment includes a chamber. A method disclosed herein can include synthesizing one or more chambers that at least partially enclose the one or more cells in a fluidic device. As detailed further herein, the one or more cells may be subjected to one or more forms of analysis while they are enclosed in the one or more chambers. The one or more chambers may co-enclose the one or more cells with nucleic acid capture probes that are configured to capture one or more cellular nucleic acids from a cell.

In many embodiments disclosed herein, the one or more chambers are comprised of one or more polymer matrices formed with the fluidic space. As used herein, the term “polymer matrix” generally refers to a phase material (e.g. continuous phase material) that comprises at least one polymer. In some embodiments, the polymer matrix refers to the at least one polymer as well as the open interstitial space within the polymer. A polymer matrix may comprise a polymerized form of a polymer precursor, which generally refers to one or more molecules that upon activation can trigger or initiate a polymeric reaction. A polymer precursor can be activated by electrochemical energy, photochemical energy, a photon (i.e., light), magnetic energy, or any other suitable energy. Energy can include thermal energy, radiant energy, chemical energy, plasma energy, mechanical energy, elastic energy, or acoustic energy, for example. As used herein, the term “polymer precursor” includes monomers (e.g., that are polymerized to produce a polymer matrix) and/or crosslinking compounds, which may include photo-initiators, other compounds necessary or useful for generating polymer matrices (e.g., porogen).

In particular embodiments, the one or more chambers comprise a hydrogel. As used herein, the term “hydrogel” can refer to a polymeric material that is not water soluble or is poorly water soluble but can contain water (e.g., at least 10% by weight) when hydrated.

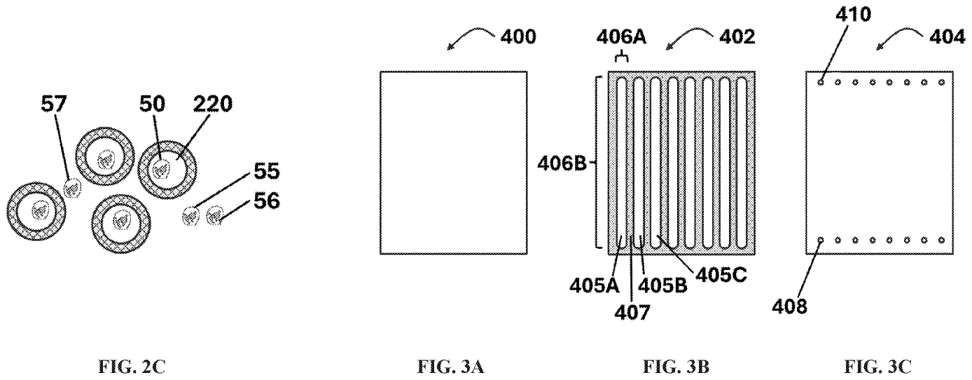

FIGS. 2A-C illustrate an example of a method for forming a chamber enclosing one or more biological components in a fluidic device disclosed herein. FIG. 2A shows a portion of a system as provided herein (e.g., comprising a fluidic device as disclosed herein) including an energy source forming a polymer matrix around biological components 50 and 51. FIG. 2B shows a polymer matrix being formed around another biological component 52 in a portion of a system as provided herein. FIG. 2C is a top view schematic showing top view of a channel with a polymer matrix formed around a biological component in a system as provided herein. As illustrated in FIGS. 2A-2C, in some embodiments, the one or more chambers have cylindrical shapes that extend from the bottom layer to the top layer of a fluidic device.

With continued reference to FIG. 2A, the channel 200 of the system may include a first surface 201 provided by the bottom layer and a second surface 202 provided by the top layer of the fluidic device. The energy source 203 may comprise one or more energy emitting portions (e.g., an energy emitting portion 205). In some embodiments, the energy source 203 may comprise one or more non-emitting portions (e.g., a non-emitting portion 204). The non-emitting portion 204 does not emit energy for forming a polymer structure, but can be configured at a later time to emit energy. In some embodiments, the emitting portion 205 can emit energy in the form of electromagnetic waves (e.g., microwaves, light, heat, etc.) to at least a portion of the fluidic device. For example, the energy source may comprise an LED array in which individual LEDs can be selectively activated (e.g., act as an energy emitting portion 205) to create light projections with specified patterns.

In further reference to FIG. 2A, the polymer matrix 208, 209, or at least a portion of the polymer matrix 208, 209, may be coupled to the first surface 201, the second surface 202, or both surfaces 201, 202. The polymer matrix 208, 209 may enclose a space 220 that contains a biological component 50. In various embodiments, the polymer matrix 208, 209 may extend from the first surface 201 to the second surface 202 (e.g., through at least a portion of a lumen of the channel 200) such that the polymer matrix surrounds, or substantially surrounds, the biological component 50. FIG. 2B shows polymer matrices 218, 219 formed surrounding the biological component 52. Biological component 53 was separated from biological component 52, and is not present in FIG. 2B. In some embodiments, separation of the biological components is achieved through fluidic pressure, flow pulsation, dielectrophoresis, optothermal flow, or some combination thereof. FIG. 2C provides a top-down view of the channel 200 depicted in FIG. 2B, wherein the cylindrical shapes of the polymer matrix define a compartment 220 surrounding the biological component 50. Additional biological components 55, 56, 57, which are not enclosed within polymer matrices, are shown in FIG. 2C.

In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid crystal based. In some embodiments, the fluidic channel may be coupled to or disposed on a movable stage. In other embodiments, light may be projected to or onto at least a portion of the first fluidic channel to generate one or more polymer matrices. The light may be directed to various parts of the first fluidic channel. The energy source (e.g., light source) may be coupled to the fluidic device via an objective (e.g., a microscope objective or lens). The energy source may be directed to a portion of the fluidic channel (e.g., via a movable objective). In some cases, the light source, the objective, and/or the fluidic channel are movable to allow emission of energy to the fluidic channel so as to generate a pattern on at least a portion of a surface of the fluidic device. The polymer matrix may be formed similarly or complementary to the pattern of energy emission.

In some embodiments, such chambers have annular-like cross-sections. As used herein, the term “annular-like cross-section” means a cross section topologically equivalent to an annulus. In some embodiments, the inner space, or interior, of a chamber has an inner diameter from 5 μm to 500 μm and a volume in the range of from about 1 nanoliter to 100 nanoliters, or from about 1 nanoliter to 10 nanoliters. In some embodiments, the polymer matrix wall has a thickness from about 1 μm (micrometer) to about 30 μm or about 5 to about 15 μm. In some embodiments, a chamber extends from a bottom surface to a top surface of a fluidic device. In some embodiments, a chamber has a height of about 10 μm to about 500 μm. In some embodiments, a polymer matrix wall having an annular-like cross-section has an aspect ratio (i.e., height/width) of 1 or less. In some embodiments, aspect ratio and polymer matrix wall thickness are selected to maximize chamber stability against forces, such as reagent flow through the channel, washings, and the like.

In some embodiments, the generation of a polymer matrix within said fluidic device comprises exposing the one or more polymer precursors to an energy source. In some embodiments, the energy source is a light generating device. In some embodiments, the light generating device generates light ranging from about 350 nm to about 800 nm. In some embodiments, the generation of the polymer matrix comprises from about 1 to about 50 seconds of illumination or from about 2 to about 4 seconds of illumination.

Optionally, a first chamber of the one or more chambers can be disposed inside of a second chamber of the one or more chambers. This design can be utilized to separately partition two species (e.g., a cell and a reagent or two cells) within close proximity. This design can also be used to control the timing with which two species are contacted. For example, a method can include forming a first chamber around a cell, flowing a bead (or other assay reagent incapable of diffusing into the first chamber) adjacent to the cell, forming a second chamber surrounding the first chamber and enclosing the bead, and selectively degrading the first chamber to allow the cell and bead to come into contact within the second chamber.

In some embodiments, a functional group can be coupled to one or more chambers. Some non-limiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized chamber may be used to capture a biomolecule enclosed therein, thereby trapping the biomolecule in proximity to a biological component (e.g., a cell) enclosed within the chamber. The biomolecule may be produced by the biological component (e.g., secretome from a cell). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

A fluidic device disclosed herein can include a detector that is configured to detect one or more locations of one or more biological components contained within a channel. Returning to FIGS. 2A-B, in certain embodiments, the energy source 203 can comprise, be coupled to, or be in communication with a detector that detects, or is configured to detect, a location of a biological component in the fluidic device. In some embodiments, the image may be obtained from a camera (e.g., a digital camera, fluorescent imaging camera, etc.). In some embodiments, the camera may be coupled to, connected to, or in communication with the energy source 203. For example, the camera (not shown) may be in electrical communication with the energy source 203. In some embodiments, the energy source 203 may comprise the camera. In various embodiments, the energy source 203 may comprise a microscope (e.g., a fluorescence microscope, a confocal microscope, lens-free imaging system, a transmission electron microscopy (TEM), a scanning electron microscope (SEM), etc.). The microscope may be used to detect one or more positions of one or more biological components (e.g., in combination with the detector).

In some embodiments, one or more chambers has sufficiently large pores to allow movement or transfer of a reagent (e.g., an enzyme, a chemical compound, a small molecule, an antibody, etc.) therethrough, but sufficiently small so as to retain biological components (e.g., cells). Alternatively or additionally (e.g., when two or more chambers are synthesized with different polymer precursors or radiation powers), one or more chambers can have sufficiently small pores to prevent movement or transfer of a reagent and/or biological component (e.g., DNA, RNA, a protein, a cell, etc.) through the walls of the chamber. In some embodiments, the pores have a diameter from 5 nm to 100 nm. In some embodiments, the pores have a diameter smaller than 5 nm. Pore size may be selected based on the sizes of reagents and analytes in an assay. For example, pore size may be modulated by adjusting polymer precursor formulation, porogen size, pH, salt concentration, photopolymerization light duration and light intensity, and the like. In some embodiments, the average diameter of pores of a chamber prevent passage of molecules having a molecular weight of 25 kiloDaltons (kDa) or greater; or having a molecular weight of 50 kDa or greater; or having a molecular weight of 75 kDa or greater; or having a molecular weight of 100 kDa or greater; or having a molecular weight of 150 kDa or greater. In some embodiments, the pores of a chamber are sufficiently large for 150 kDa species such as antibodies, as well as smaller reagents, to flow through, but prohibitively small for cells or beads to flow through. In some embodiments, DNA or RNA retained have lengths that are sequenceable using conventional sequencing-by-synthesis techniques. For example, such DNA or RNA comprise at least 50 nucleotides, or in some embodiments, at least 100 nucleotides. In some embodiments, the pores may have an average diameter from 5 nm to 100 nm.

In some cases, the analysis channel may include polymer matrix structures for capturing or trapping a biological component or a molecule or compound produced by the biological component (e.g., prior to introduction of the biological components into the fluidic device). For example, a user may obtain an analysis channel that includes polymer matrix structures. That is, the user may not form the polymer matrix structures. In various cases, the analysis channel may be configured to include polymer matrix structures for capturing or trapping a biological component or a molecule or compound produced by the biological component. For example, in such embodiments, subsequent to introduction of the biological components into the fluidic device and the analysis channel, one or more polymer matrix structures may be formed in the analysis channel. The analysis channel may be configured for a screening process, a library preparation, or another suitable process. In some embodiments, the screening process may be for drug screening, antibiotic screening, culture conditions screening, or CRISPR screening. In certain cases, a plurality of samples may be placed into a plurality of channels. The plurality of samples may be screened against a variety of conditions in other signal-containing channels.

The sealable aperture may be configured to transition from a sealed state to an open state. For example, a sealable aperture may comprise a heat sensitive polymer that can melt, for example, upon receiving heat and render the sealable aperture open. In some cases, the passage of the biological component through the sealable aperture may be inhibited in the sealed state. In certain cases, the passage of the biological component through the sealable aperture may be allowed in the open state. In some cases, the sealable aperture may be sealed with an agarose gel, a temperature-soluble polymer, an N-isopropylacrylamide (NIPAAm) polymer, a wax compound, an alginate, or any other suitable compound or material.

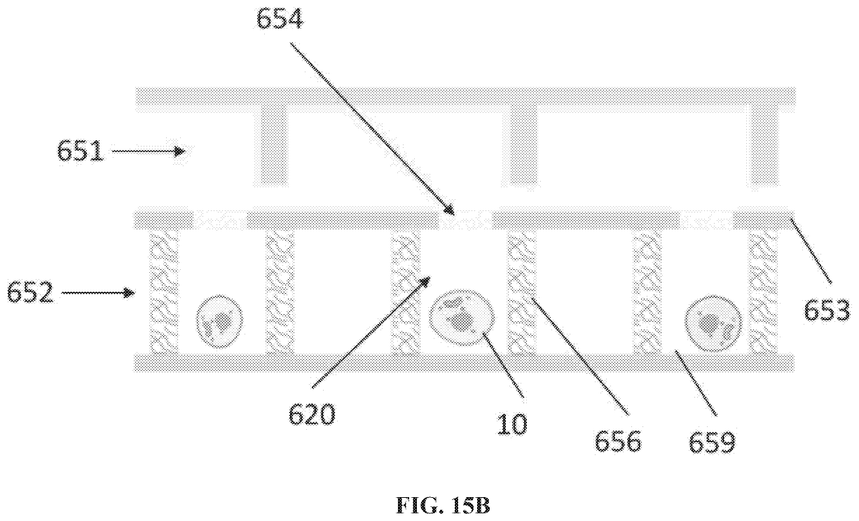

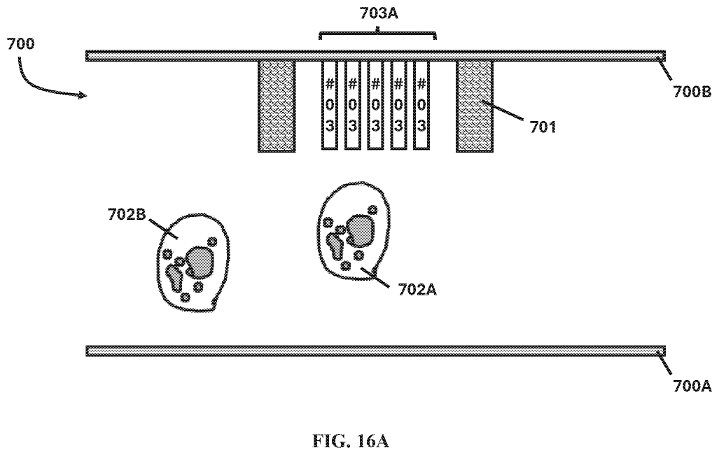

FIGS. 15A and 15B show a portion of a fluidic device configured to trap a biological component 50. The fluidic device may comprise a flow channel or chamber 651, an analysis channel or chamber 652, and a layer or wall 653 disposed between at least a portion of the flow channel 651 and the analysis channel 652. The layer 653 may comprise one or more sealable apertures or openings 654. Additionally, one or more flow inhabitation elements 655 may inhibit or prevent, or be configured to inhibit or prevent, the biological component 50 from flowing along the flow channel 611. A flow inhibition element 655 may be configured to stop or trap the biological component 50 adjacent to a sealable aperture 654. As described herein, the sealable aperture 614 may be configured to transition from a sealed state (e.g., a closed state) or configuration to an open state or configuration. FIG. 15A shows an example of the sealable aperture 614 in a sealed state. FIG. 15B shows an example of the sealable aperture 654 in an open state. Upon transitioning to a sealed state to an open state, the sealable aperture 654 may allow or permit passage of the biological component 50 from at least a portion of the flow channel 651 to at least a portion of the analysis channel 652. In certain instances, the analysis channel 652 may be placed, or configured to be placed, below the flow channel 651 to allow the biological component 50 to be transferred to the analysis channel 652 from the flow channel 651 by a force provided (e.g., via gravity, high pressure pulse by pressurizing a flow in the flow channel, and generating negative pressure in the analysis channel). In some embodiments, the fluidic device may be spun or centrifuged to disposed the one or more biological components from the flow channel to the analysis channel. Reagents can be disposed or passed through at least a portion of the analysis channel 652, for example, to conduct analyses or experiments are provided herein.

As shown in FIG. 15A, the flow inhibition element 655 may be disposed within at least a portion of the flow channel 651 to inhibit or prevent flow of a biological component (e.g., biological component 50) in the flow channel 651. The flow inhibition element 655 may be configured to capture or trap the biological component 50 in at least a portion of the flow channel 651. In some cases, the flow inhibition element 655 may extend from a surface (e.g., surface 669) of the flow channel 651. In some cases, the surface 669 may be disposed opposite of a flow channel surface 661, which is adjacent to the layer 653.

In various cases, the analysis channel 652 may comprise a surface 659 disposed opposite of the analysis channel surface 663, which is adjacent to or a surface of the layer 653. The analysis channel 652 may comprise one or more polymer matrices 656. The analysis channel 652 may comprise one or more polymer precursors. For example, one or more polymer precursors may be disposed in at least a portion of the analysis channel 652. The one or more polymer matrices 656 may be formed using an energy source which provides energy to the one or more polymer precursors in the analysis channel 602. The energy source may be in optical communication, electrochemical communication, electromagnetic communication, thermal communication, or microwave communication with the fluidic device or the analysis channel 652. In some cases, the energy source may be a light generating device, a heat generating device, an electrochemical generating device, an electrode, a microwave device, or a combination thereof. The energy source may selectively provide energy to the analysis channel 652 to form polymer matrices at predefined locations. A spatial energy modulating element may be used to selectively provide energy to the analysis channel 652.

In some cases, the spatial energy modulating element may comprise a photolithographic mask, a DMD system, or other suitable mask. The one or more polymer matrices 656 may be formed before the sealable aperture 654 transitions to an open state (e.g., as shown in FIG. 15A). For example, a polymer matrix may be formed and aligned with the sealable aperture such that the biological component 650 held by the inhibition element 655 may directed (e.g., fall by gravity or by fluid pressure) into a compartment 620 when the scalable aperture 654 is rendered open. The one or more polymer matrices 656 may be formed after the sealable aperture 654 transitions to an open state (e.g., as shown in FIG. 15B). The one or more polymer matrices 656 may form an analysis chamber or compartment 620, as described herein.

In some aspects, a compartment may be formed by expanding a stimulus-responsive polymer to enclose a biological material. The stimulus-responsive polymer may expand from a contracted state to an expanded state in response to a stimulus such as temperature, osmolarity, pH, or a combination of stimuli thereof. In the contracted state, the stimulus-responsive polymer may form a portion of a compartment that includes an opening through which a biological material such as a cell may flow through. After a biological material flows into the opening, the stimulus responsive polymer may be expanded, which may shrink or close the opening to form a compartment with the biological material trapped therein. The stimulus may be applied in a spatially controlled manner, for example by using light directed from a spatial light modulator to selectively heat (e.g., in the presence of thermoresponsive particles) individual stimulus-responsive polymers within a fluidic device. The stimulus may also be applied throughout a fluidic device or a portion of a fluidic device, for example by heating or replacing a buffer in a fluidic device or an individual fluidic channel within a fluidic device. Examples of stimulus-responsive polymers consistent with the present disclosure include poly(N-isopropylacrylamide) (PNIPAM), poly(N-vinylcaprolactam) (PVCL), poly(acrylic acid) (PAA), poly(methacrylic acid) (PMAA), poly(itaconic acid) (PIA), poly(glutamic acid) (PGA), poly(aspartic acid) (PASP), a poly(β-amino ester) (PBAE), poly(L-histidine) (PLH), poly(2-vinylpyridine) (P2VP), poly(4-vinylpyridine) (P4VP), poly(dimethylaminoethyl methacrylate) (PDMAEMA), poly(diethylaminoethyl methacrylate) (PDEAEMA), poly(allylamine hydrochloride) (PAH), poly(styrene sulfonate) (PSS), poly(acrylamide-co-acrylic acid) (PAM-co-PAA), poly(acrylamide-co-dimethylaminoethyl methacrylate) (PAM-co-PDMAEMA), poly(acrylamide-co-diethylaminoethyl methacrylate) (PAM-co-PDEAEMA), as well as combinations thereof.

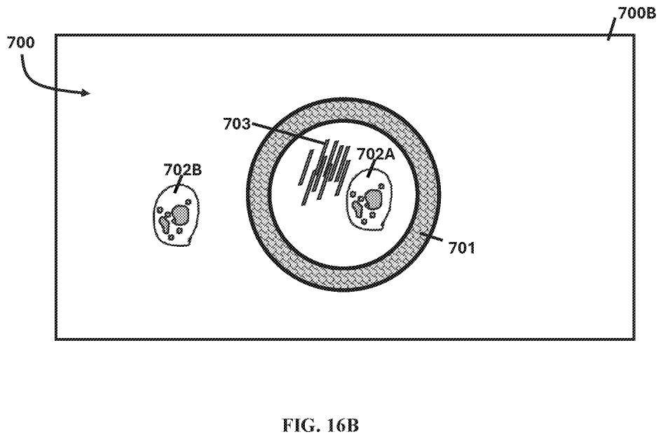

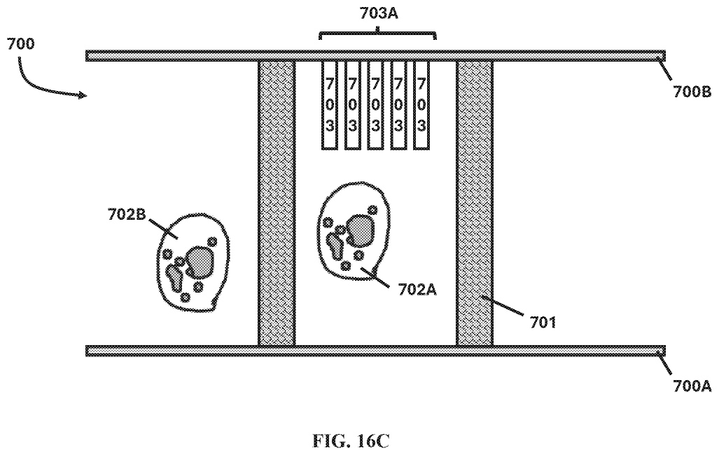

An example of a method for forming a compartment using a stimulus-responsive polymer is illustrated in FIGS. 16A-C. FIG. 16A provides a cross-sectional side-on view of a portion of a fluidic device (700). The fluidic device (700) may include a bottom surface (700A) and a top surface (700B) that opposes (e.g., is substantially parallel to) the bottom surface (700A). In this example, the top surface (700B) includes a discrete site (703A) with nucleic acid capture probes as described elsewhere herein. In this figure, the stimulus-responsive polymer (701) is in a contracted state, and only extends partway from the top surface (700B) along the height of the fluidic device (700). Such a structure may be fabricated, for example, by forming the stimulus-responsive polymer (701) on the top surface prior to fluidic device (700) fabrication.

With continued reference to FIG. 16A, biological materials (702A, 702B) may be input into the fluidic device (700). In this example, a first biological material (702A) may diffuse into a region that is circumscribed or otherwise surrounded by the stimulus-responsive polymer

(701). A second biological material (702B) remains outside of the area occupied or surrounded by the stimulus-responsive polymer (701). For example, the stimulus-responsive polymer can be formed by a controlled photopolymerization or 3D printing or by a physical printing process to transfer the stimulus-responsive polymer to the top surface (700B).

FIG. 16B provides a bottom view of the portion of the fluidic device (700) depicted in FIG. 16A. From this view, the shape of the stimulus-responsive polymer (701) coupled to the top surface (700B) is visible. The first biological material (702A) is inside of a region surrounded by the stimulus-responsive polymer (701), while the second biological material (702B) is outside of this region.

Next, as shown in FIG. 16C, which is an illustration of the cross-sectional side-on view of the fluidic device (700) as shown in FIG. 16A, a stimulus may be applied to the stimulus-responsive polymer (701), causing the stimulus-responsive polymer (701) to transition from its contracted state to its expanded state. The expanded stimulus-responsive polymer (701) extends from the top surface (700B) to the bottom surface (700A), thereby, along with the top surface (700A) and the bottom surface (700B), defining a compartment that encloses the first biological material (702A). The second biological material (702B) is not enclosed in the compartment, and may optionally be washed out of the fluidic device (700) while the first biological material (700A) is trapped within the compartment. It should be noted that the stimulus-responsive polymer (701), the top surface (700A) and the bottom surface (700B) cooperate to form an open interior volume that contains the first biological material (702A) and discrete site (703A)

In some cases, the compartments are droplets. A droplet can serve as a reaction chamber for one or more cells. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. In some cases, the droplets are encapsulated in oil. In some cases, the droplets comprise one or more beads. In some cases, each droplet comprises a single bead. A bead can be a gel bead. In some cases, a bead comprises one or more oligonucleotides. The oligonucleotides can be used to capture one or more components from a cell in the droplet. The oligonucleotides can include a barcode unique to the bead which they are disposed on. Within each droplet, a cell can be lysed, and nucleic acids from the cell (including messenger RNA) can bind to a unique barcode on the gel bead, which can then be used to identify the cell of origin. In some cases, the droplets are formed by mixing cells and barcoded gel beads with oil to create the droplets. Enclosing a nucleic acid molecule or a derivative thereof or a cell comprising the nucleic acid molecule or a derivative thereof and one or more reagents may comprise flowing a first phase comprising an aqueous fluid, the cell, and the one or more reagents and a second phase comprising a fluid that is immiscible with the aqueous fluid toward a junction. Upon interaction of the first and second phases, a discrete droplet of the first phase comprising the nucleic acid molecule or a derivative thereof or a cell comprising the nucleic acid molecule or a derivative thereof and the one or more reagents may be formed. In some cases, the compartment may comprise a single cell. The cell may be lysed or permeabilized within the compartment (e.g., droplet) to provide access to the nucleic acid molecule of the cell. In some cases, reagents (including reverse transcription reagents) are also included during droplet formation. One or more reagents may be co-enclosed with a nucleic acid molecule or a derivative thereof or a cell comprising the nucleic acid molecule or a derivative thereof. For example, a nucleic acid molecule or a derivative thereof or a cell comprising the nucleic acid molecule or a derivative thereof may be co-enclosed with one or more reagents selected from the group consisting of lysis agents or buffers, permeabilizing agents, enzymes (e.g., enzymes capable of digesting one or more RNA molecules, extending one or more nucleic acid molecules, reverse transcribing an RNA molecule, permeabilizing or lysing a cell, or carrying out other actions), fluorophores, oligonucleotides, primers, probes, barcodes, nucleic acid barcode molecules (e.g., nucleic acid barcode molecules comprising one or more barcode sequences), buffers, deoxynucleotide triphosphates, detergents, reducing agents, chelating agents, oxidizing agents, nanoparticles, beads, and antibodies. In some cases, a nucleic acid molecule or a derivative thereof, or a cell comprising the nucleic acid molecule or a derivative thereof, may be co-enclosed with one or more reagents selected from the group consisting of temperature-sensitive enzymes, pH-sensitive enzymes, light-sensitive enzymes, reverse transcriptases, proteases, ligase, polymerases, restriction enzymes, nucleases, protease inhibitors, exonucleases, and nuclease inhibitors. Droplets may have overall volumes that are less than about 5000 pL, 2500 pL, 1000 pL, 500 pL, 250 pL, 100 pL, 50 pL, or less.

As described herein, one or more processes may be performed in a compartment, which may be a well. The well may be a well of a plurality of wells of a substrate, such as a microwell of a microwell array or plate, or the well may be a microwell or microchamber of a device (e.g., microfluidic device) comprising a substrate. The well may also be a nanowell. The well may be a well of a well array or plate, or the well may be a well or chamber of a device (e.g., fluidic device). Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid. The well may have a volume of less than 1 milliliter (mL). For instance, the well may be configured to hold a volume of at most 1000 microliters (pL), at most 100 pL, at most 10 pL, at most 1 pL, at most 100 nanoliters (nL), at most 10 nL, at most 1 nL, at most 100 picoliters (pL), at most 10 (pL), or less. The well may be configured to hold a volume of about 1000 pL, about 100 pL, about 10 pL, about 1 pL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 pL, at least 10 pL, at least 100 pL, at least 1000 pL, or more. The well may be configured to hold a volume in a range of volumes listed herein, for example, from about 5 nL to about 20 nL, from about 1 nL to about 100 nL, from about 500 pL to about 100 pL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well. In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

In some cases, cells can be enclosed using optofluidic technology. A fluidic device can include one or more distinct regions, which can be at least partially physically separated from each other. In some cases, these distinct regions may be referred to as “pens.” For example, a fluidic device can include a channel in fluidic communication with a plurality of pens. A pen may be open on a side of the pen that abuts or is adjacent to a channel. Cells can be loaded into the channel. Energy, including light, can be applied to the fluidic device. The projected light can change electrical properties of certain regions of the fluidic device to create electrical fields that push or pull cells. This light energy can be used to pick up single cells or a certain number of cells, move the single cell or cells, and place the single cell or cells in a specific pen within the fluidic device. This can result in a pen of a fluidic device comprising a single cell. Once isolated, cells may remain in the pen and kept alive with controlled media flow. The pens within a fluidic device can be continuously monitored. As described elsewhere herein, any type of assay may be performed on a single cell or multiple cells within a pen (functional assays, imaging assays, morphology assays, proteomic assays, metabolic assays, etc.). Nucleic acids can be released from the cells and contained within the pen.

In some cases, cells are spatially constrained to a unique area of the fluidic device, with or without a physical compartment. In some cases, each of the unique areas is no more than 285,000 μm², 200,000 μm², 150,000 μm², 100,000 μm², 50,000 μm², 25,000 μm², 20,000 μm², 15,000 μm², 12,000 μm², 11,000 μm², 5,000 μm², 1,000 μm², 500 μm², 100 μm², 10 μm², or 1 μm². In some cases, a physical compartment as described elsewhere herein is used to constrain the area a cell can move. In some cases, a cell is constrained to an area without the use of a physical compartment. The present disclosure also provides systems including one or more capture elements for immobilizing and/or compartmentalizing one or more biological components (including cells). The system can include a fluidic device. The fluidic device can include or contain one or more biological components. In some embodiments, the fluidic device can include a first surface (e.g., in a channel and/or chamber of the fluidic device). The fluidic device can include one or more capture elements. The capture elements can immobilize, or be configured to immobilize, at least one of the one or more biological components at a location on or adjacent to the first surface (or any suitable surface). Immobilization or coupling of a biological component to a capture element can form an immobilized biological component. In some embodiments, the one or more capture elements comprise a physical trap (including a divot), a geometric trap, a well, an electrochemical trap, a chemical affinity trap, one or more magnetic particles, an electrophoretic trap, a dielectrophoretic trap, or a combination thereof. In some embodiments, the chemical affinity trap comprises streptavidin, an antibody, or a combination thereof. In some embodiments, the electrochemical trap comprises a gold electrode, a platinum electrode, an indium tin oxide (ITO) electrode, or other suitable electrochemical trap. In some embodiments, the one or more capture elements are disposed in a pattern on the first surface. In some embodiments, the one or more capture elements comprises a well as described elsewhere herein. For example, a cell can be constrained to a specific area of a fluidic device by using a capture element. A biological component (including a cell) can couple to a capture element on a surface, for example. Once coupled to the capture element, the biological component may be constrained to a specific area of the fluidic device. As described elsewhere herein, any number and combination of assays can be performed on a spatially constrained biological component.

### Fluidic Device

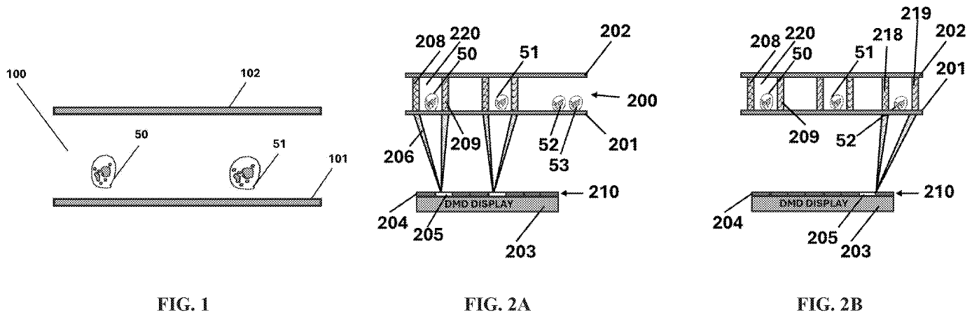

FIG. 1 illustrates cells disposed within a channel 100 of a fluidic device. A channel 100 of a fluidic device can also sometimes be referred to as a “flow chamber,” “flow channel,” or “reaction chamber”), and may receive or be configured to receive a biological sample. FIG. 1 shows a simplified schematic cross-sectional side view illustration of a portion of a channel 100 of a fluidic device. The channel 100 may comprise a first surface 101 and a second surface 102 positioned opposite of one another (e.g., as depicted in FIG. 1). In some embodiments, a middle spacer layer of double-sided adhesive with a cut-out portion can be used to position the first surface 101 and second surface 102 in a facing relationship to at least partly form the flow channel 100. The perpendicular distance between the first surface 101 and the second surface depends in part on the nature and size of the biological components to be analyzed. In some embodiments, such as, those adapted to analyzing mammalian cells, the perpendicular distance between a first surface and a second surface may be in the range of from 10 μm to 500 μm, or in the range of from 50 μm to 250 μm. In some embodiments, the perpendicular distance between a first surface and a second surface may be in the range of from twice the average size of the biological component to be analyzed to five times the average size of the biological component to be analyzed. In some embodiments, the first surface 101 may be a lower surface. In certain embodiments, the second surface 102 may be an upper surface. In some embodiments, the first surface 101 and second surface 102 are substantially parallel, so that the perpendicular distance between them is substantially the same throughout the channel, for example, where chambers are formed. The perpendicular distance may vary by less than +/−10%, 5%, 1%, or 0.1% throughout the channel 100. The channel 100 may receive a biological sample comprising one or more biological components 50, 51. The channel 100 may receive one or more polymer precursors. As illustrated in FIG. 1, the biological components 50, 51 may include cells. However, as discussed herein, the biological components may include tissues, proteins, nucleic acids, etc. In some embodiments, the first surface 101 and/or second surface 102 can be optically transmissive so that visible and UV light can transmit through one or both of the surface for the generation of polymeric hydrogels, imaging of the flow cell, and the measurement of the analyte and biological components.

In certain cases, a channel may have a cross-sectional area that is rectangular, circular, semi-circular, or oval. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

In certain cases, a channel may have a cross-sectional area that is rectangular, circular, semi-circular, or oval. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

The first surface 101, the second surface 102, or both surfaces 101, 102 may be functionalized, for example with a coating. As a non-limiting example, a surface coating may be a surface polymer. Some non-limiting examples of surface coatings may include a capture reagent (e.g., pyridinecarboxaldehyde (PCA)), a functional group to capture one or more moieties (e.g., a chemical moiety), an acrylamide, an agarose, a biotin, a streptavidin, a strep-tag II, a linker, a functional group comprising an aldehyde, a phosphate, a silicate, an ester, an acid, an amide, an alkyne, an azide, an aldehyde dithiolane, or a combination thereof. In various embodiments, the surface coating may include a functional group to capture one or more moieties. For example, the acrylamide, the agarose, etc. may include such a functional group. In certain embodiments, the surface polymer may comprise polyethylene glycol (PEG), a thiol, an alkene, an alkyne, an azide, or combinations thereof. In various embodiments, the surface polymer may comprise a silane polymer derivatized with N-hydroxysuccinimide. In some embodiments, the surface polymer may be functionalized with at least one of an oligonucleotide, an antibody, a cytokine, a chemokine, a protein, an antibody derivative, an antibody fragment, a carbohydrate, a toxin, or an aptamer. In particular embodiments, the surface coating comprises a material for which adherent cells have a binding affinity, such as fibronectin, laminin, poly-L-ornithine, or combinations thereof.

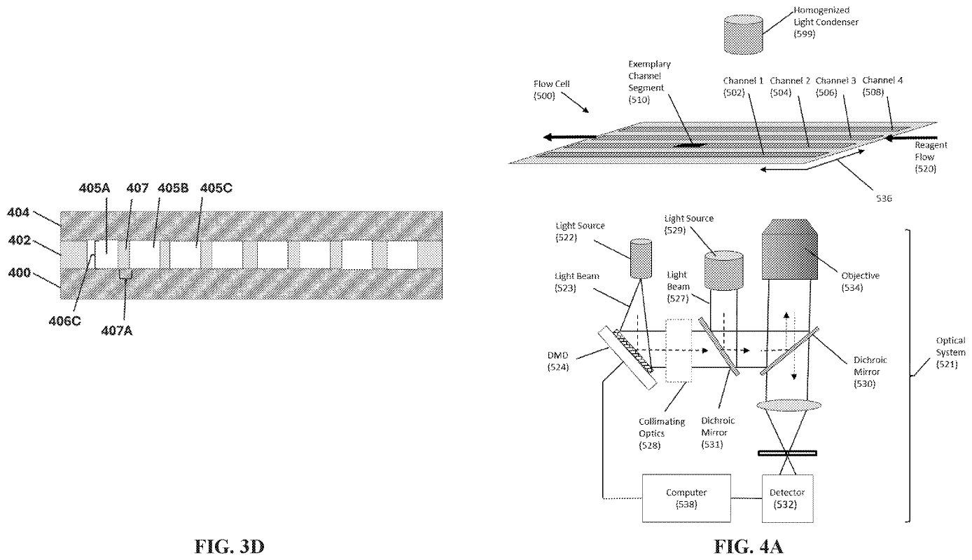

FIGS. 3A-D illustrate an example of a fluidic device consistent with the present disclosure. The channel 100 of FIG. 1 may correspond to any one of the cut-out regions (405A, 405B, 405C) of FIG. 3D. FIGS. 3A-D figures depict the components of a flow cell comprised of a bottom layer 400 (FIG. 3A), a spacer layer 402 (FIG. 3B), and top layer 404 (FIG. 3C). The bottom layer (400) and the top layer (404) can both independently be a glass or plastic material. In some cases, the top layer (404) is optically transparent or translucent. In some cases, the bottom layer (400) is optically transparent or translucent. In many cases, the top layer (404) and the bottom layer (400) are optically transparent or translucent. The spacer layer (402) can be a double-sided pressure sensitive adhesive with one or more cut-out regions (405A, 405B, 405C). In this design, the one or more cut-out regions of the spacer layer are sandwiched between the bottom layer and the top layer to form one or more channels. In various embodiments, the spacer layer (402) includes a core plastic (e.g., PET) layer with pressure sensitive adhesive coating on its top and/or bottom sides that contact the top (404) and bottom (400) layers. Examples of adhesive coatings consistent with the present disclosure include siloxane, silicone, acrylate, acrylamide, polyvinyl, polyurethane, epoxy, polyphenol, polyester, polyamide, polyimide, polytetrafluoroethylene, polyethylene, polypropylene, polycarbamate, polycarbonate, polyacrylic acid, sulfonated polyester, and combinations thereof.

Referring to FIG. 3B, a peripheral portion (407) of the spacer layer (402) provides a boundary for one or more cut-out regions (405A, 405B, 405C) with defined widths (406A) and lengths (406B). In many designs disclosed herein, the spacer layer (402) adheres to the top (404) and bottom (400) layers, such that a cut-out region or plurality of cut-out regions (405A, 405B, 405C) define a channel or a plurality of channels. The spacer layer (402), bottom layer (400), and/or top layer (404) can be water (or optionally more generally liquid) impermeable such that a first aqueous sample in a first cut-out region (e.g., 405A) is isolated from a second aqueous sample in a second cut-out region (e.g., 405B). Biological components and reagents in a first cut-out region (e.g., 405A) may similarly be isolated from other cut-regions (405B, 405C). The one or more cut-out regions (405A, 405B, 405C) can thus define flow cell channels with defined dimensions.

A channel defined by a cut-out region (405A, 405B, 405C) can be operably coupled to an inlet (408) and/or an outlet (410) in the top layer (404), through which, for example, a gas, liquid, sample, or reagent may flow. In various embodiments, the inlet (408) and outlet (410) can both be represented as through holes in the top layer (404). However, the inlet (408) and/or outlet (410) can be disposed within other components of the flow cell, such as the bottom layer (400) or spacer layer (402).

With respect to FIG. 3D, the top layer (404) can have a height of about 0.7 mm and the bottom layer can have a height of about 0.5 mm. The first cut-out region (406) can have a height (406C) defined by the distance between opposing faces of the top layer (404) and the bottom layer (400). The spacer layer can have a height ranging from about 50 microns to about 200 microns, and preferably, from about 70 microns to about 130 microns. The cut-out region can have dimensions of about 10.3 cm length x about 0.7 cm width.

### Polymer Precursors

A channel of a fluidic device (e.g., the channel) can comprise one or more polymer precursors for forming chambers. The polymer precursor may be light activatable. For example, synthesizing one or more chambers can comprise projecting light into the channel with a spatial energy modulating element such that the projected light causes cross-linking of one or more polymer precursors to at least partially form the one or more chambers. As discussed in further detail below, a wide variety of photo-synthesizable gels and degradable gels are available for implementing the systems and methods described herein. In some embodiments, photo-synthesized gels are formed using a photo-initiator for radical polymerization. In some embodiments, photo-initiators comprise 2-hydroxy-4′-(2-hydroxyethoxy)-2-methylpropiophenone (Irgacure 2959), lithium phenyl-2,4,6-trimethylbenzoylphosphinate (LAP), or Eosin-Y. In some embodiments, one or more polymer precursors comprise hyaluronic acid, chitosan, heparin, alginate, polyethylene glycol (PEG), multi-arm PEG, poly(ethylene glycol)-b-poly(propylene oxide)-b-poly(ethylene glycol) (PEG-PPO-PEG), poly(lactic acid-co-glycolic acid)-b-poly(ethylene glycol)-b-poly(lactic acid-co-glycolic acid) (PLGA-PEG-PLGA), and poly(vinyl alcohol). In some embodiments, polymer precursors comprise PEG or multi-arm PEG. In some embodiments, the polymer precursors comprise an enzymatically degradable cross-linker. In some embodiments, such enzymatically degradable cross-linker is degradable by an esterase or a peptidase. In some embodiments, polymer precursors comprise a photo-degradable cross-linker. In some embodiments, such photo-degradable cross-linker comprises a nitrobenzyl group. In some embodiments, such photo-degradable cross-linker comprises a coumarin moiety.

In some embodiments, the one or more polymer precursors are added to the fluidic device along with the one or more cells. In various embodiments, the one or more cells and the one or more polymer precursors can be pre-mixed and then added to the fluidic device at the same time; the one or more cells and the one or more polymer precursors can be added to the fluidic device at the same time; the one or more cells can be added to the fluidic device first and then the one or more polymer precursors can be added to the fluidic device second; the one or more cells can be added to the fluidic device first allowing the one or more cells couple to a surface in the fluidic device, and then the one or more polymer precursors can be added to the fluidic device second; or the one or more polymer precursors can be added to the fluidic device first and then the one or more cells can be added to the fluidic device second.

Polymer precursors may be selected from a wide variety of compounds including, but not limited to, polyethylene glycol (PEG)-thiol, PEG-acrylate, acrylamide, N,N′-bis(acryloyl) cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or combinations or mixtures thereof. Representative crosslinkers useful for polymer synthesis are listed in Tables 1A and 1B. Degradation agents that may be used to degrade chambers formed from particular polymer precursors are indicated in column 4 of Table 1A (column 4).

TABLE 1A

| | | | Degradation |
|---|---|---|---|
| Precursors | Hydrogels | Crosslinkers | Agents |
| Acrylamide | Poly- | Bis-acryloyl cystamine | DTT/TCEP/THP |
| | acrylamide | (Structure 1) | |
| PEG-based | PEG | Bis(2- | DTT/TCEP/THP |
| acryloyl | | methacryloly)oxyethyl | |
| | | disulfide (Structure 2) | |
| Dextran- | Dextran | N,N′-(1,2- | NaIO₄ |
| based | | Dihydroxylethylene)bis- | |
| acryloyl | | acrylamide (Structure 3) | |
| Poly- | Poly- | Structure 4 | NaOH, |
| sacchride- | saccharide | | ethanolamine, |
| base | | | DTT/TCEP/THP |
| acryloyl | | | |
| Gelatin-base | Gelatin | Structure 5 | NaOH, |
| acryloyl | | | ethanolamine, |
| | | | nucleophilic bases |
| — | — | Structure 6 | NaOH, alkali, |
| | | | organic bases |
| — | — | Structure 7 | Acid |

TABLE 1B

| Structure | |
|---|---|
| Number | Formula |
| 1 | 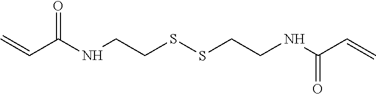 [SMILES: C=CC(=O)NCCSSCCNC(=O)C=C] |
| 2 | 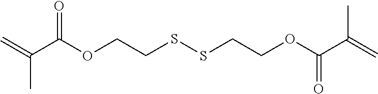 [SMILES: C=C(C)C(=O)OCCSSCCOC(=O)C(=C)C] |
| 3 | 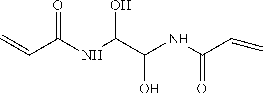 |
| 4 | 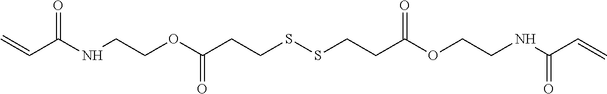 [SMILES: C=CC(=O)NCCOC(=O)CCSSCCC(=O)OCCNC(=O)C=C] |
| 5 | 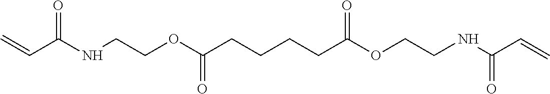 [SMILES: C=CC(=O)NCCOC(=O)CCCCC(=O)OCCNC(=O)C=C] |
| 6 | 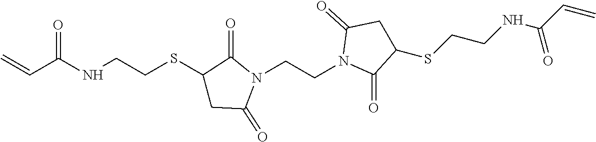 [SMILES: C=CC(=O)NCCSC1CC(=O)N(CCN2C(=O)CC(SCCNC(=O)C=C)C2=O)C1=O] |
| 7 | 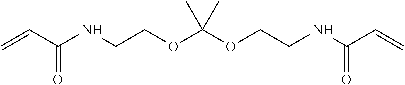 [SMILES: C=CC(=O)NCCOC(C)(C)OCCNC(=O)C=C] |
| 8 | 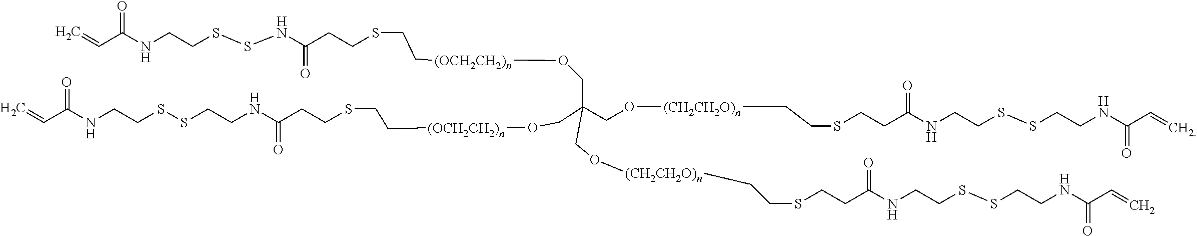 |
| | In some cases, each instance of n is independently an integer selected from about 1 |
| | to about 500. |
| 9 | 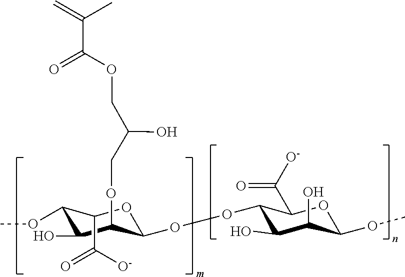 |
| | In some cases, n and m are independently integers selected from about 1 to about |
| | 500, wherein at least one of n and m is at least 1. In some cases, n and m are each |
| | at least 1. |
| 10 | 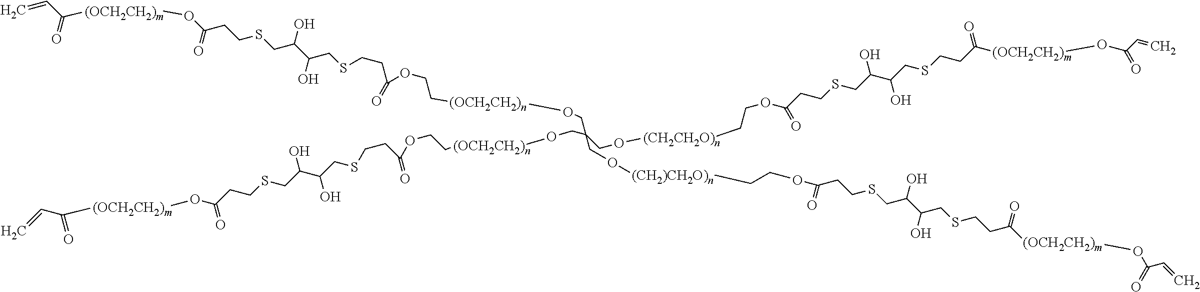 |
| | In some cases, each instance of n is independently an integer selected from about 1 |
| | to about 500. In some cases, each instance of m is independently an integer |
| | selected from about 1 to about 500. |
| 11 | 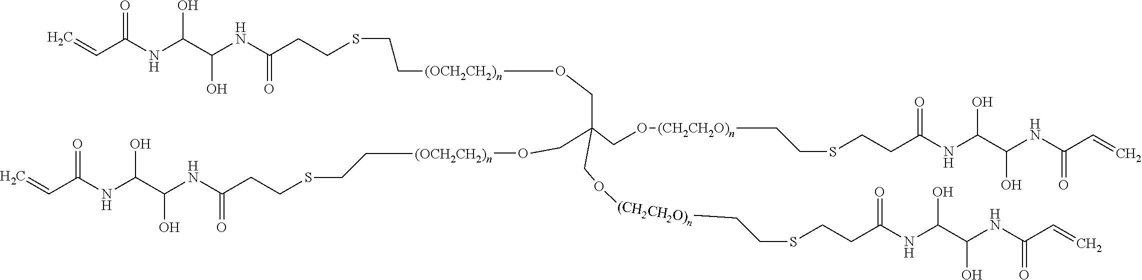 |
| | In some cases, each instance of n is independently an integer selected from about 1 |
| | to about 500. |
| 12 | 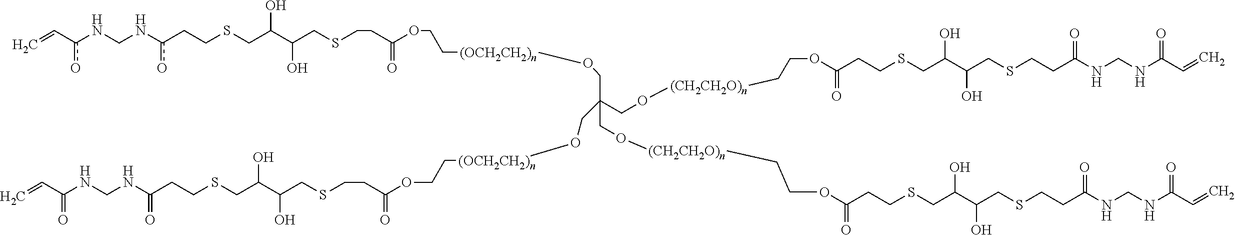 |
| | In some cases, each instance of n is independently an integer selected from about 0 |
| | to about 500. |
| 13 | 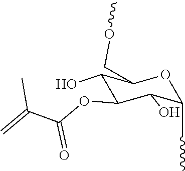 |
| | In some cases, structure 13 comprises from about 1 to about 500 monosaccharide |
| | units. |
| 14 | 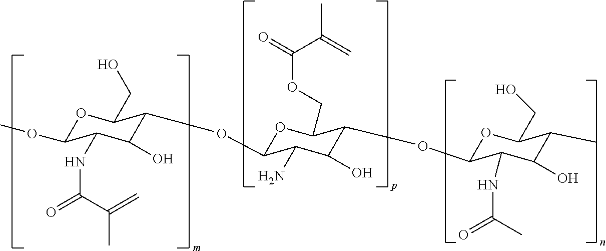 |
| | In some cases, n, m, and p are independently integers selected from about 1 to |
| | about 500, wherein at least one of n, m, and p is at least 1. In some cases, each of |
| | n, m, and p are at least 1. |
| 15 | 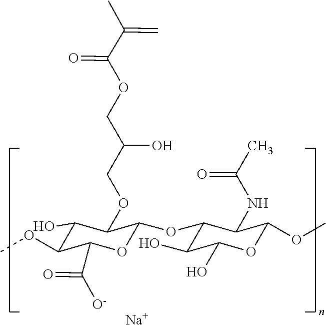 |
| | In some cases, n is an integer selected from about 1 to about 500. |
| 16 | 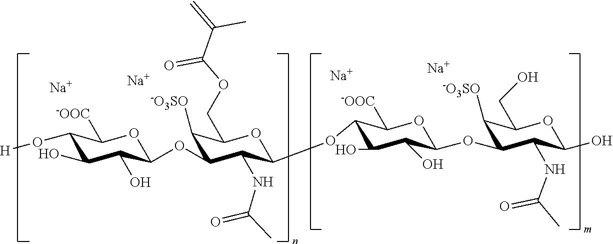 |
| | In some cases, n and m are independently integers selected from about 1 to about |
| | 500, wherein at least one of n and m is at least 1. In some cases, n and m are each |
| | at least 1. |
| 17 | 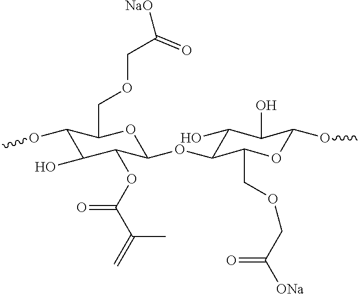 |
| | In some cases, structure 17 includes from about 1 to about 500 monosaccharide |
| | units. |
| 18 | 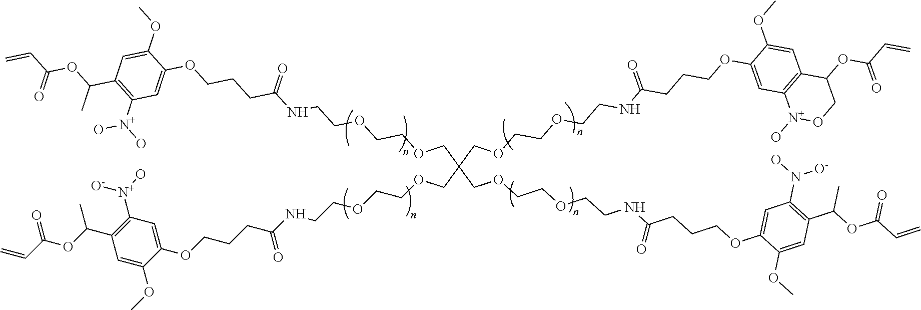 |
| | In some cases, each instance of n is independently an integer selected from about 1 |
| | to about 500. |
| 19 | 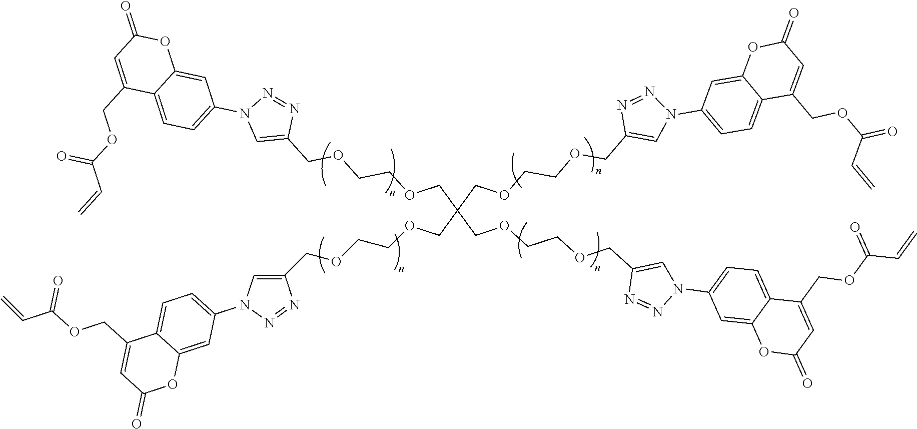 |
| | In some cases, each instance of n is independently an integer selected from about 1 |
| | to about 500. |
| 20 | 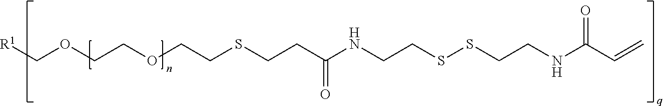 |
| | wherein R¹ is: i) a C₁ alkyl group, ii) a C₂-C₁₈ linear or branched saturated alkyl |
| | group, or iii) a C₃-C₈ cyclic saturated alkyl group substituted with 0-4 |
| | independently selected C₁-C₃ alkyl groups; wherein R¹ is substituted with q |
| | 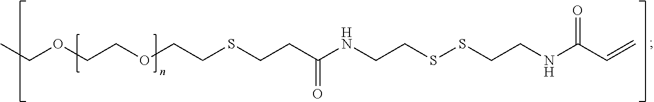 |
| | instances of |
| | the remaining substituents on R¹ are hydrogen, q is an integer from 2 to 32, and |
| | each instance of n is an integer selected from about 1 to about 500. |
| 21 | 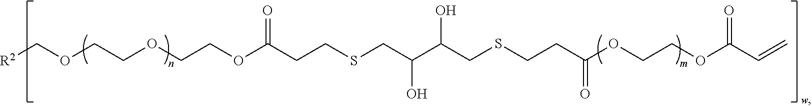 |
| | wherein R² is: i) a C₁ alkyl group, ii) a C₂-C₁₈ linear or branched saturated alkyl |
| | group, or iii) a C₃-C₈ cyclic saturated alkyl group substituted with 0-4 |
| | independently selected C₁-C₃ alkyl groups; |
| | wherein R² is substituted with w instances of |
| | 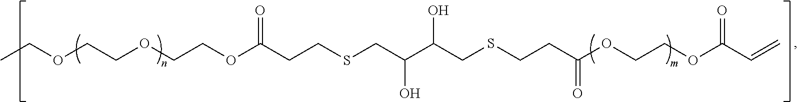 |
| | the remaining substituents on R² are hydrogen, w is an integer from 2 to 32, each |
| | instance of n is an integer independently selected from about 1 to about 500, and |
| | each instance of m is an integer independently selected from about 1 to about 500. |
| 22 | 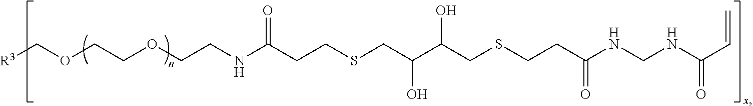 |
| | wherein R³ is: i) a C₁ alkyl group, ii) a C₂-C₁₈ linear or branched saturated alkyl |
| | group, or iii) a C₃-C₈ cyclic saturated alkyl group substituted with 0-4 |
| | independently selected C₁-C₃ alkyl groups; |
| | wherein R³ is substituted with x instances of |
| | 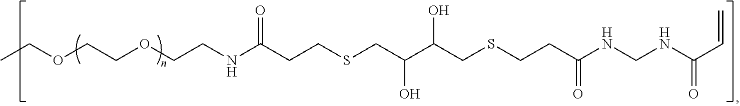 |
| | the |
| | remaining substituents on R³ are hydrogen, x is an integer from 2 to 32, and each |
| | instance of n is an integer independently selected from about 1 to about 500. |
| 23 | 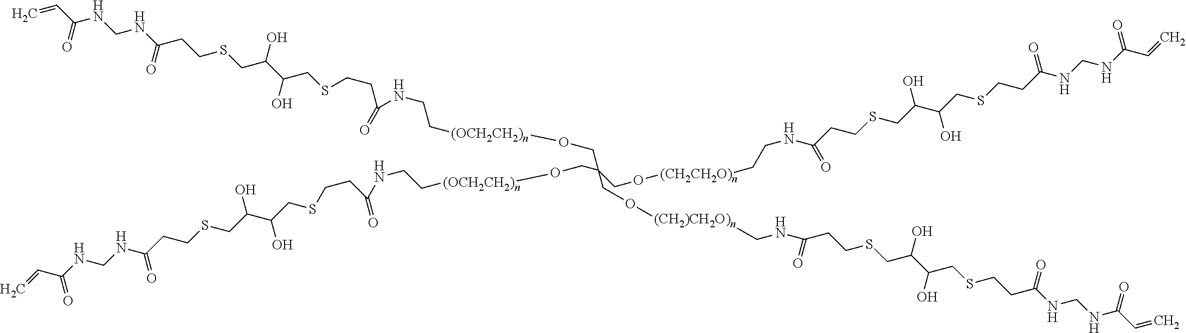 |
| | In some cases, each instance of n is independently an integer selected from about 1 |
| | to about 500. |
| 24 | 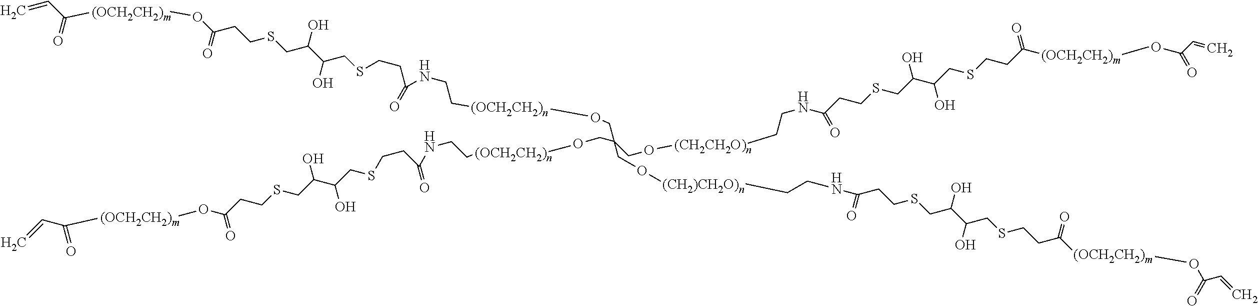 |
| | In some cases, each instance of n is independently an integer selected from about 1 |
| | to about 500. In some cases, each instance of m is independently an integer |
| | selected from about 1 to about 500. |
| 25 | 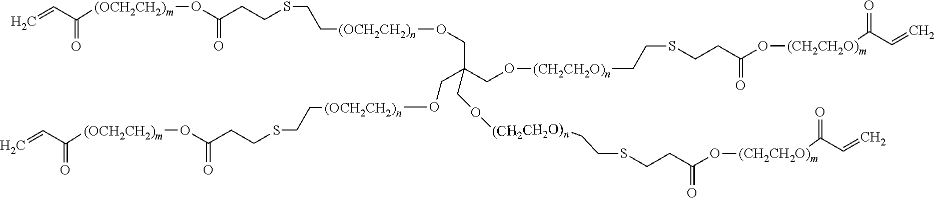 |
| | In some cases, each instance of n is independently an integer selected from about 1 |
| | to about 500. In some cases, each instance of m is independently an integer |
| | selected from about 1 to about 500. |
| 26 | 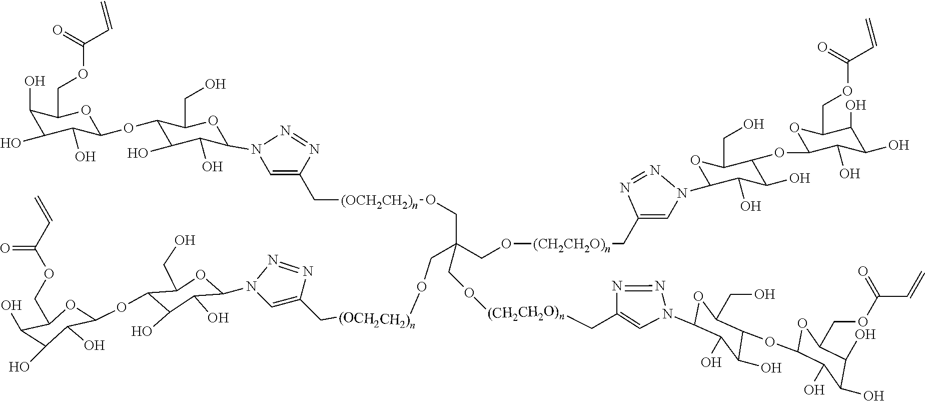 |
| | In some cases, each instance of n is independently an integer selected from about 1 |
| | to about 500. In some cases, each instance of m is independently an integer |
| | selected from about 1 to about 500. |

A polymer precursor can further comprise additional reagents that affect polymerization and polymer matrix properties. As examples, a polymer precursor can include a crosslinker, a porogen, a viscosity-modifying agent, an acid, a base, a catalyst, a salt, a photoinitiator, or a combination thereof. As another example, polymer precursors can include a crosslinker, a porogen, a photoinitiator, and a buffer.

As used herein, the term “crosslinker” denotes a species with two or more polymerizable groups. For example, where the polymerizable group is an ethylenically unsaturated group, a crosslinker would contain two or more ethylenically unsaturated groups. In another example, a crosslinker can contain three or more reactive centers for bifunctional polymer synthesis (e.g., the three methoxy groups of trimethoxybenzene in the context of polyester synthesis).

As used herein, the term “porogen” can denote a species that modulates the porosity of a polymer matrix. A porogen can be dispersed with the reactants before the polymerization process of forming the polymer matrix. Porogens typically diffuse out of polymer matrices following polymerization, leaving pores in the regions that they occupied. Porogen size, concentration, hydrophobicity, and hydrophilicity can thus influence pore density and pore size in polymer matrices. Examples of porogens consistent with the present disclosure include particles (e.g., polymeric, ceramic, metal, metal oxide, or hydrogel particles), polymers such as polyethylene glycol and alginate, and vesicles such as liposomes or micelles.

As used herein, the term “photoinitiator” can denote a species that generates a radical upon photoexcitation. In many cases, a photoinitiator included in a polymer precursor formulation is a type I photoinitiator, that is a molecule that generates radicals through intramolecular cleavage (e.g., homolysis) upon photoexcitation, or a type II photoinitiator, that is a molecule that abstract an electron or hydrogen atom from a co-initiator following photoexcitation. Examples of photoinitiators utilizable in the present methods include acetophenone, anisoin, anthraquinone, anthraquinone-2-sulfonic acid, benzil, benzoin, benzophenone, 3,3′,4,4′-benzophenonetetracarboxylic dianydride, 4-benzoylbiphenyl, 2-benzyl-2-(dimethylamino)-4′-morpholinobutyrophenone, dibenzosuberenone, 2,2-diethoxyacetophenone, 2-ethylanthraquinone, ferrocene, 2-isopropylthioxanthone, lithium phenyl (2,4,6-trimethylbenzoyl) phosphinate, methyl-2-benzoylbenzoate, and thiooxanthen-9-one.

### Nucleic Acid Capture Probes

In some cases, one or more surfaces of a fluidic device (e.g., first surface 101, second surface 102, or first and second surfaces 101 and 102 of channel 100 of FIG. 1) may contain one or more nucleic acid capture probes (e.g., the nucleic acid capture probes 1671 of FIG. 6E or the nucleic acid capture probes 1703 of FIG. 8A). One or more surfaces may contain, for example, about 1 to about 50,000,000 barcodes. The barcodes or subsets of the barcodes may be different from one another (e.g., each barcode may be unique). In some cases, a barcode (or a portion/subset of barcodes) may be associated with the location of the barcode on a surface (location coordinates (e.g., x-, y-coordinates) on a surface of a channel). In particular, a barcode may include a sequence that is unique (“spatially-addressed”) to a region or discrete site along a surface of the fluidic device. Each region or discrete site may contain a plurality of nucleic acid capture probes that share a common “spatially-addressed” sequence, while different discrete sites may be associated with different spatially-addressed sequences. Accordingly, the spot or region of origin of a barcode may be determined based on its spatially-addressed sequence. A discrete site or region that contains barcodes with common “spatially-addressed” sequences may cover an area, for example, of about 10 to about 10⁵ μm² along the first or second surface of the fluidic device. The first or second surface of the fluidic device may contain about 10 to about 10⁷ discrete sites or regions that contain barcodes with unique “spatially-addressed” sequences along one or more surfaces. In exemplary embodiments, a surface of a channel within a fluidic device may include about 5×103 to about 5×104 discrete spots. In further embodiments, a surface of a channel within a fluidic device may include about 5 to about 102 discrete spots per square millimeter. Similarly, the fluidic device may comprise one or more fluidic channels, each of which may include about 50 to 10⁷ spots or regions that contain barcodes with unique “spatially-addressed” sequences or spatially-addressed sequences that are associated with a region or discrete spot within each channel.

### Cellular and Biomolecular Analysis

“Cells” that may be cultured and assayed by methods and systems described herein may comprise any biological cells including, but not limited to, vertebrate, non-vertebrate, eukaryotic, mammalian, microbial, protozoan, prokaryotic, bacterial, archaea, insect, or fungal cells. In some embodiments, mammalian cells are assayed by methods and systems described herein. In particular, any population of mammalian cells which may be, or have been, induced, treated, modified, or genetically altered (i.e. genetically engineered) for use in a medical, industrial, environmental, or remedial process, may be analyzed by methods and systems described herein. In some embodiments, “cells” as used herein comprise genetically modified cells. In some embodiments, “cells” comprise stem cells that have been induced to differentiate. In some embodiments, “cells” refer to cells modified by CRISPR Cas9 techniques. In some embodiments, “cells” refer to cells of the immune system including, but not limited to, cytotoxic Tlymphocytes, regulatory T cells, CD4+ T cells, CD8+ T cells, natural killer cells, antigen-presenting cells, or dendritic cells. In some embodiments, “cells” refer to diseased cells such as cancer cells or virally infected cells.

A disclosed method can include one or more additional forms of cellular analysis that may be performed in parallel or in sequence with a nucleic acid copy number variation analysis method and/or a nucleic acid location analysis method. Such analyses may optionally be performed on a cell enclosed in a chamber, and may be performed in real-time or at defined timepoints. Accordingly, a single cell that is enclosed within a chamber within a fluidic device can be tracked independently of other cells that are present in the fluidic device. A nucleic acid copy number variation analysis method, a nucleic acid location analysis method, and/or one or more additional cellular analysis methods may be performed sequentially or in tandem. One or more cellular characteristics determined in a method can be selected from cytotoxicity, proliferative capacity, proliferation rate, activation status, cellular identity, purity, gene expression profile, transcriptome, surface marker expression, soluble factor secretion, activation status, epigenetic profile, sequence copy number (e.g., integrated viral copy number for transduced cells, plasmid copy number for transiently transfected cells, or gene copy number), or a combination thereof. Additional assays may include culture contamination assays including, but not limited to, viral, bacterial, yeast, mold, or mycoplasma assays, endotoxin assays, and cellular morphology assays.

An assay can include one or more assay components, which may be provided with or as part of a channel include, but are not limited to, capture elements such as nucleic acid capture probes (which may include capture sequences as disclosed elsewhere herein), primers for captured nucleic acid amplification, antibodies, and detectable labels. In some embodiments, such assay components may be attached to a surface of a fluidic device or a polymer matrix wall exclusively, or on combinations of such surfaces, either exclusively, or in combination with other reagents. In some embodiments, assay components may be provided after synthesizing gel chambers. However, assay components may be premixed with cells prior to introduction into a fluidic device, added to a fluidic device simultaneously with cells, added to a fluidic device after the addition of cells to the fluidic device, added to a fluidic device before the introduction of cells to the fluidic device, or a combination thereof. Assay components include, but are not limited to, lysing reagents, transcription reagents, reverse transcription reagents, antibodies, polymerases, primers, beads, and the like. In some embodiments, cellular or assay components may be attached or captured by capture elements on a polymer matrix wall.

(i) gRNA Analysis

A method disclosed herein can include detecting a guide ribonucleic acid (gRNA) associated with a genetic modification of the cell (i.e., gRNA may be the cellular nucleic acid released by the cell, captured on a nucleic acid capture probe, and sequenced). The cell can be transiently or stably transfected with a sequence encoding a gRNA specific for a particular genomic sequence. The guide RNA can be coupled to a barcode, an exogenous messenger RNA (e.g., a selection marker), a capture sequence (e.g., a poly A tail), or a combination thereof. The first cell or the aggregate of the first cells can express a Cas protein that can utilize the gRNA. Alternatively, a Cas protein can be delivered to the cells, for example in a chitosan particle or liposome. Cell growth, movement, or other characteristic or characteristics can then be correlated with a genomic edit imparted by a particular gRNA sequence. In one such method, the cells can be lysed to release guide RNA, the guide RNA can optionally be captured on a nucleic acid capture probe, and then be used as a template for generating a cDNA molecule comprising a complement of the guide RNA sequence, and optionally additional sequences coupled to the guide RNA such as the exogenous mRNA, the barcode, or a combination thereof. The cDNA molecule can be coupled to a spatial location tag corresponding to a unique location within the channel of the fluidic device.

### (ii) Proliferation Rate

In some aspects, determining a characteristic of the one or more cells includes determining a proliferation rate. It is understood that the term “proliferation rate” may include a measure of a lack of proliferation. Proliferation rate can be determined by counting cells at least partially enclosed by the one or more chambers generated during an assay. For example, the one or more cells can be counted periodically (e.g., with fluorescence or brightfield imaging) following at least partial enclosure within the one or more compartments (e.g., chambers) to determine a rate of change in the number of cells. Separate proliferation rates can be determined for each cell or collection of cells enclosed by a unique chamber or collection of chambers. In some embodiments, cells may be stained with a membrane or intracellular dye for determining proliferation by dye dilution so that an independent measure of cell proliferation may be obtained. Exemplary intracellular dyes for dye dilution include, but are not limited to, Hoechst 33342, carboxyfluorescein succinimidyl ester (CFSE), and the like. After counts are recorded for each chamber, further assays may be conducted on the clonal populations within the chambers to identify the cell types, for example, by an assessment of cell surface proteins, cell protein secretions, transcriptome, or the like.

(iii) Soluble Factor Analysis

In some aspects, determining a characteristic of the one or more cells includes detecting a soluble factor secreted by the one or more cells. Soluble factor analysis can include disposing a capture surface (e.g., a bead) comprising an affinity reagent (e.g., an aptamer or an antibody) that binds the soluble factor adjacent to one or more cells and detecting the soluble factor bound to the capture surface. Disposing the capture surface adjacent to the one or more cells can denote enclosing or at least partially enclosing the capture surface with the one or more cells within the one or more chambers, and optionally removing non-enclosed capture surfaces from the channel of the fluidic system. The capture surface can be loaded into the channel at a controlled density, for example 1 capture surface per about 100, 50, 10, 5, 1, 0.5, 0.1, 0.05, 0.01, 0.005, or 0.001 mm² of the channel.

In an exemplary embodiment, the capture surface comprises a bead. As used herein, the term “bead” can denote a microparticle or a nanoparticle, such as a ceramic, metal, metal oxide, polymer, or saccharide-based 30 to 10000 μm particle. However, further capture surfaces, including nanotubes, nucleic acid nanostructures, and antibody Fc domains. The capture surface affinity reagent can, as non-limiting examples, include antibodies, antibody fragments, aptamers, affimers, or a combination thereof.

Soluble factor detection may be performed with a bispecific affinity reagent capable of simultaneously binding to a cell and to a soluble factor secreted by the cell. The bispecific affinity reagent can be coupled to a target cell of interest and then used to capture soluble factors secreted by the cell. In this way, the bispecific affinity reagent may couple the soluble factor to the surface of the cell. The soluble factor may then be detected, for example by coupling a detectable affinity reagent such as a fluorescent antibody to the soluble factor coupled to the surface of the cell, and measuring the detectable affinity reagent. Unlike antibody modified beads that reside randomly within the chamber for capturing secretions that happen to diffuse to the beads, bispecific antibodies are coupled to the cell surface allowing for a more efficient capture of secretions emanated from the cell surface.

In a further exemplary embodiment, the soluble factor comprises a cytokine such as interferon-γ (IFN-γ) and interferon-α (IFN-α), an interleukins such as interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-13 (IL-13), interleukin-15 (IL-15), interleukin-21 (IL-21), or interleukin-23 (IL-23), a colony stimulating factor (CSFs) such as granulocyte-macrophage colony stimulating factor (GM-CSF), granulocyte colony stimulating factor (G-CSF), or a tumor necrosis factors (TNF) such as TNF-α or TNF-β. In another embodiment, the secreted factor comprises an effector molecule such as a granzyme.

A soluble factor bound to a capture surface (e.g., an affinity reagent of a capture surface) can be detected by contacting the soluble factor bound to said capture surface with a labeled antibody configured to bind to the soluble factor, and detecting the labeled antibody. Multiple soluble factors can be detected in a single assay by providing a capture surface or plurality of capture surfaces that comprise a plurality of affinity reagents configured to bind the plurality of soluble factors, contacting the plurality of soluble factors bound to the capture surface or plurality of capture surfaces with a plurality of labeled antibodies configured to bind to the plurality of soluble factors, and detecting a plurality of labels coupled to the plurality of antibodies.

### (iv) Transcriptomic Analysis

In some aspects, determining a characteristic of the one or more cells includes sequencing at least a portion of a transcriptome of the one or more cells. In some methods disclosed herein, sequence copy number (e.g., gene copy number) and/or location (e.g., genomic location) are performed in tandem with transcriptomic analysis. A fluidic device may contain nucleic acid capture probes that are configured to capture a nucleic acid from a cell and then be extended using the nucleic acid as a template. The fluidic device can include nucleic acid capture probes that are configured to couple to a single type of cellular nucleic acid or multiple types of nucleic acid capture probes that are configured to couple to multiple sequences and/or types of target nucleic acids. Multiple types of nucleic acid capture probes can be disposed within a common discrete spot along the surface of the fluidic device. In some cases, a fluidic device includes a single type of nucleic acid capture probe for transcriptomic analysis and sequence copy number and/or location analysis. In particular, a fluidic device can include nucleic acid capture probes with a single capture sequence that is configured to capture (1) mRNA and (2) DNA fragments, amplicons, circularization probes, extended random primers, or a combination thereof. Alternatively, a fluidic device can include a first population of nucleic acid capture probes that are configured to capture mRNA for transcriptomic analysis and a second population of nucleic acid capture probes that are configured to capture DNA fragments, amplicons, circularization probes, extended random primers, or a combination thereof for sequence copy number (e.g., gene copy number) and/or sequence location analysis. The first and second populations of nucleic acid capture probes may be colocalized within discrete spots along a surface of a fluidic device.

The nucleic acid capture probes may contain spatial barcode sequences that are uniquely associated with a discrete spot along a surface of the fluidic device. Each discrete spot may include multiple nucleic acid capture probes that each include a common spatial barcode sequence. In some cases, each discrete spot is enclosed or partially enclosed by at most one chamber. In such cases, extended nucleic acid capture probes (which contain the spatial barcodes) and/or mRNA extended using the nucleic acid capture probes as templates (which thus contain complements of the spatial barcodes) may be associated with a cell or cells enclosed by one or more chambers. The nucleic acid capture probes may also contain unique molecular identifiers to facilitate mRNA quantitation by normalizing sequencing counts of extended nucleic acid capture probes and/or mRNA extended using the nucleic acid capture probes as templates. For example, the number of instances of each mRNA sequence may be determined based on the number of unique molecular identifier sequences associated with that mRNA sequence.

In some cases, mRNA and/or nucleic acid capture probe extension involves reverse transcription. Reverse transcription reagents may comprise conventional reagents for reverse transcription; namely, a reverse transcriptase (such as, a Moloney murine leukemia virus (MMLV)), dNTPs, optional RNase inhibitor, buffer.

The sequencing step may be carried out at the sites of the captured mRNAs (in situ) or cDNAs (e.g., nucleic acid capture probe extensions generated using a reverse transcriptase) may include a spatial barcode and be eluted and sequenced on a separate sequencing instrument (“external” sequencing). For in situ sequencing, further steps may include (i) amplifying the complementary DNAs, e.g. by bridge amplification, or like method, (ii) sequencing the amplified complementary DNAs, e.g. by a sequencing-by-synthesis technique, and (iii) determining relative expression of the mRNAs for the cells of each of the chambers. For external sequencing, further steps may include (i) providing capture elements comprising spatial barcodes, (ii) synthesizing cDNAs comprising spatial barcodes, and (iii) eluting and sequencing the cDNAs and correlating each cDNA with a chamber location by its spatial barcode.

### (v) Cytotoxicity

In a further aspect, the one or more cells comprises an effector cell, and determining the characteristic of the one or more cells comprises measuring a cytotoxicity of the effector cell. Such a method may be performed by at least partially enclosing one or more target cells with the effector cell, and counting dead cells, viable cells, or a combination thereof from among the one or more target cells. As non-limiting examples, the target cells may comprise a sample of tumor cells of a patient, or target cells may be from a cell line, e.g. tumor cell line, such as, hepatic tumor cell line, SK-HEP-1, Chava et al, J. Vis. Exp., 2020 Feb. 22: (156): 10.3791/60714. Examples of effector cells include Tc1 cells, Tc2 cells, Tc9 cells, Tc17 cells, Tc22 cells, natural killer cells. In some embodiments, the effector cell is engineered for a therapeutic purpose. For example, the effector cell may express a chimeric antigen receptor that confers cytotoxicity against a particular cancer.

A cytotoxicity assay may include loading the effector cell and target cells into the channel (either simultaneously or sequentially). In some aspects, the target cells are loaded into channel where they optionally adhere to a surface prior to effector cell loading. In other aspects, the effector cell is loaded into the channel prior to target cell loading. The effector cell may be at least partially enclosed with the target cells inside one or more chambers. The target cells and effector cells are then incubated. The incubation may be performed in the presence of a vital dye that generates an optical signal in response to a characteristic of viable cells or dead cells to facilitate dead cell enumeration. The method may include counting the number of target cells killed per effector cell. The method may also include counting the number of target cells killed per unit time. Alternatively or in addition thereto, the target cells may be pre-treated with a dye that internalizes into the target cells prior to loading into the channel. In addition to target cell killing, effector cells can be identified based on other cellular characteristics disclosed herein, such as their secretome, transcriptome, or the like.

### (vi) Cellular Activation

In some aspects, the characteristic of the one or more cells comprises activation. Cellular activation can be detected using numerous assays disclosed herein, including surface marker expression, soluble factor secretion, transcriptomic analysis, proliferation or changes in proliferation, changes in morphology, change in cytotoxicity, or a combination thereof. As non-limiting examples, these methods are broadly amenable to detecting activation caused by contact between a second cell and a cell of the one or more cell, a soluble factor secreted by the second cell and the cell of the one or more cells, and combinations thereof. In a particular aspect of the present disclosure, determining activation comprises detecting a surface marker of the one or more cells. For example, the method can comprise contacting the one or more cells with a binding agent configured to bind to the surface marker and detecting the binding agent.

### Fluidic and Optical Systems

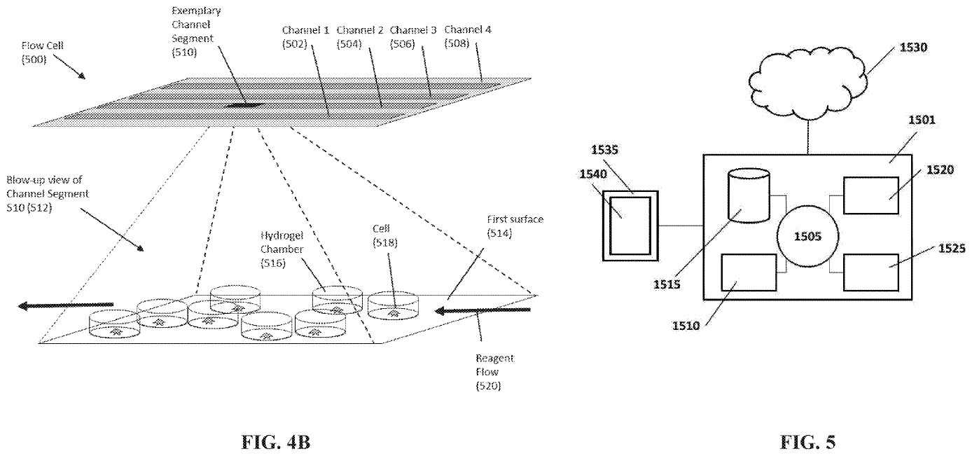

FIG. 4A is an example of a system for carrying out the disclosed methods. Flow cell (500) is a component of a fluidic device that provides channels for carrying out a variety of assays and liquid handling components under programmable control for delivering samples and reagents to the channels. In this illustration, four channels (502, 504, 506, and 508) are shown. However, as detailed elsewhere herein, systems of the present disclosure can utilize flow cells with fewer or greater numbers of channels.

The system of FIG. 4A includes an optical system (521) for photosynthesizing chambers at locations of cells or other analytes in the channels (502, 504, 506, and 508) of the flow cell (500) and for collecting images and other optical signals. The optical system (521) includes a light source (522) that generates a light beam (523) of appropriate wavelength light (e.g. UV light) for synthesizing chambers (e.g., hydrogel chambers) in the flow cell (500). The light beam (523) that passes through an appropriate photo-mask or beam-shaping or beam steering (Galvo) system (524) for shaping a beam to synthesize a desired structure or structures in a channel. For example, the beam shaping system (524) may project circular patterns of light from the light source (522) onto the flow cell (500) to cause a polymer precursor to photopolymerize in the shape of a cylindrical chamber (e.g., chamber 516 in FIG. 4B). In some embodiments, this beam shaping system (524) includes a digital micromirror device (DMD). In other embodiments, a physical photo-mask may be employed. Reflected light from DMD (524) is shaped using conventional optics, e.g. collimating optics (528), and is directed through objective lens system (534) into channel 2 segment (510) . . . . In other embodiments, the beam shaping system (524) can include a virtual mask (e.g., a computer code or a digital system). The virtual mask may render one or more electrodes, or arrays of electrodes, to produce spatially-modulated energy (e.g., light, electrical current, etc.) to form the patterned polymer matrix in the flow cell (500). The virtual mask may also block a portion of light from the light source (522). When a virtual mask is utilized in this manner, the virtual mask can prevent light from being emitted to a location where analyte (e.g., a cell 518) is present within the flow cell (500). In exemplary embodiments, the light is directed by one or more dichroic mirrors (530 and 531).

Chamber position, shape and polymer matrix wall thickness can be determined at least in part from cell position information determined from images collected by detector (532). Objective (534) and flow cell (500) move relative to one another in the xy-directions (536) to photosynthesize chambers at any position in any of the channels. In some embodiments, the flow cell (500) moves and optical system (521) is stationary. The system may utilize light from a light source (599), such as a homogenized light condenser, that is positioned on an opposite side of the flow cell as the optical system and directs light through the flow cell (500) to the objective (534). To achieve this functionality, the light source positioned on the opposite side of the flow cell (599) can be configured to move in tandem with the optical system (521), or the light source (599) and optical system can be stationary and the flow cell (500) can be moved to the region illuminated by the light source (599) and from which light is collected by the objective (534). In some embodiments, objective (534) may also direct light beam (527) from light source (529) to targets, such as cells, on first surface (514, see FIG. 4B) and collect optical signals, such as fluorescent signals, from assays taking place on first surface (514). Optical signal collection can also be carried out with a separate objective. Information collected by detector (532), particularly cellular positions in their respective channels, is employed by computer (538) and/or subsidiary controllers to direct DMD (524) and translation devices controlling the relative positions of objective (534) and flow cell (500) to synthesize hydrogel chambers of the appropriate shape and size at the appropriate locations.

FIG. 4B provides a blown-up view of the exemplary channel segment (510) of the flow cell of FIG. 4A. On first surface (514) of channel 2 (504) a plurality of cells, e.g. (518), are each enclosed by a hydrogel chamber, e.g. (516).

One of ordinary skill in the art would recognize that optical systems with different configurations than those of FIGS. 4A and 4B may be employed for carrying out these functions. In some embodiments, a plurality of DMD-objective subsystems for synthesizing hydrogel structures may be employed to increase the speed of synthesis by synthesizing multiple structures simultaneously.

### Computer Systems

The present disclosure provides computer systems that are programmed to implement methods of the disclosure. FIG. 5 shows a computer system 1501 that may be programmed or otherwise configured to perform methods described herein. The computer system 1501 can regulate various aspects of the present disclosure, such as, for example, identifying a biological component, detecting a barcode, controlling a spatial light modulator to shape a light beam, providing energy from an energy source, or detecting or measuring a local parameter using a sensor. The detector may be a camera (e.g., a fluorescent camera), such as a charged coupled device (CCD) camera capable of collecting optical signals and position information from a plurality of sources distributed over a planar region. The computer system 1501 can be an electronic device of a user or a computer system that may be remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that may be in communication with the Internet. The network 1530 in some cases may be a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit may be an application specific integrated circuit (ASIC).

The storage unit 1515 can store files, such as drivers, libraries, and saved programs. The storage unit 1515 can store user data, e.g., user preferences and user programs. The computer system 1501 in some cases can include one or more additional data storage units that are external to the computer system 1501, such as located on a remote server that may be in communication with the computer system 1501 through an intranet or the Internet.

The computer system 1501 can communicate with one or more remote computer systems through the network 1530. For instance, the computer system 1501 can communicate with a remote computer system of a user (e.g., a laptop, a personal computer, a tablet, or a mobile phone). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that may be carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, an image of a biological component, a barcode, a signal or measurement of a local parameter. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, identify a biological component, detect a barcode, generate a spatial modulating element (e.g., a mask), provide energy from an energy source, detect or measure a local parameter using a sensor, etc.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

What is claimed is:

1. A method for DNA analysis, the method comprising:
(a) hybridizing a first restriction enzyme to a first restriction enzyme binding site of a DNA molecule of a cell;
(b) hybridizing a second restriction enzyme to a second restriction enzyme binding site of the DNA molecule, wherein a first handle or a portion of the first handle and a target nucleic acid sequence are disposed between the first restriction enzyme binding site and the second restriction enzyme binding site of the DNA molecule;
(c) cleaving the DNA molecule with the first and second restriction enzymes, wherein the cleaving generates a nucleic acid fragment comprising:
i) the first handle or a portion of the first handle,
ii) the target nucleic acid sequence, and
iii) a flanking DNA sequence from the DNA molecule disposed at an end of the nucleic acid fragment;
(d) hybridizing the nucleic acid fragment or an amplicon generated from the nucleic acid fragment to a nucleic acid capture probe;
(e) extending the nucleic acid capture probe using at least a portion of the nucleic acid fragment or the amplicon as a template, thereby generating an extended nucleic acid capture probe comprising a complement of the target nucleic acid sequence and a complement of the flanking DNA sequence; and
(f) sequencing the extended nucleic acid capture probe or a nucleic acid generated from the extended nucleic acid capture probe.

2. The method of claim 1, further comprising inputting the cell into the fluidic device.

3. The method of claim 1, wherein the nucleic acid fragment further comprises an additional flanking DNA sequence from the DNA molecule, wherein the flanking DNA sequence and the additional flanking DNA sequence are disposed at opposite ends of the nucleic acid fragment.

4. The method of claim 1, wherein the first restriction enzyme binding site is disposed within the first handle.

5. The method of claim 1, wherein the first restriction enzyme binding site is disposed at a first end of the first handle, the target nucleic acid sequence is coupled to a second end of the first handle, and the first end of the first handle is opposite the second end of the first handle.

6. The method of claim 1, wherein the nucleic acid fragment further comprises a second handle or a portion of the second handle, and wherein the nucleic acid fragment comprises, in order:
i) the flanking DNA sequence, the first handle, the target nucleic acid sequence, and the portion of the second handle;
ii) the flanking DNA sequence, the first handle, the target nucleic acid sequence, and the second handle;
iii) the first handle, the target nucleic acid sequence, the second handle, and the flanking DNA sequence;
iv) the portion of the first handle, the target nucleic acid sequence, the second handle, and the flanking DNA sequence; or
v) the flanking DNA sequence, the first handle, the target nucleic acid sequence, the second handle, and an additional flanking DNA sequence from the DNA molecule.

7. The method of claim 6, wherein the second handle comprises a reverse complement of the first handle.

8. The method of claim 1, wherein the DNA molecule is inside of the cell during the cleaving, and the method further comprises releasing the nucleic acid fragment from the cell following the cleaving.

9. The method of claim 1, further comprising releasing the DNA molecule from the cell prior to the cleaving.

10. The method of claim 8, wherein the releasing comprises lysis.

11. The method of claim 1, further comprising inputting the cell into the fluidic device and synthesizing a chamber that co-encloses the cell with the nucleic acid capture probe.

12. The method of claim 1, wherein the DNA molecule comprises genomic DNA, plasmid DNA, vector DNA, extrachromosomal circular DNA (eccDNA), or a combination thereof.

13. The method of claim 1, wherein the target nucleic acid sequence comprises a knock-in gene.

14. The method of claim 1, wherein the cell comprises a plurality of instances of the target nucleic acid sequence.

15. The method of claim 1, wherein the first handle, the portion of the first handle, a second handle disposed adjacent to the target nucleic acid sequence and comprising the second restriction enzyme binding site, or a portion of the second handle of the nucleic acid fragment hybridizes to the nucleic acid capture probe.

16. The method of claim 1, wherein the extended nucleic acid capture probe comprises a complement of the first handle, the portion of the first handle, a second handle disposed adjacent to the target nucleic acid sequence and comprising the second restriction enzyme binding site, a portion of the second handle, or a combination thereof.

17. The method of claim 1, wherein the nucleic acid fragment further comprises a unique molecular identifier.

18. The method of claim 1, wherein the nucleic acid capture probe comprises a spatial barcode associated with a location of the nucleic acid capture probe within the fluidic device.

19. The method of claim 1, further comprising hybridizing the extended capture probe to a barcode primer, extending the extended capture probe using at least a portion of the barcode primer as a template, and extending the barcode primer using at least a portion of the extended capture probe as a template.

20. The method of claim 19, wherein the barcode primer comprises a spatial barcode, wherein the extended capture probe is extended using at least a portion of the spatial barcode as a template.
